SEQUENCE LISTING (90 sequences)

SEQ ID NO: 1 - DNA, 324 bp, Homo sapiens
  source 1..324: mol_type = genomic DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 2 - protein, 107 aa, Homo sapiens [sequence omitted]

SEQ ID NO: 3 - DNA, 1440 bp, synthetic construct
  source 1..1440: mol_type = other DNA
atgcacagacctagacgtcgtggaactcgtccacctccactggcactgctcgctgctctcctcctggctgcacgtggtgctgatgcacaggtacaattgaaacaaagtggcgccgaactggtaaaacctggagcaagtgttaagatcagctgtaaaacatcaggatatacatttaccgacggatatatgaattgggtggaacaaaaacctggacaggggctcgaatggattggaaggattgacccagatagcggtaacacaagatacaaccagaaatttcaaggcaaagcaactcttacacgcgataaatcatcatcaactgtctacatggacctgcgttcccttacttccgaggattcagctgtttattattgcgcacgggacggaacattttatggaacatacggttattggtatttcgacttctggggccaaggaactcaagtgacagtgtcatccgcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccgtgcccagcaccacctgtggcaggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 4 - protein, 479 aa, synthetic construct
MHRPRRRGTRPPPLALLAALLLAARGADAQVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - DNA, 27 bp, synthetic construct
  source 1..27: mol_type = other DNA
aaagctgtgtacaatttcgccacctgt

SEQ ID NO: 6 - protein, 9 aa, synthetic construct
KAVYNFATM

SEQ ID NO: 7 - DNA, 297 bp, Mus musculus
  source 1..297: mol_type = genomic DNA
atccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatg

SEQ ID NO: 8 - protein, 99 aa, Mus musculus [sequence omitted]

SEQ ID NO: 9 - DNA, 855 bp, Mus musculus
  source 1..855: mol_type = genomic DNA
ggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctgctacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtg

SEQ ID NO: 10 - protein, 285 aa, Mus musculus
GPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMV

SEQ ID NO: 11 - DNA, 2118 bp, synthetic construct
  source 1..2118: mol_type = other DNA
atgcacagaccaagaagaagaggaactagaccaccaccacttgcactccttgccgcccttttgcttgccgcccgtggtgctgatgcaaaagctgtgtacaatttcgccacctgtggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctgctacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgggtggtggcggatctggcggcggcggaagcggagggggaggctccggaggaggcggctccgaatccaaatacggtcccccctgccccccatgtcccgctccacctgtggctggtccctctgttttcctttttccccctaaacccaaagataccctcatgatttccagaacccccgaggtcacctgcgtcgtcgttgatgtaagccaagaagatcccgaagtccagttcaattggtatgtagacggtgttgaagtccataatgcaaaaacaaaacccagagaggaacagtttaattcaacctatcgtgtcgttagcgtactcaccgttcttcatcaagactggctcaatggaaaagaatataaatgtaaagttagcaacaaaggtctgcccagttcaatcgaaaaaacaattagcaaagccaaaggccaacctcgcgaaccccaagtctataccttgcccccttctcaggaagaaatgaccaaaaaccaagtttcactcacatgcctcgtaaaaggattctatccatcagacattgcagtagaatgggaatctaacggccaacctgaaaataattacaaaaccactcctcctgtcctcgattctgacggctcttttttcctttactccagattgactgttgataaatcccgctggcaggaaggtaacgttttttcttgttctgtgatgcacgaagccctccataacagattcactcaaaaatctctttctctctcccctggcaaataa

SEQ ID NO: 12 - protein, 705 aa, synthetic construct
MHRPRRRGTRPPPLALLAALLLAARGADAKAVYNFATCGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVGGGGSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 13 - DNA, 1452 bp, synthetic construct
  source 1..1452: mol_type = other DNA
atgcacagacctagacgtcgtggaactcgtccacctccactggcactgctcgctgctctcctcctggctgcacgtggtgctgatgcacaggtacaattgaaacaaagtggcgccgaactggtaaaacctggagcaagtgttaagatcagctgtaaaacatcaggatatacatttaccgacggatatatgaattgggtggaacaaaaacctggacaggggctcgaatggattggaaggattgacccagatagcggtaacacaagatacaaccagaaatttcaaggcaaagcaactcttacacgcgataaatcatcatcaactgtctacatggacctgcgttcccttacttccgaggattcagctgtttattattgcgcacgggacggaacattttatggaacatacggttattggtatttcgacttctggggccaaggaactcaagtgacagtgtcatccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 14 - protein, 483 aa, synthetic construct
MHRPRRRGTRPPPLALLAALLLAARGADAQVQLKQSGAELVKPGASVKISCKTSGYTFTDGYMNWVEQKPGQGLEWIGRIDPDSGNTRYNQKFQGKATLTRDKSSSTVYMDLRSLTSEDSAVYYCARDGTFYGTYGYWYFDFWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 2115 bp, synthetic construct
  source 1..2115: mol_type = other DNA
atgcacagacctagacgtcgtggaactcgtccacctccactggcactgctcgctgctctcctcctggctgcacgtggtgctgatgcaaaagctgtgtacaatttcgccacctgtggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctgctacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgggtggtggcggatctggcggcggcggaagcggagggggaggctccggaggaggcggctccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccgcttcacgcagaagtccctctccctgtctccgggtaaatag

SEQ ID NO: 16 - protein, 704 aa, synthetic construct
MHRPRRRGTRPPPLALLAALLLAARGADAKAVYNFATCGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 17 - DNA, 726 bp, synthetic construct
  source 1..726: mol_type = other DNA
caagttcagttgcaacagccaggcgccgaactcgttagacccggtacttcagtcaaactttcctgcaaagctagcggatataccttcacctcttattggatgcattggatcaaacagcgccctggacaaggcctggaatggattggcgtgatcgacccttcagacaactatactaaatacaaccagaaatttaaaggcaaggctacactcacagtagacacttcctcaagtaccgcatacatgcagttgagctctcttacctctgaagactctgcagtatatttttgcgctagggaaggatattacggatcatctccatggtttgcctattgggggcagggcactctcgtaaccgtttcctctggaggtggtggaagtggaggtggtggatccggtggaggtggatcacagatcgtaatgagtcagtctcctgccatactgtcagcatctcccggcgaaaaggtcaccatgacatgcagagcacgttcatcagtcagttacatacattggtatcagcaaaaacccggctcatcccccaaaccatggatctacgcaacatctaatctcgcatccggcgtaccaggcagattttctgggagcggttcaggcaccagttattccctcacaattacacgggttgaagcagaggatgcagcaacatattattgtcagcaatggtcttcaaaacccccaactttcggcggcggtacaaaacttgaaatatga

SEQ ID NO: 18 - protein, 226 aa, synthetic construct
QVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEI

SEQ ID NO: 19 - DNA, 2871 bp, synthetic construct
  source 1..2871: mol_type = other DNA
atgcacagaccaagaagaagaggaactagaccaccaccacttgcactccttgccgcccttttgcttgccgcccgtggtgctgatgcaaaagctgtgtacaatttcgccacctgtggatgcggaggtagtgggggaggtggctccgggggagggggatctatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgatgcggtatttcgagaccgccgtgtcccggcccggcctcgaggagccccggtacatctctgtcggctatgtggacaacaaggagttcgtgcgcttcgacagcgacgcggagaatccgagatatgagccgcgggcgccgtggatggagcaggaggggccggagtattgggagcgggaaacacagaaagccaagggccaagagcagtggttccgagtgagcctgaggaacctgctcggctgctacaaccagagcgcgggcggctctcacacactccagcagatgtctggctgtgacttggggtcggactggcgcctcctccgcgggtacctgcagttcgcctatgaaggccgcgattacatcgccctgaacgaagacctgaaaacgtggacggcggcggacatggcggcgcagatcacccgacgcaagtgggagcagagtggtgctgcagagcattacaaggcctacctggagggcgagtgcgtggagtggctccacagatacctgaagaacgggaacgcgacgctgctgcgcacagattccccaaaggcacatgtgacccatcaccccagatctaaaggtgaagtcaccctgaggtgctgggccctgggcttctaccctgctgacatcaccctgacctggcagttgaatggggaggagctgacccaggacatggagcttgtggagaccaggcctgcaggggatggaaccttccagaagtgggcatctgtggtggtgcctcttgggaaggagcagaattacacatgccgtgtgtaccatgaggggctgcctgagcccctcaccctgagatgggagcctcctccgtccactgactcttacatggtgggtggtggcggatctggcggcggcggaagcggagggggaggctccggaggaggcggctccgaatccaaatacggtcccccctgccccccatgtcccgctccacctgtggctggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggcggaggtggatctggagggggcggatctcaagttcagttgcaacagccaggcgccgaactcgttagacccggtacttcagtcaaactttcctgcaaagctagcggatataccttcacctcttattggatgcattggatcaaacagcgccctggacaaggcctggaatggattggcgtgatcgacccttcagacaactatactaaatacaaccagaaatttaaaggcaaggctacactcacagtagacacttcctcaagtaccgcatacatgcagttgagctctcttacctctgaagactctgcagtatatttttgcgctagggaaggatattacggatcatctccatggtttgcctattgggggcagggcactctcgtaaccgtttcctctggaggtggtggaagtggaggtggtggatccggtggaggtggatcacagatcgtaatgagtcagtctcctgccatactgtcagcatctcccggcgaaaaggtcaccatgacatgcagagcacgttcatcagtcagttacatacattggtatcagcaaaaacccggctcatcccccaaaccatggatctacgcaacatctaatctcgcatccggcgtaccaggcagattttctgggagcggttcaggcaccagttattccctcacaattacacgggttgaagcagaggatgcagcaacatattattgtcagcaatggtcttcaaaacccccaactttcggcggcggtacaaaacttgaaatatga

SEQ ID NO: 20 - protein, 956 aa, synthetic construct
MHRPRRRGTRPPPLALLAALLLAARGADAKAVYNFATCGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSMRYFETAVSRPGLEEPRYISVGYVDNKEFVRFDSDAENPRYEPRAPWMEQEGPEYWERETQKAKGQEQWFRVSLRNLLGCYNQSAGGSHTLQQMSGCDLGSDWRLLRGYLQFAYEGRDYIALNEDLKTWTAADMAAQITRRKWEQSGAAEHYKAYLEGECVEWLHRYLKNGNATLLRTDSPKAHVTHHPRSKGEVTLRCWALGFYPADITLTWQLNGEELTQDMELVETRPAGDGTFQKWASVVVPLGKEQNYTCRVYHEGLPEPLTLRWEPPPSTDSYMVGGGGSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSGGGGSGGGGSGGGGSQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEI

SEQ ID NO: 21 - DNA, 1560 bp, synthetic construct
  source 1..1560: mol_type = other DNA
atgcacagacctagacgtcgtggaactcgtccacctccactggcactgctcgctgctctcctcctggctgcacgtggtgctgatgcaagtataatcaactttgaaaaactgggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgctacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtga

SEQ ID NO: 22 - protein, 519 aa, synthetic construct
MHRPRRRGTRPPPLALLAALLLAARGADASIINFEKLGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 23 - DNA, 2868 bp, synthetic construct
  source 1..2868: mol_type = other DNA
atgcacagacctagacgtcgtggaactcgtccacctccactggcactgctcgctgctctcctcctggctgcacgtggtgctgatgcaagtataatcaactttgaaaaactgggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgctacaaccagagcaagggcggctctcataccatacaagtcattagcggatgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcgtctgtcgtggtgccgctcggtaaagagcagtactatacctgccacgtataccaccagggcttgccggaaccattgacattgaggtgggaacctccgccctcaacagtctctaatatggctggggggggaggcagtggaggcggtggttcaggtggtggtgggtccgggggaggaggatctgaatctaaatatggccccccttgtccaccgtgccctgctccacctgtggctggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgtggtgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggcggaggtggatctggagggggcggatctcaagttcagttgcaacagccaggcgccgaactcgttagacccggtacttcagtcaaactttcctgcaaagctagcggatataccttcacctcttattggatgcattggatcaaacagcgccctggacaaggcctggaatggattggcgtgatcgacccttcagacaactatactaaatacaaccagaaatttaaaggcaaggctacactcacagtagacacttcctcaagtaccgcatacatgcagttgagctctcttacctctgaagactctgcagtatatttttgcgctagggaaggatattacggatcatctccatggtttgcctattgggggcagggcactctcgtaaccgtttcctctggaggtggtggaagtggaggtggtggttccggtggaggtggatcacagatcgtaatgagtcagtctcctgccatactgtcagcatctcccggcgaaaaggtcaccatgacatgcagagcacgttcatcagtcagttacatacattggtatcagcaaaaacccggctcatcccccaaaccatggatctacgcaacatctaatctcgcatccggcgtaccaggcagattttctgggagcggttcaggcaccagttattccctcacaattacacgggttgaagcagaggatgcagcaacatattattgtcagcaatggtcttcaaaacccccaactttcggcggcggtacaaaacttgaaatatga

SEQ ID NO: 24 - protein, 955 aa, synthetic construct
MHRPRRRGTRPPPLALLAALLLAARGADASIINFEKLGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMAGGGGSGGGGSGGGGSGGGGSESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGKGGGGSGGGGSQVQLQQPGAELVRPGTSVKLSCKASGYTFTSYWMHWIKQRPGQGLEWIGVIDPSDNYTKYNQKFKGKATLTVDTSSSTAYMQLSSLTSEDSAVYFCAREGYYGSSPWFAYWGQGTLVTVSSGGGGSGGGGSGGGGSQIVMSQSPAILSASPGEKVTMTCRARSSVSYIHWYQQKPGSSPKPWIYATSNLASGVPGRFSGSGSGTSYSLTITRVEAEDAATYYCQQWSSKPPTFGGGTKLEI

SEQ ID NO: 25 - DNA, 1560 bp, synthetic construct
  source 1..1560: mol_type = other DNA
atgcacagacctagacgtcgtggaactcgtccacctccactggcactgctcgctgctctcctcctggctgcacgtggtgctgatgcaaagagcccttggtttaccactttgggatgcggaggtagtgggggaggtggctccgggggagggggatccatccagaaaacccctcaaattcaagtatactcacgccacccaccggagaatgggaagccgaacatactgaactgctacgtaacacagttccacccgcctcacattgaaatccaaatgctgaagaacgggaaaaaaattcctaaagtagagatgtcagatatgtccttcagcaaggactggtctttctatatcctggctcacactgagttcacccccactgagactgatacatacgcctgcagagttaagcatgccagtatggccgagcccaagaccgtctactgggatcgagacatgggtggcggcggtagcggcgggggcggaagcggcggaggaggctccggtgggggtggttcaggcccacactcgctgaggtatttcgtcaccgccgtgtcccggcccggcctcggggagccccggtacatggaagtcggctacgtggacgacacggagttcgtgcgcttcgacagcgacgcggagaatccgagatatgagccgcgggcgcggtggatggagcaggaggggcccgagtattgggagcgggagacacagaaagccaagggcaatgagcagagtttccgagtggacctgaggaccctgctcggctgctacaaccagagcaagggcggctctcacactattcaggtgatctctggctgtgaagtggggtccgacgggcgactcctccgcgggtaccagcagtacgcctacgacggctgcgattacatcgccctgaacgaagacctgaaaacgtggacggcggcggacatggcggcgctgatcaccaaacacaagtgggagcaggctggtgaagcagagagactcagggcctacctggagggcacgtgcgtggagtggctccgcagatacctgaagaacgggaacgcgacgctgctgcgcacagattccccaaaggcccatgtgacccatcacagcagacctgaagataaagtcaccctgaggtgctgggccctgggcttctaccctgctgacatcaccctgacctggcagttgaatggggaggagctgatccaggacatggagcttgtggagaccaggcctgcaggggatggaaccttccagaagtgggcatctgtggtggtgcctcttgggaaggagcagtattacacatgccatgtgtaccatcaggggctgcctgagcccctcaccctgagatgggagcctcctccatccactgtctccaacatggcgaccgttgctgttctggttgtccttggagctgcaatagtcactggagctgtggtggcttttgtgatgaagatgagaaggagaaacacaggtggaaaaggaggggactatgctctggctccaggctcccagacctctgatctgtctctcccagattgtaaagtgatggttcatgaccctcattctctagcgtga

SEQ ID NO: 26 - protein, 519 aa, synthetic construct
MHRPRRRGTRPPPLALLAALLLAARGADAKSPWFTTLGCGGSGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 27 - DNA, 750 bp, synthetic construct
  source 1..750: mol_type = other DNA
atgcacagacctagacgtcgtggaactcgtccacctccactggcactgctcgctgctctcctcctggctgcacgtggtgctgatgcagatatcgtgatgacacaatctccctattctctggcagtatccgcaggcgagaaagtcaccatgtcctgtcgctcctcccagtcactttattattccggaattaagaaaaatctgctcgcttggtaccaacagaaacctggccaatcacctaaacttcttatctactttacttctactcgacttccaggagtgcctgacagatttacagggtccggttctggaacagactacacactcactattacctccgtgcaggccgaagacatgggccattacttctgtcagcaaggcatttccacccctttgacattcggagatggaacaaagctggaaattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 28 - protein, 249 aa, synthetic construct
MHRPRRRGTRPPPLALLAALLLAARGADADIVMTQSPYSLAVSAGEKVTMSCRSSQSLYYSGIKKNLLAWYQQKPGQSPKLLIYFTSTRLPGVPDRFTGSGSGTDYTLTITSVQAEDMGHYFCQQGISTPLTFGDGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 6 aa, synthetic construct
HHHHHH

SEQ ID NO: 30 - protein, 4 aa, synthetic construct
GGGG

SEQ ID NO: 31 - protein, 5 aa, synthetic construct
GGGGG

SEQ ID NO: 32 - protein, 6 aa, synthetic construct
GGGGGG

SEQ ID NO: 33 - protein, 7 aa, synthetic construct
GGGGGGG

SEQ ID NO: 34 - protein, 8 aa, synthetic construct
GGGGGGGG

SEQ ID NO: 35 - protein, 9 aa, synthetic construct
GGGGGGGGG

SEQ ID NO: 36 - protein, 4 aa, synthetic construct
GGGS

SEQ ID NO: 37 - protein, 4 aa, synthetic construct
SGGG

SEQ ID NO: 38 - protein, 5 aa, synthetic construct
GGGGS

SEQ ID NO: 39 - protein, 5 aa, synthetic construct
SGGGG

SEQ ID NO: 40 - protein, 6 aa, synthetic construct
GGGGGS

SEQ ID NO: 41 - protein, 6 aa, synthetic construct
SGGGGG

SEQ ID NO: 42 - protein, 7 aa, synthetic construct
GGGGGGS

SEQ ID NO: 43 - protein, 7 aa, synthetic construct
SGGGGGG

SEQ ID NO: 44 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 45 - protein, 50 aa, synthetic construct
  VARIANT 1..50: note = This sequence may encompass 1-10 SSSSG repeating units
SSSSGSSSSGSSSSGSSSSGSSSSGSSSSGSSSSGSSSSGSSSSGSSSSG

SEQ ID NO: 46 - protein, 4 aa, synthetic construct
GGSG

SEQ ID NO: 47 - protein, 5 aa, synthetic construct
GGSGG

SEQ ID NO: 48 - protein, 5 aa, synthetic construct
GSGSG

SEQ ID NO: 49 - protein, 5 aa, synthetic construct
GSGGG

SEQ ID NO: 50 - protein, 5 aa, synthetic construct
GGGSG

SEQ ID NO: 51 - protein, 5 aa, synthetic construct
GSSSG

SEQ ID NO: 52 - protein, 15 aa, synthetic construct
GCGASGGGGSGGGGS

SEQ ID NO: 53 - protein, 10 aa, synthetic construct
GGGGSGGGGS

SEQ ID NO: 54 - protein, 15 aa, synthetic construct
GGGASGGGGSGGGGS

SEQ ID NO: 55 - protein, 15 aa, synthetic construct
GGGGSGGGGSGGGGS

SEQ ID NO: 56 - protein, 10 aa, synthetic construct
GGGASGGGGS

SEQ ID NO: 57 - protein, 20 aa, synthetic construct
GGGGSGGGGSGGGGSGGGGS

SEQ ID NO: 58 - protein, 5 aa, synthetic construct
GSGGS

SEQ ID NO: 59 - DNA, 12 bp, synthetic construct
  source 1..12: mol_type = other DNA
gggggtggttcc

SEQ ID NO: 60 - DNA, 12 bp, synthetic construct
  source 1..12: mol_type = other DNA
ggtgggtctggg

SEQ ID NO: 61 - DNA, 15 bp, synthetic construct
  source 1..15: mol_type = other DNA
gggtccgggggctcc

SEQ ID NO: 62 - DNA, 15 bp, synthetic construct
  source 1..15: mol_type = other DNA
ggtgggagcggtggt

SEQ ID NO: 63 - DNA, 15 bp, synthetic construct
  source 1..15: mol_type = other DNA
ggcagcggaagcgga

SEQ ID NO: 64 - DNA, 15 bp, synthetic construct
  source 1..15: mol_type = other DNA
gggagtgggggaggt

SEQ ID NO: 65 - DNA, 15 bp, synthetic construct
  source 1..15: mol_type = other DNA
ggtgggggaagtgga

SEQ ID NO: 66 - DNA, 15 bp, synthetic construct
  source 1..15: mol_type = other DNA
ggcagctcatctggt

SEQ ID NO: 67 - DNA, 45 bp, synthetic construct
  source 1..45: mol_type = other DNA
ggatgtggtgcatctggagggggaggctctggggggggtggatct

SEQ ID NO: 68 - DNA, 45 bp, synthetic construct
  source 1..45: mol_type = other DNA
gggtgtggtgctagtgggggtggcggatcaggtggaggcgggagc

SEQ ID NO: 69 - DNA, 30 bp, synthetic construct
  source 1..30: mol_type = other DNA
gggggcggaggatctgggggagggggatca

SEQ ID NO: 70 - DNA, 42 bp, synthetic construct
  source 1..42: mol_type = other DNA
ggggggggcgcttcaggcggaggtggaagtggtggaggaggt

SEQ ID NO: 71 - DNA, 45 bp, synthetic construct
  source 1..45: mol_type = other DNA
ggagggggaggttctggcggcgggggatcaggaggcggtgggagc

SEQ ID NO: 72 - DNA, 30 bp, synthetic construct
  source 1..30: mol_type = other DNA
ggtgggggggcgtcaggtggaggcggaagt

SEQ ID NO: 73 - DNA, 45 bp, synthetic construct
  source 1..45: mol_type = other DNA
ggcggcggaggttctggtgggggtggcagtggaggaggaggcagc

SEQ ID NO: 74 - DNA, 60 bp, synthetic construct
  source 1..60: mol_type = other DNA
ggaggtggaggtagtggcggtggtgggtcagggggaggcgggtccggtggcggtgggagt

SEQ ID NO: 75 - DNA, 12 bp, synthetic construct
  source 1..12: mol_type = other DNA
ggtggcggtagt

SEQ ID NO: 76 - DNA, 12 bp, synthetic construct
  source 1..12: mol_type = other DNA
gggggatctggt

SEQ ID NO: 77 - DNA, 15 bp, synthetic construct
  source 1..15: mol_type = other DNA
ggcagtggcggtagc

SEQ ID NO: 78 - DNA, 15 bp, synthetic construct
  source 1..15: mol_type = other DNA
ggagggagtggaggg

SEQ ID NO: 79 - DNA, 15 bp, synthetic construct
  source 1..15: mol_type = other DNA
gggtctggctcaggc

SEQ ID NO: 80 - DNA, 15 bp, synthetic construct
  source 1..15: mol_type = other DNA
ggttctggcggaggt

SEQ ID NO: 81 - DNA, 15 bp, synthetic construct
  source 1..15: mol_type = other DNA
ggtggtgggagtgga

SEQ ID NO: 82 - DNA, 15 bp, synthetic construct
  source 1..15: mol_type = other DNA
ggctcaagcagtgga

SEQ ID NO: 83 - DNA, 45 bp, synthetic construct
  source 1..45: mol_type = other DNA
ggctgtggggctagtgggggaggtggtagtggtggtggcggttcc

SEQ ID NO: 84 - DNA, 45 bp, synthetic construct
  source 1..45: mol_type = other DNA
ggatgtggggcctcaggtgggggtggcagcggtggtggagggtca

SEQ ID NO: 85 - DNA, 30 bp, synthetic construct
  source 1..30: mol_type = other DNA
ggtggcgggggctctggtggaggaggatct

SEQ ID NO: 86 - DNA, 42 bp, synthetic construct
  source 1..42: mol_type = other DNA
ggaggcggcgcttctgggggcgggggtagtgggggtggaggt

SEQ ID NO: 87 - DNA, 45 bp, synthetic construct
  source 1..45: mol_type = other DNA
ggtggaggtggaagtggaggagggggatcaggcggaggcgggagc

SEQ ID NO: 88 - DNA, 30 bp, synthetic construct
  source 1..30: mol_type = other DNA
ggagggggagcctctggcggtggaggatca

SEQ ID NO: 89 - DNA, 45 bp, synthetic construct
  source 1..45: mol_type = other DNA
gggggaggaggcagtggaggtgggggaagtggtggaggggggtct

SEQ ID NO: 90 - DNA, 60 bp, synthetic construct
  source 1..60: mol_type = other DNA
gggggtggaggatcaggaggcggtggttctgggggaggtggatccggcgggggtggtagt

(19) United States
(12) Patent Application Publication
(10) Pub. No.: US 2026/0248911 A1
(43) Pub. Date: Aug. 27, 2026

(54) MULTISPECIFIC MOLECULES FOR MODULATING T-CELL ACTIVITY, AND USES THEREOF

(71) Applicant: Regeneron Pharmaceuticals, Inc., Tarrytown, NY (US)

(72) Inventors: Christos KYRATSOUS, Irvington, NY (US); Kurt EDELMANN, Yorktown Heights, NY (US)

(73) Assignee: Regeneron Pharmaceuticals, Inc., Tarrytown, NY (US)

(21) Appl. No.: 18/872,463

(22) PCT Filed: Jun. 7, 2023
(86) PCT No.: PCT/US2023/068030

§ 371 Date: Dec. 6, 2024

Related U.S. Application Data
(60) Provisional application No. 63/349,770, filed on Jun. 7, 2022.

(51) Int. Cl. A61K 39/385; A61K 38/00; C07K 14/705; C07K 16/28; C12N 15/85

(52) U.S. Cl. CPC A61K 39/385; C07K 14/70539; C07K 16/2821; C07K 16/2887; C07K 16/2896; C12N 15/85; A61K 38/00; C07K 2317/622; C07K 2319/30

(57) ABSTRACT

The invention provides multispecific molecules that are capable of binding to an antigen-specific T cell receptor (TCR) expressed on a surface of a cell, said multispecific molecules comprising a first molecule and a second molecule, wherein the first molecule is a polypeptide comprising (i) a peptide (p) presented in the context of a major histocompatibility complex (MHC) molecule (pMHC complex), and (ii) a first multimerization domain, and the second molecule is a polypeptide comprising (i) a domain that specifically binds a molecule expressed on the surface of the cell expressing the TCR, and (ii) a second multimerization domain, and the use of such molecules for modulating T cell activity and treating diseases such as cancers, infections, and autoimmune disorders.

### CROSS-REFERENCE TO RELATED APPLICATIONS

This application claims the benefit of Provisional U.S. Application No. 63/349,770, filed Jun. 7, 2022, the contents of which is herein incorporated by reference in its entirety for all purposes.

### SEQUENCE LISTING

The instant application contains a Sequence Listing which has been submitted electronically in XML format and is hereby incorporated by reference in its entirety. Said XML copy, created on Jun. 1, 2023, is named 250298_000494_SL.xml and is 105,334 bytes in size.

### FIELD OF THE INVENTION

Described herein are multispecific molecules, and the use of such molecules for modulating T cell activity and treating diseases.

### BACKGROUND

One of the central components of the adaptive immune system is the T cell, which is activated to proliferate and differentiate into effector cells (CD8+ cytotoxic T cells or CD4+ helper T cells) in response to the recognition of specific antigens. Key to the molecular events that lead to T cell activation is engagement of the T cell receptor (TCR) with an antigenic peptide presented in the groove of a major histocompatibility complex (MHC) protein on an antigen-presenting cell (e.g., a dendritic cell). The TCR is a membrane-bound heterodimer with an antibody-like binding site that recognizes a specific antigen. In addition to antigen recognition by the TCR, activation of T cells requires a co-stimulatory signal produced by engagement of cell surface proteins on the T cell and the antigen-presenting cell. CD28 is a widely-recognized cell surface molecule expressed on T cells that binds to CD80/CD86 on antigen-presenting cells to provide the costimulatory signal. Class I MHC proteins are engaged by CD8+ T cells that can be activated to form cytotoxic T cells, while class II MHC proteins are engaged by CD4+ T cells that can be activated to produce helper T cells.

Not only can T cell be activated to proliferate and differentiate, T cells can be inhibited from proliferating, putting them into a state of tolerance (anergy) via a inhibitory signal produced by engagement of cell surface proteins on the T cell and the antigen-presenting cell. CTLA-4 is a widely-recognized cell surface molecule expressed on T cells that also binds to CD80/CD86 (with an affinity that is significantly higher than CD28 in vitro) on antigen-presenting cells to provide the inhibitory signal. Inhibition of the T cells induces T cell tolerance via cell cycle arrest.

In spite of the prior attempts to create molecules for modulating a T cell-induced immune response, there remains a need in the art for molecules that can effectively modulate the activity of T cells to either suppress (in the case of autoimmune disorders) or induce (in the case of infections or cancers) an immune response.

### SUMMARY OF THE INVENTION

In general, the present invention provides multispecific molecules (e.g., bispecific molecules) and uses thereof, in which the multispecific molecules comprise a first molecule for engaging a T cell receptor and a second molecule for engaging a T cell surface molecule to modulate an activity of the T cell. Modulation of the T cell activity includes activation and/or proliferation (e.g., where the immunomodulatory molecule is a co-stimulatory molecule, such as CD28) or suppression of activity, anergy, and/or T cell death (e.g., wherein the immunomodulatory molecule is a inhibitory molecule, such as CTLA-4). In various embodiments, the first molecule of the multispecific molecules comprises a fusion of a peptide and major histocompatibility complex (MHC) protein such that the peptide is positioned in the groove (peptide in the groove, or PiG) of the MHC protein for presentation to and engagement by the T cell receptor (TCR) of a T cell with specificity for the peptide, and the second molecule of the multispecific molecules comprises a domain for binding to a T cell surface molecule on the T cell with specificity for the peptide. In this manner, T cells with specificity for the peptide may be activated to generate an immune response (e.g., in the case of an infection or cancer), or inhibited to suppress an immune response (e.g., in the case of an autoimmune disorder).

In one aspect, the present invention provides a multispecific molecule that is capable of binding to an antigen-specific T cell receptor (TCR) expressed on a surface of a cell, said multispecific molecule comprising a first molecule and a second molecule, wherein: the first molecule is a polypeptide comprising (i) a peptide (p) presented in the context of a major histocompatibility complex (MHC) molecule (pMHC complex) that is capable of binding to an antigen-specific T cell receptor (TCR) expressed on a surface of a cell, and (ii) a first multimerization domain, and the second molecule is a polypeptide comprising (i) a domain that specifically binds a molecule expressed on the surface of the cell expressing the TCR, and (ii) a second multimerization domain.

In some embodiments of the multispecific molecule, the domain that specifically binds a molecule expressed on the surface of the cell expressing the TCR is an antigen-binding domain. In some embodiments, the antigen-binding domain is a monovalent antigen-binding domain. In certain embodiments, the antigen-binding domain binds to an immunomodulatory molecule (e.g., CD28 or CTLA4) on the surface of the cell expressing the TCR. In some embodiments, the domain that specifically binds a molecule expressed on the surface of the cell expressing the TCR is a small molecule, protein, fusion protein, peptide, aptamer, avimer or derivatives or fragments thereof. In certain embodiments, the domain that specifically binds a molecule expressed on the surface of the cell expressing the TCR is a T-cell co-immunomodulatory molecule, that upon binding the molecule on the surface of the cell expressing the TCR modulates the activity of the T cell (e.g., the domain can be a co-stimulatory molecule such as CD80 or CD40 or a inhibitory molecule such as PD-L1 or B7-H3). In certain embodiments, the domain that specifically binds a molecule expressed on the surface of the cell expressing the TCR is a protein or derivative or fragment thereof, wherein the protein is an adhesion molecule (e.g., ICAM).

In some embodiments of the multispecific molecule, the first and/or the second multimerization domain is an immunoglobulin Fc domain. In some embodiments, the first and/or the second Fc domain is a human IgG Fc domain, e.g., a human IgG1 Fc domain or a human IgG4 Fc domain, or a human IgM Fc domain. In some embodiments, the first and second Fc domains are the same. In some embodiments, the first Fc domain and/or the second Fc domain comprise a CH3 mutation to facilitate purification of the multispecific molecule. In some embodiments, the CH3 mutation is a knob-into-hole mutation or charged mutation. In some embodiments, the first Fc domain or the second Fc domain comprises a CH3 mutation to facilitate purification of the multispecific molecule via affinity chromatography. In some embodiments, the first and/or the second Fc domain exhibits enhanced Fcγ-receptor binding activity relative to wild-type human IgG1.

In some embodiments of the multispecific molecule, the antigen-binding domain of the second molecule comprises a Fab or single-chain Fv (scFv).

In some embodiments of the multispecific molecule, the first and/or the second multimerization domain comprises a second antigen-binding domain that specifically binds a cell-surface molecule. In some embodiments, such binding is used for immobilizing the multispecific molecule on a cell surface (which can be used as a multispecific carrier). In some embodiments, the second antigen-binding domain specifically binds a B-cell surface molecule or a tumor-associated antigen. In some embodiments, the B-cell surface molecule is CD5, CD19, CD20, CD21, CD22, CD23, CD24, CD25, CD27, CD30, CD34, CD35, CD38, CD180, or CD40. In one embodiment, the B-cell surface molecule is CD20. In one embodiment, the B-cell surface molecule is CD180. In some embodiments, the second antigen-binding domain comprises a scFv. In some embodiments, the scFv is attached to the C-terminus of the first and/or the second multimerization domain. An scFv linked to the C-terminus of a multimerization domain (e.g., an IgG Fc domain) is alternatively referred to herein as “a Stahl body”.

In some embodiments of the multispecific molecule, the pMHC complex displays a peptide in a class I MHC polypeptide or a fragment (e.g., the peptide binding groove), mutant or derivative thereof. In some embodiments, the pMHC complex comprises (i) a peptide; (ii) a β₂-microglobulin polypeptide or a fragment, mutant or derivative thereof, (iii) a class I MHC a chain domain or fragment, mutant or derivative thereof, and (iii) an immunoglobulin (Ig) Fc domain. In some embodiments, the pMHC complex comprises, from N-terminus to C-terminus, (i) a peptide, (ii) a β2-microglobulin polypeptide or a fragment, mutant or derivative thereof, (iii) a class I a chain domain or fragment, mutant, or derivative thereof, and (iv) an Ig Fc domain. In some embodiments, the pMHC complex comprises, from N-terminus to C-terminus, (i) a peptide, (ii) an optional first linker, (iii) a β₂-microglobulin polypeptide or a fragment, mutant or derivative thereof, (iv) an optional second linker, (v) class I MHC α chain domains 1, 2 and/or 3 or fragments, mutants or derivatives thereof, (vi) an optional third linker, and (vii) an Ig Fc domain. In some embodiments, the MHC comprises α1 and α2 domains of a class I MHC polypeptide, or fragments, mutants or derivatives thereof. In some embodiments, the MHC comprises α1, α2, and/or α3 domains of a class I MHC polypeptide, or fragments, mutants or derivatives thereof. In some embodiments, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H 2L, H-2Q, H-2M, and H-2T.

In some embodiments of the multispecific molecule, the pMHC complex displays a peptide in a class II MHC polypeptide or a fragment (e.g., the peptide binding groove), mutant or derivative thereof. In some embodiments, the pMHC complex comprises (i) a peptide, (ii) a class II MHC α chain domain or a fragment, mutant or derivative thereof, (iii) a class II MHC β chain domain or a fragment, mutant or derivative thereof, and (iv) an Ig Fc domain. In some embodiments, the MHC class II α chain consists of the extracellular domains of the class II α chain. In some embodiments, the MHC class II beta chain consists of the extracellular domains of the class II beta chain. In some embodiments, the pMHC complex comprises, from N-terminus to C-terminus, (i) a peptide, (ii) class II MHC α chain extracellular domains or fragments, mutants or derivatives thereof, (iii) class II MHC β chain extracellular domains or fragments, mutants or derivatives thereof, and (iv) an Ig Fc domain. In some embodiments, the pMHC complex comprises, from N-terminus to C-terminus, (i) a peptide, (ii) class II MHC β chain extracellular domains or fragments, mutants or derivatives thereof, (iii) class II MHC α chain extracellular domains or fragments, mutants or derivatives thereof, and (iv) an Ig Fc domain. In some embodiments, the pMHC complex comprises, from N-terminus to C-terminus, (i) a peptide, (ii) an optional first linker, (iii) class II MHC α chain domains 1 and 2 or fragments, mutants or derivatives thereof, (iv) an optional second linker, (v) class II MHC β chain domains 1 and 2 or fragments, mutants or derivatives thereof, (vi) an optional third linker, and (vii) an Ig Fc domain. In some embodiments, the pMHC complex comprises, from N-terminus to C-terminus, (i) a peptide, (ii) an optional first linker, (iii) class II MHC β chain domains 1 and 2 or fragments, mutants or derivatives thereof, (iv) an optional second linker, (v) class II MHC α chain domains 1 and 2 or fragments, mutants or derivatives thereof, (vi) an optional third linker, and (vii) an Ig Fc domain. In certain embodiments, the MHC comprises α and β polypeptides (or fragments thereof, e.g., the α1 and β1 domains) of a human class II MHC complex selected from the group consisting of HLA DP, HLA-DR, HLA-DQ, HLA-DM and HLA-DO. In another specific embodiment, the MHC comprises α and β polypeptides (or fragments thereof, e.g., the α1 and β1 domains) of a murine H-2A or H-2E class II MHC complex.

In some embodiments of the multispecific molecule, the T-cell surface molecule is a T-cell immunomodulatory molecule to which the domain that specifically binds a molecule expressed on the surface of the cell expressing the TCR specifically binds is a T-cell co-stimulatory molecule. In some embodiments, the T-cell co-stimulatory molecule is selected from the group consisting of CD28, CD40L, ICOS, CD27, OX40, 4-1BB, GITR, HVEM, galectin 9, LFA-1, DR3, CD30, SLAM, 2B4, CD226, TIM1, TIM2, and CD2.

In some embodiments of the multispecific molecule, the T-cell surface molecule is a T-cell immunomodulatory molecule to which the domain that specifically binds a molecule expressed on the surface of the cell expressing the TCR specifically binds is a T-cell inhibitory molecule. In some embodiments, the inhibitory molecule is selected from the group consisting of BTLA, B7-1, B7-H1, CD160, CTLA4, LAG3, LAIR1, MHC-I, PD1, TIGIT, or TIM3.

In some embodiments of the multispecific molecule, the peptide consists of about 5 to about 40 amino acid residues, from about 6 to about 30 amino acid residues, from about 8 to about 20 amino acid residues, or about 9, 10, or 11 amino acid residues.

In some embodiments of the multispecific molecule, the peptide is derived from a viral antigen. In some embodiments, the viral antigen is derived from a virus selected from the group consisting of adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, and zika.

In some embodiments of the multispecific molecule, the peptide is derived from a bacterial antigen. In some embodiments, the bacterial antigen is an antigen associated with a bacterium that is resistant to conventional antibiotic treatments, such as methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, or Clindamycin-resistant group B Streptococcus.

In some embodiments of the multispecific molecule, the peptide is derived from a tumor-associated antigen. In some embodiments, the tumor-associated antigen is selected from the group consisting of adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRvIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A 2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1, -A2, -A3, -A4, -A6, -A9, -A10, -A12, -C1, and -C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPPIR3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, WT-1. In some embodiments, the peptide is a neo-antigen. In some embodiments, the peptide is a tumor specific antigen.

In some embodiments of the multispecific molecule, the peptide is derived from an antigen associated with an autoimmune disorder. In some embodiments, the antigen is selected from the group consisting of gliadin (celiac disease; e.g., (i) α-gliadin fragment corresponding to amino acids 57-73 or (ii) γ-gliadin fragment corresponding to amino acids 139 153 or (iii) ω-gliadin fragment corresponding to amino acids 102-118), GAD 65, IA-2 and insulin β chain (for type 1-diabetes), glatiramer acetate (GA) (for multiple sclerosis), acetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)). In some embodiments, the antigen associated with an autoimmune disorder is selected from the group consisting of IL-4R, IL-6R, and DLL4.

In some embodiments of the multispecific molecule, the first immunoglobulin Fc domain, the second immunoglobulin Fc domain, or both the first and second immunoglobulin Fc domains is capable of binding to another Fc domain or an Fcγ-receptor expressed on a T-cell, and clustering four or more T-cells bound to the multispecific molecule.

In another aspect, the present invention provides a pharmaceutical composition comprising a multispecific molecule as discussed above or herein, and a pharmaceutically acceptable carrier or diluent.

In another aspect, the present invention provides multimers comprising two or more multispecific molecules of the invention as well as multispecific carrier molecule comprising a plurality of first binding molecules and a plurality of second binding molecules, wherein each first binding molecule is a polypeptide comprising a single chain peptide-major histocompatibility complex (pMHC) fusion, and each second binding molecule is a polypeptide comprising an antigen-binding domain that specifically binds a T-cell surface molecule.

In some embodiments, multimerization of the multispecific molecules of the invention can be achieved, for example, using IgG, streptavidin, streptactin, polysaccharides, dextrans, micelles, liposomes, cells, polymers, beads and other types of solid support, or small organic molecules carrying reactive groups or carrying chemical motifs that can bind the multispecific molecules of the invention and other molecules. Multimerization can involve the use of one or more carriers and/or one or more scaffolds and/or one or more linkers connecting carrier to scaffold, carrier to carrier, scaffold to scaffold. In one embodiment, scaffolds can be comprised of organic molecules carrying reactive groups, capable of reacting with reactive groups on a multispecific molecule that is being multimerized. Non-limiting examples of useful small organic molecules include molecules of cyclic structure such as, e.g., functionalized cycloalkanes or functionalized aromatic ring structures.

In some embodiments, multimerization of the multispecific molecules can involve covalent or non-covalent interactions. Non-limiting examples of covalent interactions include, e.g., acylation such as amide formation, pyrazolone formation, isoxazolone formation; alkylation; vinylation; disulfide formation, addition to carbon-hetero multiple bonds (e.g., alkene formation by reaction of phosphonates with aldehydes or ketones; arylation; alkylation of arenes/hetarenes by reaction with alkyl boronates or enolethers), nucleophilic substitution using activation of nucleophiles (e.g., condensations; alkylation of aliphatic halides or tosylates with enolethers or enamines), and cycloadditions. Non-limiting examples of molecule pairs and molecules that can form non-covalent interactions include, e.g., streptavidin/biotin, avidin/biotin, antibody/antigen, DNA/DNA, DNA/PNA, DNA/RNA, PNA/PNA, LNA/DNA, leucine zipper e.g. Fos/Jun, IgG dimeric protein, IgM multivalent protein, acid/base coiled-coil helices, chelate/metal ion-bound chelate, streptavidin (SA) and avidin and derivatives thereof, biotin, immunoglobulins, antibodies (monoclonal, polyclonal, and recombinant), antibody fragments and derivatives thereof, leucine zipper domain of AP-1 (jun and fos), hexa-his (SEQ ID NO: 29) (metal chelate moiety), hexa-hat GST (glutathione S-transferase) glutathione affinity, Calmodulin-binding peptide (CBP), Strep-tag, Cellulose Binding Domain, Maltose Binding Protein, S-Peptide Tag, Chitin Binding Tag, Immunoreactive Epitopes, Epitope Tags, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope, lectins that mediate binding to a diversity of compounds, including carbohydrates, lipids and proteins, e.g. Con A (Canavalia ensiformis) or WGA (wheat germ agglutinin) and tetranectin or Protein A or G (antibody affinity). Combinations of such binding entities can also be used.

When multimerization involves the use of a solid surface (e.g., a bead or a plate comprising, e.g., glass, silica, polyesters of hydroxy carboxylic acids, polyanhydrides of dicarboxylic acids, or copolymers of hydroxy carboxylic acids and dicarboxylic acids), such surface can be covalently or non-covalently coated with the multimers or single multispecific molecules, through non-cleavable or cleavable linkers. As an example, the surface can be coated with streptavidin monomers, which in turn are associated with biotinylated multispecific molecules of the invention; or the surface can be coated with streptavidin tetramers, each of which are associated with 0, 1, 2, 3, or 4 biotinylated multispecific molecules of the invention; or the surface can be coated with molecule-dextramers where e.g. the reactive groups of the molecule-dextramer (e.g. the divinyl sulfone-activated dextran backbone) has reacted with nucleophilic groups on the surface, to form a covalent linkage between the dextran of the dextramer and the surface. The surface can further contain a flexible or rigid, and water soluble, linker that allows for the immobilized multispecific molecules to interact efficiently with T cells. In yet another embodiment, the linker is cleavable, allowing for release of the multispecific molecules from the surface. Non-limiting examples of linker molecules that may be employed in the present invention include Calmodulin-binding peptide (CBP), 6×HIS (SEQ ID NO: 29), Protein A, Protein G, biotin, Avidine, Streptavidine, Strep-tag, Cellulose Binding Domain, Maltose Binding Protein, S-Peptide Tag, Chitin Binding Tag, Immuno-reactive Epitopes, Epitope Tags, GST tagged proteins, E2Tag, HA Epitope Tag, Myc Epitope, FLAG Epitope, AU1 and AU5 Epitopes, Glu-Glu Epitope, KT3 Epitope, IRS Epitope, Btag Epitope, Protein Kinase-C Epitope, VSV Epitope.

In some embodiments, the multimerized multispecific molecule species are chemically cross-linked multispecific molecules of the invention (for example cross-linked to dendrimers) For example, the multispecific molecules of the invention can be genetically modified by including sequences encoding amino acid residues with chemically reactive side chains such as Cys or His. Such amino acids with chemically reactive side chains may be positioned in a variety of positions of a multispecific molecule (e.g., distal to the presenting peptide and binding domain of the pMHC complex). Suitable side chains can be used to chemically link two or more multispecific molecules of the invention to a suitable dendrimer particle to produce a multimerized molecule. Dendrimers are synthetic chemical polymers that can have any one of a number of different functional groups of their surface [D. Tomalia, Aldrichimica Acta, 26:91:101 (1993)]. Non-limiting examples of useful dendrimers include, e.g., a polyamidoamine, a polyamidoalcohol, a polyalkyleneimine, a polyalkylene, a polyether, a polythioether, a polyphosphonium, a polysiloxane, a polyamide, and a polyaryl polymer.

For additional disclosure of means for multimerization see, e.g., U.S. Pat. Nos. 8,268,964; 7,074,905; U.S. Pat. Appl. Pub. No. 20050003431.

In another aspect, the present invention provides a multispecific carrier comprising a plurality of first molecules for engaging T cell receptors and a plurality of second molecules for engaging T cell surface molecule(s) to modulate an activity of the T cell, wherein: each first molecule is a polypeptide comprising a peptide (p) presented in the context of a major histocompatibility complex (MHC) molecule (pMHC complex) or a fragment, mutant or derivative thereof, and each second molecule comprises a domain that specifically binds a molecule expressed on the surface of the cell expressing the TCR, and wherein the first and second molecules can be separate molecules which are not joined together to form a multispecific molecule. In certain embodiments, each first molecule of the plurality of first molecules is the same. In certain embodiments, each first molecule of the plurality of first molecules comprises at least two, three, four, five, or six different types of first molecules. In certain embodiments, each second molecule of the plurality of second molecules is the same. In certain embodiments, each second molecule of the plurality of second molecules comprises at least two, three, four, five, or six different types of first molecules.

In some embodiments of the multispecific carrier, the domain that specifically binds a molecule expressed on the surface of the cell expressing the TCR is an antigen-binding domain. In some embodiments, the antigen-binding domain is a monovalent antigen-binding domain. In certain embodiments, the antigen-binding domain binds to a T-cell immunomodulatory molecule (e.g., CD28 or CTLA4) on the surface of the cell expressing the TCR. In some embodiments, the domain that specifically binds a molecule expressed on the surface of the cell expressing the TCR is a small molecule, protein, fusion protein, peptide, aptamer, avimer or derivatives or fragments thereof. In certain embodiments, the domain that specifically binds a molecule expressed on the surface of the cell expressing the TCR is a T-cell immunomodulatory molecule, that upon binding the molecule on the surface of the cell expressing the TCR modulates the activity of the T cell (e.g., a co-stimulatory molecule such as CD80 or CD86 or a inhibitory molecule such as PD-L1 or B7-H3). In certain embodiments, the domain that specifically binds a molecule expressed on the surface of the cell expressing the TCR is a protein or derivative or fragment thereof, wherein the protein is an adhesion molecule (e.g., ICAM).

In some embodiments of the multispecific carrier, the MHC comprises (i) a class I MHC polypeptide or a fragment (e.g., the peptide binding groove), mutant or derivative thereof, and optionally, (ii) a β2 microglobulin polypeptide or a fragment, mutant or derivative thereof. In some embodiments, the MHC comprises α1 and α2 domains of a class I MHC polypeptide, or a fragment, mutant or derivative thereof. In some embodiments, the MHC comprises α1, α2, and α3 domains of a class I MHC polypeptide, or a fragment, mutant or derivative thereof. In some embodiments, MHC comprises the class I MHC polypeptide (or portion thereof) and β2 microglobulin. In some embodiments, the class I MHC (or portion thereof) and β2 microglobulin polypeptide are linked, e.g., by a peptide linker. In some embodiments, the class I MHC polypeptide is a human class I MHC polypeptide selected from the group consisting of HLA-A, HLA-B, HLA-C, HLA-E, HLA-F, and HLA-G. In another specific embodiment, the class I MHC polypeptide is a murine class I MHC polypeptide selected from the group consisting of H-2K, H-2D, H-2L, H-2Q, H-2M, and H-2T.

In some embodiments of the multispecific carrier, the MHC comprises a class II MHC polypeptide or a fragment, mutant or derivative thereof. In some embodiments, the MHC comprises α and β polypeptides of a class II MHC complex, or respective fragments, mutants or derivatives thereof. In some embodiments, the MHC comprises α1 and β1 domains of α and β polypeptides, respectively, of a class II MHC complex or a fragment, mutant or derivative thereof. In some embodiments, the α and β polypeptides (or fragments thereof, e.g., the α1 and β1 domains) are linked by a peptide linker. In one specific embodiment, the MHC comprises α and β polypeptides (or fragments thereof, e.g., the α1 and β1 domains) of a human class II MHC complex selected from the group consisting of HLA-DP, HLA-DR, HLA-DQ, HLA-DM and HLA-DO. In another specific embodiment, the MHC comprises α and β polypeptides (or fragments thereof, e.g., the α1 and β1 domains) of a murine H-2A or H-2E class II MHC complex.

In some embodiments of the multispecific carrier, the T-cell surface molecule to which the domain that specifically binds a molecule expressed on the surface of the cell expressing the TCR specifically binds is a T-cell co-stimulatory molecule. In some embodiments, the T-cell co-stimulatory molecule is selected from the group consisting of CD28, CD40L, ICOS, CD27, OX40, 4-1BB, GITR, HVEM, galectin 9, LFA-1, DR3, CD30 SLAM. 2B4, CD226, TIM1, TIM2, and CD2.

In some embodiments of the multispecific carrier, the T-cell surface molecule to which the domain that specifically binds a molecule expressed on the surface of the cell expressing the TCR specifically binds molecule is a T-cell inhibitory molecule. In some embodiments, the inhibitory molecule is selected from the group consisting of CTLA4, PD1, BTLA, TIM3, TIGIT, CD160, LAG3, LAIR1, MHC-1, B7-1 and B7-H1.

In various embodiments of the multispecific carrier, the peptide consists of about 5 to about 40 amino acid residues, from about 6 to about 30 amino acid residues, from about 8 to about 20 amino acid residues, or about 9, 10, or 11 amino acid residues.

In some embodiments, the peptide is derived from a viral antigen or a bacterial antigen. In some embodiments, the viral antigen is derived from a virus selected from the group consisting of adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, and zika, or the bacterial antigen is derived from a bacterium selected from the group consisting of methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, and Clindamycin-resistant group B Streptococcus.

In some embodiments, the peptide is derived from a tumor-associated antigen. In some embodiments, the tumor-associated antigen is selected from the group consisting of adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML 1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1, -A2, -A3, -A4, -A6, -A9, -A10, -A12, -C1, and C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, WT-1. In some embodiments, the peptide is a neo-antigen. In some embodiments, the peptide is a tumor specific antigen.

In some embodiments, the peptide is derived from an antigen associated with an autoimmune disorder. In some embodiments, the antigen is selected from the group consisting of gliadin (celiac disease; e.g., (i) α-gliadin fragment corresponding to amino acids 57-73 or (ii) γ-gliadin fragment corresponding to amino acids 139 153 or (iii) ω-gliadin fragment corresponding to amino acids 102-118), GAD 65, IA-2 and insulin β chain (for type 1-diabetes), glatiramer acetate (GA) (for multiple sclerosis), acetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)).

In certain embodiments of the multispecific carrier, the carrier is a cell. In certain embodiments, the cell is a cell line such as, but not limited to, CHO, HEK 293 (embryonic kidney), HMEC (epithelial), HIVE-55 (endothelial), HIVS-125 (smooth muscle), and tumor cells (e.g. HN5). In certain embodiments, the cell is an antigen presenting cell. In certain embodiments, the antigen presenting cell is a macrophage or a dendritic cell. In certain embodiments, the cell is a B-cell.

In certain embodiments of the multispecific carrier, the multispecific carrier is a virus-like particle.

In another aspect, the present invention provides a pharmaceutical composition comprising a multispecific carrier as discussed above or herein and a pharmaceutically acceptable carrier.

In another aspect, the present invention provides a set or series of nucleic acid molecules encoding (i) a first transmembrane polypeptide and (ii) a second transmembrane polypeptide, wherein the first transmembrane polypeptide comprises a first extracellular domain comprising a single chain peptide-major histocompatibility complex (pMHC) fusion, and a first transmembrane domain, and wherein the second transmembrane polypeptide comprises a second extracellular domain comprising an antigen-binding domain that specifically binds a T-cell surface molecule, and a second transmembrane domain. In some embodiments, the antigen-binding domain comprises a single-chain variable fragment (scFv) domain comprising a light chain variable region (LCVR) and a heavy chain variable region (HCVR), and wherein the scFv is connected directly or through a linker to the second extracellular domain.

In some embodiments of the nucleic acid molecules, the pMHC complex comprises a class I MHC polypeptide. In some embodiments, the pMHC complex comprises a peptide, a β₂-microglobulin peptide, and a class I alpha chain domain or fragment thereof. In some embodiments, the pMHC complex comprises, from N-terminus to C-terminus, a peptide, a β2-microglobulin peptide, and a class I alpha chain domain or fragment thereof. In some embodiments, the pMHC complex comprises, from N-terminus to C-terminus, a peptide, an optional first linker, a β₂-microglobulin peptide, an optional second linker, and class I MHC alpha chain domains 1, 2 and/or 3.

In some embodiments of the nucleic acid molecules, the pMHC complex comprises a class II MHC polypeptide. In some embodiments, the pMHC complex comprises a peptide, a class II MHC alpha chain domain or a fragment thereof, and a class II MHC beta chain domain or a fragment thereof. In some embodiments, the MHC class II alpha chain consists of the extracellular domains of the class II alpha chain. In some embodiments, the MHC class II beta chain consists of the extracellular domains of the class II beta chain. In some embodiments, the pMHC complex comprises, from N-terminus to C-terminus, a peptide, class II MHC alpha chain extracellular domains, and class II MHC beta chain extracellular domains. In some embodiments, the pMHC complex comprises, from N-terminus to C-terminus, a peptide, an optional first linker, class II MHC alpha chain domains 1 and 2, an optional second linker, and class II MHC beta chain domains 1 and 2.

In some embodiments of the nucleic acid molecules, the T-cell surface molecule is a T-cell co-stimulatory molecule. In some embodiments, the T-cell co-stimulatory molecule is selected from the group consisting of CD28, CD40L, ICOS, CD27, OX40, 4-1BB, GITR, HVEM, galectin 9, LFA-1, DR3, CD30 SLAM. 2B4, CD226, TIM1, TIM2, and CD2.

In some embodiments of the nucleic acid molecules, the T-cell surface molecule is a T-cell inhibitory molecule. In some embodiments, the inhibitory molecule is selected from the group consisting of CTLA4, PD1, BTLA, TIM3, TIGIT, CD160, LAG3, LAIR1, MHC-1, B7-1 and B7-H1.

In various embodiments of the nucleic acid molecules, the peptide consists of about 5 to about 40 amino acid residues, from about 6 to about 30 amino acid residues, from about 8 to about 20 amino acid residues, or about 9, 10, or 11 amino acid residues.

In some embodiments of the nucleic acid molecules, the peptide is derived from a viral antigen or a bacterial antigen. In some embodiments, the viral antigen is derived from a virus selected from the group consisting of adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, and zika, or the bacterial antigen is derived from a bacterium selected from the group consisting of methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, and Clindamycin-resistant group B Streptococcus.

In some embodiments of the nucleic acid molecules, the peptide is derived from a tumor-associated antigen. In some embodiments, the tumor-associated antigen is selected from the group consisting of adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1, -A2, -A3, -A4, -A6, -A9, -A10, -A12, -C1, and -C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, WT-1. In some embodiments, the peptide is a neo-antigen. In some embodiments, the peptide is a tumor specific antigen.

In some embodiments of the nucleic acid molecules, the peptide is derived from an antigen associated with an autoimmune disorder. In some embodiments, the antigen is selected from the group consisting of gliadin (celiac disease; e.g., (i) α-gliadin fragment corresponding to amino acids 57-73 or (ii) γ-gliadin fragment corresponding to amino acids 139 153 or (iii) ω-gliadin fragment corresponding to amino acids 102-118), GAD 65, IA-2 and insulin β chain (for type 1-diabetes), glatiramer acetate (GA) (for multiple sclerosis), acetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)).

In another aspect, the present invention provides a vector or vectors comprising any of the nucleic acid molecules discussed above or herein. In some embodiments, the vector or vectors is a DNA vector, an RNA vector, a plasmid, a lentivirus vector, an adenovirus vector, or a retroviral vector. In some embodiments, the vector or vectors is a lentiviral vector.

In another aspect, the present invention provides a method of modulating T-cell activity in a subject, comprising administering to the subject a multispecific molecule or a multispecific carrier as discussed above or herein, whereby administration of the multispecific molecule or multispecific carrier modulates activation, proliferation and/or survival of T-cells.

In some embodiments of the method, the pMHC complex comprises a class I MHC polypeptide, and wherein the multispecific molecule or the multispecific carrier molecule modulates activation, proliferation and/or survival of CD8⁺ T-cells.

In some embodiments of the method, the T-cell surface molecule is a T-cell co-stimulatory molecule, and whereby administration of the multispecific molecule or the multispecific carrier molecule stimulates CD8⁺ T-cell activation, proliferation and/or survival. In some embodiments, the T-cell co-stimulatory molecule is selected from the group consisting of CD28, ICOS, HVEM, CD27, 4-1BB, 0X40, DR3, GITR, CD30, SLAM, CD2, 2B4, TIM1, TIM2, and CD226.

In some embodiments of the method, the T-cell surface molecule is a T-cell inhibitory molecule, and whereby administration of the multispecific molecule or the multispecific carrier molecule inhibits CD8⁺ T-cell activation, proliferation and/or survival. In some embodiments, the inhibitory molecule is selected from the group consisting of CTLA4, PD1, BTLA, TIM3, CD160, LAG3, LAIR1, B7-1 and B7-H1. In some embodiments, the inhibition of CD8⁺ T-cell activation, proliferation and/or survival results in induction of T cell anergy or T cell death.

In some embodiments of the method, the pMHC complex comprises a peptide and a class II MHC polypeptide, and wherein the multispecific molecule or the multispecific carrier molecule modulates activation, proliferation and/or survival of CD4⁺ T-cells.

In some embodiments of the method, the T-cell surface molecule is a T-cell co-stimulatory molecule, and whereby administration of the multispecific molecule or the multispecific carrier molecule stimulates CD4⁺ T-cell activation, proliferation and/or survival. In some embodiments, the T-cell co-stimulatory molecule is selected from the group consisting of CD28, ICOS, HVEM, CD27, 4-1BB, 0X40, DR3, GITR, CD30, SLAM, CD2, 2B4, CD226, TIM1 and TIM2.

In some embodiments of the method, the T-cell surface molecule is a T-cell inhibitory molecule, and whereby administration of the multispecific molecule or the multispecific carrier molecule inhibits CD4⁺ T-cell activation, proliferation and/or survival. In some embodiments, the inhibitory molecule is selected from the group consisting of CTLA4, PD1, BTLA, TIM3, TIGIT, CD160, LAG3, LAIR1, B7-1 and B7-H1. In some embodiments, the inhibition of CD4⁺ T-cell activation, proliferation and/or survival results in induction of T cell anergy or T cell death.

In another aspect, the present invention provides a method of treating an infection in a subject, comprising administering to the subject a multispecific molecule or a multispecific carrier molecule comprising a class I MHC protein as discussed above or herein, wherein the antigen-binding domain specifically binds a T-cell co-stimulatory molecule. In some embodiments, the antigen-binding domain specifically binds CD28, CD40L, ICOS, CD27, OX40, 4-1BB, GITR, HVEM, galectin 9, LFA-1, DR3, CD30 SLAM. 2B4, CD226, TIM1, TIM2, and CD2.

In another aspect, the present invention provides a method of treating an infection in a subject, comprising administering to the subject a multispecific molecule or a multispecific carrier molecule comprising a class II MHC protein as discussed above or herein, wherein the antigen-binding domain specifically binds a T-cell co-stimulatory molecule. In some embodiments, the antigen-binding domain specifically binds CD28, CD40L, ICOS, CD27, OX40, 4-1BB, GITR, HVEM, galectin 9, LFA-1, DR3, CD30 SLAM. 2B4, CD226, TIM1, TIM2, and CD2.

In various embodiments of the methods of treating an infection, the agent causing the infection is a virus, and the peptide is a fragment of a viral protein. In some embodiments, the virus may be any one of the viruses discussed above or herein.

In another aspect, the present invention provides a method of treating a cancer in a subject, comprising administering to the subject a multispecific molecule or a multispecific carrier molecule comprising a class I MHC protein as discussed above or herein, wherein the antigen-binding domain specifically binds a T-cell co-stimulatory molecule. In some embodiments, the antigen-binding domain specifically binds CD28, CD40L, ICOS, CD27, OX40, 4-1BB, GITR, HVEM, galectin 9, LFA-1, DR3, CD30 SLAM. 2B4, CD226, TIM1, TIM2, and CD2.

In another aspect, the present invention provides a method of treating a cancer in a subject, comprising administering to the subject a multispecific molecule or a multispecific carrier molecule comprising a class II MHC protein as discussed above or herein, wherein the antigen-binding domain specifically binds a T-cell co-stimulatory molecule. In some embodiments, the antigen-binding domain specifically binds CD28, CD40L, ICOS, CD27, OX40, 4-1BB, GITR, HVEM, galectin 9, LFA-1, DR3, CD30 SLAM. 2B4, CD226, TIM1, TIM2, and CD2.

In another aspect, the present invention provides a method of treating an autoimmune disorder in a subject, comprising administering to the subject a multispecific molecule or a multispecific carrier molecule comprising a class I MHC protein as discussed above or herein, wherein the antigen-binding domain specifically binds a T-cell inhibitory molecule. In some embodiments, the antigen-binding domain specifically binds CTLA4, PD1, BTLA, TIM3, CD160, LAG3, LAIR1, B7-1 and B7-H1.

In another aspect, the present invention provides a method of treating an autoimmune disorder in a subject, comprising administering to the subject a multispecific molecule or a multispecific carrier molecule comprising a class II MHC protein as discussed above or herein, wherein the antigen-binding domain specifically binds a T-cell inhibitory molecule. In some embodiments, the antigen-binding domain specifically binds CTLA4, PD1, BTLA, TIM3, TIGIT, CD160, LAG3, LAIR1, B7-1 and B7-H1.

In another aspect, the present invention provides a method of treating an infection in a subject, comprising administering to the subject an engineered cell as discussed above or herein, wherein the antigen-binding domain specifically binds a T-cell co-stimulatory molecule. In some embodiments, the antigen-binding domain specifically binds CD28, ICOS, HVEM, CD27, 4-1BB, 0X40, DR3, GITR, CD30, SLAM, CD2, 2B4, CD226, TIM1, or TIM2. In some embodiments, the agent causing the infection is a virus, and the peptide is a fragment of a viral protein.

In another aspect, the present invention provides a method of treating a cancer in a subject, comprising administering to the subject an engineered cell as discussed above or herein, wherein the antigen-binding domain specifically binds a T-cell co-stimulatory molecule. In some embodiments, the antigen-binding domain specifically binds CD28, ICOS, HVEM, CD27, 4-1BB, 0X40, DR3, GITR, CD30, SLAM, CD2, 2B4, CD226, TIM1, or TIM2.

In another aspect, the present invention provides a method of treating an autoimmune disorder in a subject, comprising administering to the subject an engineered cell as discussed above or herein, wherein the antigen-binding domain specifically binds a T-cell inhibitory molecule. In some embodiments, the antigen-binding domain specifically binds CTLA4, PD1, BTLA, TIM3, CD160, LAG3, LAIR1, B7-1, or B7-H1.

In another aspect, the present invention provides a method of modulating an activity of T-cells ex vivo, comprising: obtaining CD8⁺ T-cells from a subject; and culturing the CD8⁺ T-cells with a plurality of multispecific molecules or multispecific carrier molecules under conditions and for a period of time sufficient to modulate the activity of the CD8⁺ T-cells, wherein each of the plurality of multispecific molecules or multispecific carrier molecules comprises a class I MHC protein as discussed above or herein, and whereby the activity of CD8⁺ T-cells having a T-cell receptor (TCR) with specificity for the peptide is modulated.

In some embodiments of the method, the T-cell surface molecule is a T-cell co-stimulatory molecule, and the CD8⁺ T-cells having a TCR with specificity for the peptide are activated and/or proliferate. In some embodiments, the T-cell co-stimulatory molecule is selected from the group consisting of CD28, ICOS, HVEM, CD27, 4-1BB, 0X40, DR3, GITR, CD30, SLAM, CD2, 2B4, TIM1, TIM2, and CD226.

In some embodiments of the method, the T-cell surface molecule is a T-cell inhibitory molecule, and the CD8⁺ T-cells having a TCR with specificity for the peptide are de-activated. In some embodiments, the inhibitory molecule is selected from the group consisting of CTLA4, PD1, BTLA, TIM3, CD160, LAG3, LAIR1, B7-1 and B7-H1.

In some embodiments of the method, the CD8⁺ T-cells are tumor infiltrating lymphocytes.

In another aspect, the present invention provides a method of modulating an activity of T-cells ex vivo, comprising: obtaining CD4⁺ T-cells from a subject; and culturing the CD4⁺ T-cells with a plurality of multispecific molecules or multispecific carrier molecules under conditions and for a period of time sufficient to modulate the activity of the CD4⁺ T-cells, wherein each of the plurality of multispecific molecules or multispecific carrier molecules comprises a class II MHC protein as discussed above or herein, and whereby the activity of CD4⁺ T-cells having a T-cell receptor (TCR) with specificity for the peptide is modulated.

In some embodiments of the method, the T-cell surface molecule is a T-cell co-stimulatory molecule, and the CD4⁺ T-cells having a TCR with specificity for the peptide are activated and/or proliferate. In some embodiments, the T-cell co-stimulatory molecule is selected from the group consisting of CD28, ICOS, HVEM, CD27, 4-1BB, 0X40, DR3, GITR, CD30, SLAM, CD2, 2B4, CD226, TIM1 and TIM2.

In some embodiments of the method, the T-cell surface molecule is a T-cell inhibitory molecule, and the CD4⁺ T-cells having a TCR with specificity for the peptide are de-activated. In some embodiments, the inhibitory molecule is selected from the group consisting of CTLA4, PD1, BTLA, TIM3, TIGIT, CD160, LAG3, LAIR1, B7-1 and B7-H1.

In various embodiments of the ex vivo methods discussed above, the plurality of multispecific molecules are bound to a scaffold in a clustered arrangement. In some embodiments, the plurality of multispecific molecules are clustered via one or more linkers. In some embodiments, the one or more linkers is a multivalent antibody that binds the first and/or the second Fc domain of the multispecific molecules. In some embodiments, the ratio of multivalent antibodies to multispecific molecules is about 1:1 in the culture.

In another aspect, the present invention provides a method of treating or ameliorating a disease or disorder in which T-cells modulated by the ex vivo methods discussed above are reintroduced into the subject. In some embodiments, the disease or disorder is an infection, a cancer, or an autoimmune disorder.

In another aspect, the present invention provides a method of treating or ameliorating an infection (e.g., a viral infection or a bacterial infection) or a cancer in a subject, comprising: (a) obtaining CD8⁺ T-cells from a subject; (b) culturing the CD8⁺ T-cells with a plurality of multispecific molecules or multispecific carrier molecules under conditions and for a period of time sufficient to activate and/or proliferate the CD8⁺ T-cells, wherein each of the plurality of multispecific molecules or multispecific carrier molecules comprises a class I MHC protein as discussed above or herein; and (c) reintroducing activated and/or proliferated CD8⁺ T-cells that have a TCR with specificity for the peptide into the subject, whereby the viral infection or cancer is treated or ameliorated.

In another aspect, the present invention provides a method of treating or ameliorating an autoimmune disorder in a subject, comprising: (a) obtaining CD4⁺ T-cells from a subject; (b) culturing the CD4⁺ T-cells with a plurality of multispecific molecules or multispecific carrier molecules under conditions and for a period of time sufficient to inactivate the CD4⁺ T-cells, wherein each of the plurality of multispecific molecules or multispecific carrier molecules comprises a class II MHC protein as discussed above or herein; and (c) reintroducing inactivated CD4⁺ T-cells that have a TCR with specificity for the peptide into the subject, whereby the autoimmune disorder is treated or ameliorated.

In another aspect, the present invention provides for the use of the multispecific molecules discussed above or herein for the treatment and/or prevention of infections (e.g., viral infections), cancer, and/or autoimmune disorders. In other aspects, the present invention provides for the use of the multispecific molecules, multispecific carrier molecules, or engineered cells discussed above or herein in the manufacture of a medicament for treating and/or preventing an infection, cancer and/or an autoimmune disorder.

Other embodiments will become apparent from a review of the detailed description and the accompanying drawings. Any reference to a multispecific molecule above or herein refers also to a bispecific molecule.

### BRIEF DESCRIPTION OF THE DRAWINGS

FIG. 1 illustrates non-limiting embodiments of two plasmids for the production of an exemplary multispecific molecule in accordance with an embodiment of the present invention (as discussed in Example 1), and a non-limiting illustration of an embodiment of the multispecific molecule comprising a first molecule having a peptide-MHC single chain fusion and an immunoglobulin Fc domain (including a modified CH3 domain to facilitate purification of the multispecific molecule), and a second molecule having an antigen-binding domain with specificity for CD28 and an immunoglobulin Fc domain.

FIG. 2 illustrates the gating strategy for the enrichment of CD8+ T cells from C57BL6 splenocytes via flow cytometry, as discussed in Example 2. Enrichment increased the percentage of CD8+ T cells in the population from 6.4% to 91.0%.

FIG. 3 illustrates the gating strategy for the assessment of the proliferation of isolated peptide (GP-33)-specific CD8+ T cells stimulated with plate bound multispecific GP-33-MHC×anti-CD28 molecules, as discussed in Example 3. Quadrant 1 (Q1) of each of the two right panels (labeled Plate bound scMHC/gp33×anti-CD28 and Unstimulated cells) shows the percentage of proliferating CD8+ T cells specific for the peptide (GP-33), while Q3 of each panel shows the percentage of non-proliferating, non-peptide-specific CD8+ T cells.

FIG. 4 illustrates the effect of cytokines (IL-2 and IL-7) on the expansion of peptide (GP-33)-specific CD8+ T cells with or without stimulation with plate bound multispecific GP-33-MHC×anti-CD28 molecules, as discussed in Example 4. Q1 of each panel shows the percentage of proliferating CD8+ T cells specific for the peptide (GP-33), while Q3 of each panel shows the percentage of non-proliferating, non-peptide-specific CD8+ T cells.

FIG. 5 illustrates the effects of the ratio of a cross-linking polyclonal antibody to multispecific molecule on the proliferation of peptide (GP-33)-specific CD8+ T cells relative to the absence of crosslinker. Panels showing the effects of ratios of 5:1, 4:1, 3:1, 2:1, 1:1, 1:2, 1:3, 1:4 and 1:5 (crosslinking reagent:multispecific) are shown along with a panel showing the absence of crosslinker. Q1 of each panel shows the percentage of proliferating CD8+ T cells specific for the peptide (GP-33), while Q3 of each panel shows the percentage of non-proliferating, non-peptide-specific CD8+ T cells.

FIG. 6 illustrates the effects of varying concentrations of HEK293 cell-bound multispecific GP33-MHC×anti-CD28 molecules with an IgG1 Fc domain on the proliferation of peptide (GP-33)-specific CD8+ T cells, as discussed in Example 6. Q1 of each panel shows the percentage of proliferating CD8+ T cells specific for the peptide (GP-33), while Q3 of each panel shows the percentage of non-proliferating, non-peptide-specific CD8+ T cells. The results are also illustrated in the accompanying bar graphs.

FIG. 7 illustrates the effects of varying concentrations of HEK293 cell-bound multispecific GP33-MHC×anti-CD28 molecules with an IgG4 Fc domain on the proliferation of peptide (GP-33)-specific CD8+ T cells, as discussed in Example 7. Q1 of each panel shows the percentage of proliferating CD8+ T cells specific for the peptide (GP-33), while Q3 of each panel shows the percentage of non-proliferating, non-peptide-specific CD8+ T cells. The results are also illustrated in the accompanying bar graphs.

FIG. 8 illustrates the effects of varying concentrations of cell (CD20+ B cells)-bound multispecific GP33-MHC×anti-CD28 molecules with an IgG4 Fc domain and a C-terminal anti-CD20 scFv on the proliferation of peptide (GP-33)-specific CD8+ T cells, compared to the presence of CD20-Jurkat cells, as discussed in Example 8. Q1 of each panel shows the percentage of proliferating CD8+ T cells specific for the peptide (GP-33), while Q3 of each panel shows the percentage of non-proliferating, non-peptide-specific CD8+ T cells. The results are also illustrated in the accompanying bar graphs.

FIG. 9 illustrates the effects of plate-bound or cell (CD20+ B cells)-bound multispecific GP33-MHC×anti-CD28 molecules with an IgG4 Fc domain and a C-terminal anti-CD20 scFv on the number of cell divisions of peptide (GP-33)-specific CD8+ T cells, as discussed in Example 9. Comparisons against non-bound (soluble and CD20-Jurkat cells) multispecific molecules are also provided. Q1 of each upper panel shows the percentage of dividing CD8+ T cells specific for the peptide (GP-33), while Q3 of each panel shows the percentage of non-dividing, non-peptide-specific CD8+ T cells. The percentage of maximum proliferation for each experiment (with or without the multispecific molecule) is also illustrated in the lower panels.

FIG. 10 illustrates the effects of varying numbers of virus-like particles (VLPs) arrayed with scMHCgp33 and anti-CD28 molecules on the proliferation of peptide (GP-33)-specific CD8+ T cells, compared to VLPs arrayed with a control peptide (smMHCova257) and anti-CD28 molecules, as discussed in Example 10. Q1 of each panel shows the percentage of proliferating CD8+ T cells specific for the peptide (GP-33), while Q3 of each panel shows the percentage of non-proliferating, non-peptide-specific CD8+ T cells. The results are also illustrated in the accompanying bar graph.

FIG. 11 illustrates the effects of engineered primary B cells expressing scMHCp proteins on the activation and proliferation of Ag-specific CD8+ T cells in vitro, as discussed in Example 11. Q1 of each panel shows the percentage of proliferating CD8+ T cells specific for the antigen (GP-33 or OVA), while Q3 of each panel shows the percentage of non-proliferating, non-peptide-specific CD8+ T cells.

FIG. 12 illustrates the effects of engineered primary B cells expressing scMHCp proteins on the activation and proliferation of Ag-specific OTI CD8+ T cells in vivo, as discussed in Example 12. OTI CD8+ T cells in each tissue compartment tested (Blood, Lymph Node, Spleen) proliferated (i.e. dilution of proliferation dye) in response to scMHCova retrovirus (RV) engineered B cells and not to irrelevant scMHCgp33 RV engineered B cells indicating successful in vivo T cell activation.

FIG. 13 illustrates the effect of in vivo delivered scMHCp modified primary B cells or scMHCp VLPs on the generation of lytic function in OTI CD8⁺ T cells, as discussed in Example 13. Mice were adoptively transferred with OTI CD8 T cells and subsequently treated with RV scMHCp B cells or with scMHCp/antiCD28 VLPs. scMHCova RV B cells and scMHCova VLPs enhanced ova-specific killing over background similar to control ova257 peptide loaded B cells and ova257/CFA immunization as depicted in bottom right bar graph.

FIG. 14 illustrates the effect of in vivo delivered scMHCova Stahl body on the proliferation of OTI CD8⁺ T cells, as discussed in Example 14. Mice were adoptively transferred with OTI CD8⁺ T cells and subsequently treated with scMHCova or scMHCgp33 Stahl bodies. OTI cells from mice proliferated (i.e. dilution of proliferation dye) in response to treatment with scMHCova Stahl body while OTI cells in mice treated with irrelevant control scMHCgp33 Stahlbody did not.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims along with the full scope of equivalents to which those claims are entitled.

Unless defined otherwise, all technical and scientific terms used herein have the same meaning as commonly understood by one of ordinary skill in the art to which this invention belongs. As used herein, the term “about,” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art. For example, as used herein, the expression “about 100” includes 99 and 101 and all values in between (e.g., 99.1, 99.2, 99.3, 99.4, etc.).

“Optional” or “optionally” means that the subsequently described event or circumstance may or may not occur and that the description includes instances in which the event or circumstance occurs and instances in which it does not.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims along with the full scope of equivalents to which those claims are entitled.

All patents, applications and non-patent publications mentioned in this specification are incorporated herein by reference in their entireties.

### Definitions

All references to proteins, polypeptides and protein fragments herein are intended to refer to the human version of the respective protein, polypeptide or protein fragment unless explicitly specified as being from a non-human species. For example, the expression “CD28” means human CD28 unless specified identified as being from a non-human species, e.g., “mouse CD28,” “monkey CD28,” etc.

The term “T-cell,” as used herein, refers to all types of immune cells expressing CD3, including CD4⁺ cells (helper T cells), CD8⁺ cells (cytotoxic T cells), regulatory T cells (Tregs), natural killer cells, and tumor infiltrating lymphocytes.

The term “antigen” refers to any agent (e.g., protein, peptide, polysaccharide, glycoprotein, glycolipid, nucleic acid, portions thereof, or combinations thereof) that, when introduced into a host, animal or human, having an immune system (directly or upon expression as in, e.g., DNA vaccines), is recognized by the immune system of the host and is capable of eliciting an immune response.

As described herein, the T cell receptor (TCR) recognizes a peptide presented in the context of a major histocompatibility complex (MHC) molecule. The peptide MHC (pMHC) complex is recognized by TCR, with the peptide (antigenic determinant) and the TCR idiotype providing the specificity of the interaction. Accordingly, the term “antigen” encompasses peptides presented in the context of MHC molecules. The peptide displayed on an MHC molecule may also be referred to as an “epitope” or an “antigenic determinant”. The terms “peptide,” “antigenic determinant” and “epitope” as used herein encompass not only those presented naturally by antigen-presenting cells (APCs) but may be any desired peptide so long as it is recognized by an immune cell, e.g., when presented appropriately to the cells of an immune system. For example, a peptide having an artificially prepared amino acid sequence may also be used as the epitope.

The terms “Major Histocompatibility Complex”, “MHC” and “MHC molecule” encompass naturally occurring MHC molecules as well as individual chains of MHC molecules (e.g., MHC class I α (heavy) chain, β2-microglobulin, MHC class II α chain, MHC class II βchain), individual subunits of such chains of MHC molecules (e.g., α1, α2, and/or α3 subunits of MHC class I α chain, α1 and/or α2 subunits of MHC class II α chain, β1 and/or β2 subunits of MHC class II β chain) as well as fragments, mutants and various derivatives thereof (including fusions proteins), wherein such fragments, mutants and derivatives retain the ability to display an antigenic peptide for recognition by a TCR, e.g., an antigen-specific TCR. An MHC class I molecule comprises a peptide binding groove formed by the α1 and α2 domains of the heavy α chain that can stow a peptide of around 8-10 amino acids. Despite the fact that both classes of MHC bind a core of about 9 amino acids within peptides, the open-ended nature of MHC class II peptide-binding groove (the α1 domain of a class II MHC α polypeptide in association with the β1 domain of a class II MHC β polypeptide) allows for a wider range of peptide lengths. Peptides binding MHC class II usually vary between 13 and 17 amino acids in length, though shorter or longer lengths are not uncommon. As a result, peptides may shift within the MHC class II peptide-binding groove, changing which 9-mer sits directly within the groove at any given time. Conventional identifications of particular MHC variants are used herein. For example, HLA-B 17 refers to a human leucocyte antigen from the B gene group (hence a class I type MHC) gene position (known as a gene locus) number 17; gene HLA-DR11, refers to a human leucocyte antigen coded by a gene from the DR region (hence a class II type MHC) locus number 11.

An antigenic determinant comprised in the multispecific molecules described herein can comprise any peptide that is capable of binding to an MHC protein in a manner such that the pMHC complex can bind to a TCR, preferably in a specific manner. In certain embodiments, such binding induces a T cell response. Examples include peptides produced by hydrolysis and most typically, synthetically produced peptides, including randomly generated peptides, specifically designed peptides, and peptides where at least some of the amino acid positions are conserved among several peptides and the remaining positions are random.

In nature, peptides that are produced by hydrolysis undergo hydrolysis prior to binding of the antigen to an MHC protein. Class I MHC typically present peptides derived from proteins actively synthesized in the cytoplasm of the cell. In contrast, class II MHC typically present peptides derived either from exogenous proteins that enter a cell's endocytic pathway or from proteins synthesized in the ER. Intracellular trafficking permits a peptide to become associated with an MHC protein.

The binding of a peptide to an MHC peptide binding groove can control the spatial arrangement of MHC and/or peptide amino acid residues recognized by a TCR. Such spatial control is due in part to hydrogen bonds formed between a peptide and an MHC protein. Based on the knowledge on how peptides bind to various MHCs, the major MHC anchor amino acids and the surface exposed amino acids that are varied among different peptides can be determined.

Preferably, the length of an MHC-binding peptide is from about 5 to about 40 amino acid residues, more preferably from about 6 to about 30 amino acid residues, and even more preferably from about 8 to about 20 amino acid residues, and even more preferably between about 9 and 11 amino acid residues, including any size peptide between 5 and 40 amino acids in length, in whole integer increments (i.e., 5, 6, 7, 8, 9 . . . 40). While naturally MHC Class II-bound peptides vary from about 9-40 amino acids, in nearly all cases the peptide can be truncated to an about 9-11 amino acid core without loss of MHC binding activity or T cell recognition.

Peptides include peptides comprising at least a portion, e.g., an antigenic determinant, of a protein selected from a group consisting of a self protein associated with an autoimmune disorder, proteins of infectious agents, and tumor associated proteins.

Non-limiting examples of self proteins associated with an autoimmune disorder include, e.g., gliadin (celiac disease; e.g., (i) α-gliadin fragment corresponding to amino acids 57-73 or (ii) γ-gliadin fragment corresponding to amino acids 139 153 or (iii) ω-gliadin fragment corresponding to amino acids 102-118), GAD 65, IA-2 and insulin β chain (for type 1-diabetes), glatiramer acetate (GA) (for multiple sclerosis), acetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)).

In certain embodiments, the antigen comprises a peptide (e.g., antigenic determinant of a protein) that is the target of an autoreactive T cell involved in celiac disease. For example, the peptide can be derived from, comprises a portion of gluten peptides, such as α-gliadin, γ-gliadin, and/or glutenins. In certain embodiments, the epitope is derived from α-gliadin (33-mer (57-89) and its truncated forms, 25-mer (64-89), 18-mer (71-89), 17-mer (57-73), 13-mer (57-68), and glia-20); from γ-gliadin (DQ2-γ-I, DQ2-γ-II, DQ2-γ-III, DQ2-γ-IV, and DQ2-γ-V, 14-mer-1 (105-118) and 14-mer-2 (173-186)); glutenin (Glt-19-39 and glt-156 (42-56)); and/or glu-5. In certain embodiments, the antigen-derived peptide can include α-gliadin (57-73), γ-gliadin (139-153), and/or ω-gliadin (102-118). See, e.g., Camarca et al., Endocrine, Metabolic & Immune Disorders-Drug Targets, 12:207-219 (2012); Camarca et al., J. Immunol., 182(7): 4158-4166 (2009).

In certain embodiments, the peptide comprises an antigenic determinant of a protein that is the target of an autoreactive T cell involved in psoriasis, e.g., BV3 and/or BV13S1.

In certain embodiments, the peptide comprises an antigenic determinant of a protein that is the target of an autoreactive T cell involved in multiple sclerosis, e.g., BV5S2, BV6S5, and/or BV13SI.

In certain embodiments, the peptide comprises an antigenic determinant of a protein that is the target of an autoreactive T cell involved in rheumatoid arthritis, e.g., BV3, BV14, and/or BV17.

Non-limiting examples of viral proteins from which peptides may be derived to be used in the multispecific molecules described herein include LCMV gp33, CMV pp65, HIV gag, EBV BMLF1 as well as antigens derived from influenza virus (e.g., surface glycoproteins hemagglutinin (HA) and neuramimidase (NA)); immunodeficiency virus (e.g., a human immunodeficiency virus antigens (HIV) such as gp120, gp160, p18 antigen Gag p17/p24, Tat, Pol, Nef, and Env); herpesvirus (e.g., a glycoprotein from herpes simplex virus (HSV), Marek's Disease Virus, cytomegalovirus (CMV), or Epstein-Barr virus); hepatitis virus (e.g., Hepatitis B surface antigen (HBsAg)); papilloma virus; rous associated virus (e.g., RAV-1 env); infectious bronchitis virus (e.g., matrix and/or preplomer); flavivirus (e.g., a Japanese encephalitis virus (JEV) antigen, a Yellow Fever antigen, or a Dengue virus antigen); Morbillivirus (e.g., a canine distemper virus antigen, a measles antigen, or rinderpest antigen such as HA or F); rabies (e.g., rabies glycoprotein G); parvovirus (e.g., a canine parvovirus antigen); poxvirus (e.g., an ectromelia antigen, a canary poxvirus antigen, or a fowl poxvirus antigen); chicken pox virus (varicella zoster antigen); infectious bursal disease virus (e.g., VP2, VP3, or VP4); Hantaan virus, and mumps virus.

Non-limiting examples of bacterial proteins which may be a source of bacterial peptides, e.g., antigenic determinants, that can be used in the multispecific molecules described herein include lipopolysaccharides isolated from gram-negative bacterial cell walls and staphylococcus-specific, streptococcus-specific, pneumococcus-specific (e.g., PspA; see PCT Publication No. WO 92/14488), Neisseria gonorrhea-specific, Borrelia-specific (e.g., OspA, OspB, OspC of Borrelia associated with Lyme disease such as Borrelia burgdorferi, Borrelia afzelli, and Borrelia garinii [see, e.g., U.S. Pat. No. 5,523,089; PCT Publication Nos. WO 90/04411, WO 91/09870, WO 93/04175, WO 96/06165, WO93/08306; PCT/US92/08697; Bergstrom et al., Mol. Microbiol., 1999; 3:479486; Johnson et al., Infect, and Immun. 1992; 60:1845-1853; Johnson et al., Vaccine 1995; 13:1086-1094; The Sixth International Conference on Lyme Borreliosis: Progress on the Development of Lyme Disease Vaccine, Vaccine 1995; 13:133-135]), and pseudomonas-specific proteins or peptides. Additional non-limiting examples of bacterial antigens include, e.g., antigens from Neisseria gonorrhea, Mycobacterium tuberculosis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Escherichia coli, Actinobacillus equuli, Salmonella abortus ovis, Salmonella abortus equi, Pseudomonas aeruginosa, Corynebacterium equi, Corynebacterium pyogenes, and Actinobaccilus seminis.

Non-limiting examples of malaria-specific proteins from which antigenic determinants may be isolated include circumsporozoite (CS) protein, Thrombospondin Related Adhesion (Anonymous) protein (TRAP), also called Sporozoite Surface Protein 2 (SSP2), LSA I, hsp70, SALSA, STARP, Hep17, MSA, RAP-1, RAP-2.

Non-limiting examples of fungal proteins from which antigenic determinants may be isolated include those isolated from candida (e.g., MP65 from Candida albicans), trichophyton, and ptyrosporum.

Non-limiting examples of tumor-associated proteins from which antigenic determinants may be isolated include, e.g., adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1,-A2,-A3,-A4,-A6,-A9,-A10,-A12,-C1, and C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPPIR3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, WT-1. In some embodiments, the peptide is a neo-antigen. In some embodiments, the peptide is a tumor specific antigen.

The term “neo-antigen” or “neo-antigenic” refers to a class of tumor antigens that arises from a tumor-specific mutation(s) which alters one or more amino acids compared to the parental (i.e., genome encoded) protein. For example, a neoantigen may be a tumor-associated neoantigen, wherein the term “tumor-associated neoantigen” includes a peptide or protein including amino acid modifications due to tumor-specific mutations.

In some embodiments, the multispecific molecules described herein are exposed to libraries of synthetically produced peptides to identify the antigenic determinants recognized by a specific T cell. Such peptide libraries include, e.g., peptide libraries produced by PCR (including by introducing random mutations into various positions of a template peptide). A peptide library can include up to 209 or 2×1011 members, or as few as a few hundred to a few thousand members, depending on the knowledge of the peptide binding characteristics of a given MHC. Since 4-5 amino acids are generally involved in MHC binding and cannot directly contact the TCR, prior knowledge of the nature of these amino acids means that only about 5-7 amino acids in the peptide need vary, so that libraries of 106 to 109 members are typically sufficient. In addition, in some embodiments, T cell recognition is dominated by only a few amino acids in the core of the peptide, and in these cases, libraries with only a few hundred to a few thousand members may be sufficient to identify functional peptide-MHC complexes.

Extensive knowledge regarding the binding of peptides to MHC complexes is available to a person of ordinary skill in the art, so that for a given MHC complex, one can design MHC-groove binding peptides that vary in less than all of the available positions. For example, the MHCBN is a comprehensive database of Major Histocompatibility Complex (MHC) binding and non-binding peptides compiled from published literature and existing databases. The latest version of the database has 19,777 entries including 17,129 MHC binders and 2648 MHC non-binders for more than 400 MHCs. The database has sequence and structure data of (a) source proteins of peptides and (b) MHCs. MHCBN has a number of web tools that include: (i) mapping of peptide on query sequence; (ii) search on any field; (iii) creation of data sets; and (iv) online data submission (Bioinformatics 2003 Mar. 22; 19 (5): 665-6).

In one specific embodiment, a library of candidate peptides is produced by genetically engineering the library using polymerase chain reaction (PCR) or any other suitable technique to construct a DNA fragment encoding the peptide. With PCR techniques, by using oligonucleotides that are randomly mutated within particular triplet codons, the resultant fragment pool encodes all possible combination of codons at these positions. Preferably, certain of the amino acid positions are maintained constant, which are the conserved amino acids that are required for binding to the MHC peptide binding groove and which do not contact the T cell receptor. See, e.g., U.S. Pat. Appl. Pub. 2004/0110253.

In this screening method, the target TCR is a TCR for which it is desired to identify the peptide epitope recognized by the receptor. In one embodiment, the target TCR is from a patient with a T cell-mediated disease, such as an autoimmune disease, infection or cancer. See, e.g., Rossjohn and Koning, Mucosal Immunology, 9(3): 583-586 (2016); Qiao et al., J Immunol., 187:3064-3071 (2011); Broughton et al., Immunity, 37:611-621 (2012); Qiao et al., International Immunology, 26 (1): 13-19.

Attaching the peptide to the MHC Class I or MHC Class II molecule via a flexible linker has the advantage of assuring that the peptide will occupy and stay associated with the MHC during biosynthesis, transport and display. However, there may be situations in which this linker interferes with peptide binding to the MHC or with TCR recognition of the complex. As an alternate approach, in some embodiments, the MHC and the peptide are expressed separately. In certain embodiments, the separately expressed peptide is then loaded onto the MHC molecule.

A “single chain peptide-major histocompatibility complex (pMHC) fusion” is a single chain polypeptide comprising a peptide fused to one or more domains of an MHC protein, optionally wherein the peptide and the one or more domains of the MHC protein are joined together by one or more linkers.

The term “fusion”, as used herein, means (but is not limited to) a polypeptide formed by expression of a chimeric gene made by combining more than one sequence, typically by cloning one gene into an expression vector in frame with a second gene such that the two genes are encoding one continuous polypeptide. Recombinant cloning techniques, such as polymerase chain reaction (PCR) and restriction endonuclease cloning, are well-known in the art. In addition to being made by recombinant technology, parts of a polypeptide can be fused to each other to form a “fusion” by means of chemical reaction, or other means known in the art for making custom polypeptides.

T-cell surface molecules (e.g., immunomodulatory molecules) targeted by the antigen-binding domain of the second molecule included within the multispecific molecules described herein are molecules that can anchor the multispecific molecule and/or mediate a T cell response, including, but not limited to, proliferation, activation, differentiation, and the like. The immunomodulatory molecules, specifically T-cell immunomodulatory molecules, can be, for example, CD28, CD80, CD86, CD3, CD4, CD7, CD8, CD27, CD47, CD70, CD83, BTLA, 4-1BB, 4-1BBL, OX40, OX40L, CD30, CD40, CD40L, CD70, CD160, CTLA4, PD1, PD-L1, PDL2, ICOS, ICOSL, galectin 9, GITR, GITRL, ILT3, ILT4, lymphocyte function-associated antigen-1 (LFA-1), LFA-3, LIGHT, MHC-1, NKG2C, TIM1, TIM3, TIM4, Toll ligand receptor, B7-H3, B7-H4, HVEM, CD79a, CD79b, IgSF CAMS (including CD2, CD58, CD48, CD150, CD229, CD244, ICAM-1), Leukocyte immunoglobulin like receptors (LILR), killer cell immunoglobulin like receptors (KIR)), lectin superfamily members, selectins, cytokines/chemokine and cytokine/chemokine receptors, growth factors and growth factor receptors), adhesion molecules (integrins, fibronectins, cadherins), or ecto-domains of multi-span integral membrane proteins. In certain embodiments, the T-cell surface molecule is targeted by the domain that specifically binds a molecule expressed on the surface of the cell expressing the TCR of the second molecules described herein (e.g., CD28, CD27, BTLA, 4-1BB, OX40, CD40L, CD160, CTLA4, PD1, ICOS, galectin 9, GITR, lymphocyte function-associated antigen-1 (LFA-1), MHC-1, TIM1, HVEM, CD2, etc.). In certain embodiments, the T-cell surface molecule is the domain that specifically binds a molecule expressed on the surface of the cell expressing the TCR of the second molecules described herein (e.g., CD80, CD86, CD40, ICOSL, CD70, OX40L, 4-1BBL, GITRL, LIGHT, TIM3, TIM4, ICAM1, LFA3, PDL-1, PD-L2, B7-H3, B7-H4, HVEM, ILT3, ILT4, 2B4, CD226, etc.).

The expression “T-cell immunomodulatory molecule,” as used herein, encompasses “T-cell co-stimulatory molecules” and “T-cell inhibitory molecules.” A “T-cell co-stimulatory molecule,” as used herein, refers to a protein expressed by a T cell that binds a cognate ligand or receptor (e.g., on an antigen-presenting cell) to provide a stimulatory signal, which, in combination with the primary signal provided by engagement of the T cell's TCR with a peptide/MHC, stimulates the activity of the T cell. Stimulation outcome can only be achieved when in combination with the primary TCR signal. Stimulation of a T cell can include activation, proliferation and/or survival of the T cell. Non-limiting examples of T-cell co-stimulatory molecules includes CD28, CD40L, ICOS, CD27, OX40, 4-1BB, GITR, HVEM, galectin 9, LFA-1, DR3, CD30 SLAM. 2B4, CD226, TIM1, TIM2, and CD2.

A “T-cell inhibitory molecule,” as used herein, refers to a protein expressed by a T cell that binds a cognate ligand or receptor (e.g., on an antigen-presenting cell) to provide an inhibitory signal, which, in combination with the primary signal provided by engagement of the T cell's TCR with a peptide/MHC, inhibits the activity of the T cell. Inhibition of a T cell can include anergy, suppression of activity or proliferation and/or death of the T cell. Non-limiting examples of T-cell co-stimulatory molecules includes CTLA4, PD1, BTLA, TIM3, TIGIT, CD160, LAG3, LAIR1, MHC-1, B7-1 and B7-H1.

As used herein, the expression “cell surface-expressed” or “cell-surface molecule” means one or more protein(s) that is/are expressed on the surface of a cell in vitro, ex-vivo, or in vivo, such that at least a portion of the protein is exposed to the extracellular side of the cell membrane and is accessible to an antigen-binding portion of an antibody or an antigen-binding domain of the multispecific molecules discussed herein.

The term “antigen-binding domain,” as used herein, means any antigen-binding molecule or molecular complex comprising at least one complementarity determining region (CDR) that specifically binds to or interacts with a particular antigen (e.g., CD28 or CTLA-4). The term “monovalent antigen-binding domain” or “one-arm antibody” includes immunoglobulin molecules comprising two polypeptide chains (one heavy (H) chain and one light (L) chain) inter-connected by disulfide bonds. The heavy chain comprises a heavy chain variable region (abbreviated herein as HCVR or V_{H}) and a heavy chain constant region. The heavy chain constant region comprises three domains, C_{H}1, C_{H}2 and C_{H}3. The light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the antigen-binding domains may be identical to the human germline sequences, or may be naturally or artificially modified. An amino acid consensus sequence may be defined based on a side-by-side analysis of two or more CDRs.

The terms “antigen-binding domain” and “monovalent antigen-binding domain,” as used herein, also include antigen-binding fragments of the molecules discussed above. The antigen-binding fragments include any naturally occurring, enzymatically obtainable, synthetic, or genetically engineered polypeptide or glycoprotein that specifically binds an antigen to form a complex. Antigen-binding fragments may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc. Non-limiting examples of antigen-binding fragments include: (i) Fab fragments; (ii) Fab′ fragments; (iii) Fd fragments; (iv) Fv fragments; (v) and single-chain Fv (scFv) molecules.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 3, 4, 5, 6, 7, 8, 9 10, 15, 20, 30, 40, 50, 60 or more) amino acids which result in a flexible or semi-flexible linkage between adjacent variable and/or constant domains in a single polypeptide molecule. Moreover, an antigen-binding fragment of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

In certain embodiments of the invention, the antigen-binding domain and/or the MHC portion of the pMHC fusion are human. The term “human antigen-binding domain,” as used herein, is intended to include antigen-binding domains having variable and constant regions derived from human germline immunoglobulin sequences. The human antigen-binding domains of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antigen-binding domain”, as used herein, is not intended to include antigen-binding domains in which CDR sequences derived from the germline of another mammalian species, such as a mouse, have been grafted onto human framework sequences. Similarly, the term “human MHC,” as used herein, is intended to refer to an MHC molecule in which the various domains are encoded by human MHC genes.

In certain embodiments of the invention, the antigen-binding domain of the second molecule included within the multispecific molecules of the invention is a monoclonal antibody, synthetic antibody, recombinantly produced antibody, multispecific antibody, human antibody, chimeric antibody, camelized antibody, single-chain Fvs (scFv), single chain antibody, Fab fragment, F(ab′) fragment, disulfide-linked Fvs (sdFv), intrabody, or epitope-binding fragment of any of the above. In certain embodiments, the antigen-binding domain of the second molecule included within the multispecific molecules of the invention, or antigen-binding fragments thereof, are Ig-DARTS or covalent diabodies such as those disclosed in U.S. Pat. Appl. Pub. 2007/0004909.

The antigen-binding domain of the second molecule included within the multispecific molecules of the invention may be humanized by any method known in the art for modifying proteins for therapeutic use in humans. In addition to methods commonly known in the art for combining heterologous CDR sequences with human framework and/or constant domains, the term “humanization” also includes methods of protein and/or antibody resurfacing such as those disclosed, e.g., in U.S. Pat. Nos. 5,770,196; 5,776,866; 5,821,123; and 5,896,619.

The antigen-binding domain of the second molecule included within the multispecific molecules of the invention may be derived from any species (e.g., rabbit, mouse, rat, donkey, cow, camel, llama, sheep, goat, horse, primate), but is preferably derived from human immunoglobulin molecules that can be of any type (e.g., IgG, IgE, IgM, IgD, IgA and IgY), or class (e.g., IgG1, IgG2, IgG3, IgG4, IgA1, and IgA2) or subclass. The antigen-binding domain of the second molecule included within the multispecific molecules of the invention can be produced by any method known in the art, for example, chemical synthesis or recombinant techniques.

The invention encompasses antigen-binding domains comprising one or more amino acid modifications which, e.g., alter antibody binding or effector functions. See, e.g., U.S. Pat. Appl. Pub. Nos. U.S. 2005/0037000 and U.S. 2005/0064514; U.S. Pat. Nos. 5,624,821 and 5,648,260; European Pat. No. EP0307434; Int. Pat. Appl. Pub. Nos. WO 04/029207, WO 04/029092, WO 04/028564, WO 99/58572, WO 99/51642, WO 98/23289, WO 89/07142, WO 88/07089, U.S. Pat. Nos. 5,843,597 and 5,642,821). In some embodiments, mutation of the amino acids of a protein creates an equivalent, or even an improved, second-generation molecule. For example, certain amino acids may be substituted for other amino acids in a protein structure without detectable loss of affinity or avidity.

The present invention encompasses antigen-binding domains which are fused to or chemically conjugated (including both covalently and non-covalently conjugations) to heterologous polypeptides. In some embodiments, such fusion proteins comprise linker sequences.

Modified antibodies or fragments thereof can be produced, e.g., by random mutagenesis by error-prone PCR, random nucleotide insertion or other methods prior to recombination, DNA shuffling, etc.

For some uses, it may be preferable to use human or chimeric antibodies or fragments thereof. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and Int. Pat. App. Pub. Nos. WO 98/46645, WO 98/50433, WO 98/24893, WO98/16654, WO 96/34096, WO 96/33735, and WO 91/10741.

Single domain antibodies, for example, antibodies lacking the light chains, can be produced by methods well-known in the art. See, e.g., Riechmann et al., 1999, J. Immunol. 231:25-38; Nuttall et al., 2000, Curr. Pharm. Biotechnol. 1(3): 253-263; Muylderman, 2001, J. Biotechnol. 74(4): 277302; U.S. Pat. No. 6,005,079; and Int. Pat. App. Pub. Nos. WO 94/04678, WO 94/25591, and WO 01/44301.

The term “specifically binds,” “binds in a specific manner,” “antigen-specific” or the like, indicates that the molecules involved in the specific binding are able to form a complex with each other that is relatively stable under physiological conditions, and are unable to form stable complexes non-specifically with other molecules outside the specified binding pair. In certain embodiments, the antigen is a TCR, and the pMHC complex acts as a TCR-binding molecule. Accordingly, a pMHC complex that binds in a specific manner to an antigen-specific TCR indicates not only that the pMHC complex forms a stable complex with the antigen specific TCR, but also the antigen-specific TCR forms a stable complex with a distinct antigen. Accordingly, a pMHC complex that binds in a specific manner to an antigen-specific TCR may be considered the antigen to which the TCR is specific, e.g., the pMHC complex (i) does not target the constant domain(s) of a TCR or other components of a TCR complex (e.g., CD3) at all, (ii) targets the constant domain(s) of a TCR or other components of a TCR complex (e.g., CD3) in addition to targeting the variable domain(s) and/or idiotype of a TCR, or (iii) solely targets the variable domain(s) and/or idiotype of a TCR. Specific binding can be characterized by an equilibrium dissociation constant (KD) of about 3000 nM or less (i.e., a smaller KD denotes a tighter binding), about 2000 nM or less, about 1000 nM or less; about 500 nM or less; about 300 nM or less; about 200 nM or less; about 100 nM or less; about 50 nM or less; about 1 nM or less; or about 0.5 nM or less. Methods for determining whether two molecules specifically bind are well known in the art and include, for example, equilibrium dialysis, surface plasmon resonance, and the like.

As used herein, a “multimerization domain” is any macromolecule that has the ability to associate (covalently or non-covalently) with a second macromolecule of the same or similar structure or constitution. For example, a multimerization domain may be a polypeptide comprising an immunoglobulin C_{H}3 domain. A non-limiting example of a multimerization domain is an Fc portion of an immunoglobulin of any type (e.g., IgG, IgE, IgM, IgD, IgA and IgY), class (e.g., IgG1, IgG2, IgG3, IgG4, IgAQ1 and IgA2) or subclass, e.g., an Fc domain of an IgG selected from the isotypes IgG1, IgG2, IgG3, and IgG4, as well as any allotype within each isotype group. In certain embodiments, the multimerization domain is an Fc fragment or an amino acid sequence of 1 to about 200 amino acids in length containing at least one cysteine residue. In other embodiments, the multimerization domain is a cysteine residue or a short cysteine-containing peptide. Other multimerization domains include peptides or polypeptides comprising or consisting of a leucine zipper, a helix-loop motif, or a coiled-coil motif.

As used herein, the terms “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3 oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “recombinant,” as used herein, is intended to include all molecules that are prepared, expressed, created or isolated by recombinant means, such as multispecific molecules (e.g. bispecific molecules) expressed using a recombinant expression vector transfected into a host cell (described further below), multispecific molecules (e.g., bispecific molecules) isolated from an animal (e.g., a mouse) that is transgenic for human immunoglobulin genes (see e.g., Taylor et al. (1992) Nucl. Acids Res. 20:6287-6295) or multispecific molecules prepared, expressed, created or isolated by any other means that involves splicing of human immunoglobulin and/or MHC gene sequences to other DNA sequences. Such recombinant multispecific molecules can include antigen-binding domains having variable and constant regions derived from human germline immunoglobulin sequences.

The term “subject,” “individual,” animal,” or “patient” as used interchangeably herein includes all members of the animal kingdom including non-human primates and humans, veterinary animals (e.g., cats, dogs, cows, horses, sheep, pigs, etc.) and experimental animal models of diseases (e.g., mice, rats). In a preferred embodiment, the subject is a human. In one embodiment, patients are humans with a disease or disorder, e.g., an infection, a cancer or an autoimmune disorder.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The terms “protein,” “polypeptide,” and “peptide,” encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains:glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

The terms “vector” and “expression vector,” as used herein, include, but are not limited to, a viral vector, a plasmid, an RNA vector or a linear or circular DNA or RNA molecule which may consist of chromosomal, non-chromosomal, semi-synthetic or synthetic nucleic acids. In some embodiments, the vectors are those capable of autonomous replication (episomal vector) and/or expression of nucleic acids to which they are linked (expression vectors). Large numbers of suitable vectors are known to those of skill in the art and are commercially available. Viral vectors include retrovirus, adenovirus, parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, and lentivirus.

In accordance with the disclosure herein, there may be employed conventional molecular biology, microbiology, and recombinant DNA techniques within the skill of the art.

Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82:488-492 (1985), U.S. Pat. No. 5,071,743, Fukuoka et al., Biochem. Biophys. Res. Commun. 263:357-360 (1999); Kim and Maas, BioTech. 28:196-198 (2000); Parikh and Guengerich, BioTech. 24:4 28-431 (1998); Ray and Nickoloff, BioTech. 13:342-346 (1992); Wang et al., BioTech. 19:556-559 (1995); Wang and Malcolm, BioTech. 26:680-682 (1999); Xu and Gong, BioTech. 26:639-641 (1999), U.S. Pat. Nos. 5,789,166 and 5,932,419, Hogrefe, Strategies 14. 3:74-75 (2001), U.S. Pat. Nos. 5,702,931, 5,780,270, and 6,242,222, Angag and Schutz, Biotech. 30:486-488 (2001), Wang and Wilkinson, Biotech. 29:976-978 (2000), Kang et al., Biotech. 20:44-46 (1996), Ogel and McPherson, Protein Engineer. 5:467-468 (1992), Kirsch and Joly, Nucl. Acids. Res. 26:1848-1850 (1998), Rhem and Hancock, J. Bacteriol. 178:3346-3349 (1996), Boles and Miogsa, Curr. Genet. 28:197-198 (1995), Barrenttino et al., Nuc. Acids. Res. 22:541-542 (1993), Tessier and Thomas, Meths. Molec. Biol. 57:229-237, and Pons et al., Meth. Molec. Biol. 67:209-218.

### Multispecific Molecules and First and Second Molecules of the Multispecific Carriers

The multispecific molecules of the present invention are composed of two heterologous binding molecules for engaging a T cell and either suppressing or inducing an immune response. In certain embodiments, the multispecific molecule comprises: (i) a peptide (p) presented in the context of a major histocompatibility complex (MHC) molecule (pMHC complex), and (ii) a first multimerization domain; and a second molecule that is (i) a domain that specifically binds a molecule expressed on the surface of the cell expressing the TCR, and (ii) a second multimerization domain. The multispecific molecules of the present invention do not contain an MHC protein or MHC domain on both binding molecules.

The multispecific carrier molecules of the present inventions express on their surfaces two heterologous transmembrane molecules for engaging a T cell and either suppressing or inducing an immune response. In certain embodiments, the multispecific carrier comprises a plurality of first molecules and a plurality of second molecules, wherein each first molecule a peptide (p) presented in the context of a major histocompatibility complex (MHC) molecule (pMHC complex), and
each second molecule is a polypeptide comprising a domain that specifically binds a molecule expressed on the surface of the cell expressing the TCR.

### First Molecule of the Multispecific Molecules or Multispecific Carriers

The first molecule of the multispecific molecules or multispecific carriers can include one or more domains of a class I or a class II MHC protein, or fragments, mutants or derivatives thereof, fused to a peptide that can be recognized by the TCR of a T cell. In some embodiments, e.g., where the target T cell is a CD8+ T cell, the multispecific molecule or multispecific carrier described herein comprises a class I MHC polypeptide or fragment, mutant or derivative thereof. In some embodiments, e.g., where the target T cell is a CD4+ T cell, the multispecific molecule or multispecific carrier described herein comprises class II MHC polypeptides or fragment, mutant or derivative thereof.

In one embodiment, the first molecule includes one or more domains of a class I MHC protein or fragment, mutant or derivative thereof. For example, the first molecule can include one or more of the alpha 1, alpha 2, and alpha 3 domains and beta 2 microglobulin domain of a class I MHC protein. In one embodiment, the first molecule includes all of the alpha 1, 2 and 3 chain domains, and the beta 2 microglobulin domain.

In one embodiment of the multispecific molecule, the first molecule comprises, from N-terminus to C-terminus, (i) a peptide, (ii) a beta 2 microglobulin peptide or fragment, mutant or derivative thereof, (iii) a class I alpha domain or fragment, mutant or derivative thereof, and (iv) a multimerization domain (e.g., an immunoglobulin Fc domain). In one embodiment of the multispecific molecule, the first molecule comprises, from N-terminus to C-terminus, (i) a peptide, (ii) a beta 2 microglobulin peptide or fragment, mutant or derivative thereof, (iii) an alpha 1-class I domain peptide or fragment, mutant or derivative thereof, (iv) an alpha 2-class I domain peptide or fragment, mutant or derivative thereof, (v) an alpha 3 domain peptide or fragment, mutant or derivative thereof, and (vi) a multimerization domain (e.g., an immunoglobulin Fc domain). In one embodiment of the multispecific molecule, the first molecule comprises, from N-terminus to C-terminus, (i) a peptide, (ii) an alpha 3 domain peptide or fragment, mutant or derivative thereof, (iii) an alpha 2-class I domain peptide or fragment, mutant or derivative thereof, (iv) an alpha 1-class I domain peptide or fragment, mutant or derivative thereof, (v) a beta 2 microglobulin peptide or fragment, mutant or derivative thereof, and (vi) a multimerization domain (e.g., an immunoglobulin Fc domain). In some embodiments, the peptide, class I MHC domains and/or the multimerization domain are joined by one or more peptide linkers.

In one embodiment of the multispecific carrier, the first molecule comprises, from N-terminus to C-terminus, (i) a peptide, (ii) a beta 2 microglobulin peptide or fragment, mutant or derivative thereof, (iii) a class I alpha domain or fragment, mutant or derivative thereof, and (iv) a transmembrane domain. In one embodiment of the multispecific carrier, the first molecule of the multispecific carrier comprises, from N-terminus to C-terminus, (i) a peptide, (ii) a beta 2 microglobulin peptide or fragment, mutant or derivative thereof, (iii) an alpha 1-class I domain peptide or fragment, mutant or derivative thereof, (iv) an alpha 2-class I domain peptide or fragment, mutant or derivative thereof, (v) an alpha 3 domain peptide or fragment, mutant or derivative thereof, and (vi) a transmembrane domain. In one embodiment of the multispecific carrier, the first molecule comprises, from N-terminus to C-terminus, (i) a peptide, (ii) an alpha 3 domain peptide or fragment, mutant or derivative thereof, (iii) an alpha 2-class I domain peptide or fragment, mutant or derivative thereof, (iv) an alpha 1-class I domain peptide or fragment, mutant or derivative thereof, (v) a beta 2 microglobulin peptide or fragment, mutant or derivative thereof, and (vi) a transmembrane domain. In some embodiments, the peptide, class I MHC domains and/or the transmembrane domain are joined by one or more peptide linkers.

In some embodiments, the class I MHC polypeptide is a human class I MHC polypeptide such as, but not limited to, HLA-A, HLA-B, HLA-C, HLA-E, HLA-F, and HLA-G. In other embodiments, the class I MHC polypeptide is a murine class I MHC polypeptide such as, but not limited to, H-2K, H-2D, H-2L, H2-IA, H2-IB, H2-IJ, H2-IE, and H2-IC.

In some embodiments, the MHC class I alpha heavy chain is fully human. In some embodiments, the MHC class I alpha heavy chain is humanized. Humanized MHC class I alpha heavy chains are described, e.g., in U.S. Pat. Pub. Nos. 2013/0111617, 2013/0185819 and 2014/0245467. In some embodiments, the MHC class I alpha heavy chain comprises a human extracellular domain (human alpha1, alpha2, and/or alpha3 domains) and a cytoplasmic domain of another species. In some embodiments, the class I alpha heavy chain polypeptide is HLA-A, HLA-B, HLA-C, HLA-E, HLA-F, HLA-G, HLA-K, or HLA-L. In some embodiments, the HLA-A sequence can be an HLA-A*0201 sequence. In various aspects, the peptide-MHC can include all the domains of an MHC class I heavy chain.

In some embodiments, the β2-microglobulin is fully human. In some embodiments, the β2-microglobulin is humanized. Humanized β2-microglobulin polypeptides are described, e.g., in U.S. Pat. Pub. Nos. 2013/0111617 and 2013/0185819.

In some embodiments, the MHC class I molecule comprises a mutation in a β2-microglobulin (β2m or B2M) polypeptide and in the Heavy Chain sequence so as to affect a disulfide bond between the B2M and the Heavy Chain. In some embodiments, the Heavy Chain is an HLA and wherein the disulfide bond links one of the following pairs of residues: B2M residue 12, HLA residue 236; B2M residue 12, HLA residue 237; B2M residue 8, HLA residue 234; B2M residue 10, HLA residue 235; B2M residue 24, HLA residue 236; B2M residue 28, HLA residue 232; B2M residue 98, HLA residue 192; B2M residue 99, HLA residue 234; B2M residue 3, HLA residue 120; B2M residue 31, HLA residue 96; B2M residue 53, HLA residue 35; B2M residue 60, HLA residue 96; B2M residue 60, HLA residue 122; B2M residue 63, HLA residue 27; B2M residue Arg3, HLA residue Glyl20; B2M residue His31, HLA residue Gln96; B2M residue Asp53, HLA residue Arg35; B2M residue Trp60, HLA residue Gln96; B2M residue Trp60, HLA residue Asp122; B2M residue Tyr63, HLA residue Tyr27; B2M residue Lys6, HLA residue Glu232; B2M residue Gln8, HLA residue Arg234; B2M residue TyrIO, HLA residue Pro235; B2M residue Serl 1, HLA residue Gln242; B2M residue Asn24, HLA residue Ala236; B2M residue Ser28, HLA residue Glu232; B2M residue Asp98, HLA residue His 192; and B2M residue Met99, HLA residue Arg234, first linker position Gly 2, Heavy Chain (HLA) position Tyr 84; Light Chain (B2M) position Arg 12, HLA Ala236; and/or B2M residue Arg12, HLA residue Gly237. See, e.g., Int. Pat. Appl. Pub. WO2015/195531, incorporated by reference herein for all intended purposes.

In certain embodiments, the pMHC complex can comprise a peptide covalently attached to an MHC class I a (heavy) chain via a disulfide bridge (i.e., a disulfide bond between two cystines). In certain embodiments, the disulfide bond comprises a first cysteine, comprised by a linker extending from the carboxy terminal of an antigen peptide, and a second cysteine comprised by an MHC class I heavy chain (e.g., an MHC class I a (heavy) chain which has a non-covalent binding site for the antigen peptide). In certain embodiments, the second cysteine can be a mutation (addition or substitution) in the MHC class I a (heavy) chain. In certain embodiments, the pMHC complex can comprise one contiguous polypeptide chain as well as a disulfide bridge. In certain embodiments, the pMHC complex can comprise two contiguous polypeptide chains which are attached via the disulfide bridge as the only covalent linkage. In some embodiments, the linking sequences can comprise at least one amino acid in addition to the cysteine, including one or more glycines, one or more, alanines, and/or one or more serines.

In certain embodiments, the disulfide bridge can link an antigen peptide in the class I groove of the pMHC complex if the pMHC complex comprises a first cysteine in a Gly-Ser linker extending between the C-terminus of the peptide and the β2-microglobulin, and a second cysteine in a proximal heavy chain position.

In some embodiments, the β2-microglobulin sequence can comprise a full-length β2-microglobulin sequence. In certain embodiments, the β2-microglobulin sequence lacks the leader peptide sequence. As such, in some configurations, the β2-microglobulin sequence can comprise about 99 amino acids, and can be a mouse β2-microglobulin sequence (e.g., Genebank X01838). In some other configurations, the β2-microglobulin sequence can comprise about 99 amino acids, and can be a human β2-microglobulin sequence (e.g., Genebank AF072097.1).

In some embodiments, the pMHC complex sequence can be that as disclosed in U.S. Pat. Nos. 4,478,82; 6,011,146; 8,518,697; 8,895,020; 8,992,937; WO 96/04314; Mottez et al. J. Exp. Med. 181:493-502, 1995; Madden et al. Cell 70:1035-1048, 1992; Matsumura et al., Science 257:927-934, 1992; Mage et al., Proc. Natl. Acad. Sci. USA 89:10658-10662, 1992; Toshitani et al, Proc. Nat'l Acad. Sci. 93:236-240, 1996; Chung et al, J. Immunol. 163:3699-3708, 1999; Uger and Barber, J. Immunol. 160:1598-1605, 1998; Uger et al., J. Immunol. 162, pp. 6024-6028, 1999; White et al., J. Immunol. 162:2671-2676, 1999; Yu et al., J. Immunol. 168:3145-3149, 2002; Truscott et al., J. Immunol. 178:6280-6289, 2007.

In certain embodiments, the first binding molecule includes one or more domains of a class II MHC protein. For example, the first binding molecule can include one or more of the alpha 1, alpha 2, beta 1 and beta 2 domains of a class II MHC protein. In one embodiment, the first binding molecule includes all of the alpha 1, alpha 2, beta 1 and beta 2 domains.

In certain embodiments of the multispecific molecule, the first binding molecule comprises, from N-terminus to C-terminus, (i) a peptide, (ii) a class II alpha domain or fragment, mutant or derivative thereof, (iii) a class II beta domain or fragment, mutant or derivative thereof, and (iv) a multimerization domain (e.g., an immunoglobulin Fc domain). In one embodiment of the multispecific molecule, the first binding molecule comprises, from N-terminus to C-terminus, (i) a peptide, (ii) a class II beta domain or fragment, mutant or derivative thereof, (iii) a class II alpha domain or fragment, mutant or derivative thereof, and (iv) a multimerization domain (e.g., an immunoglobulin Fc domain). In one embodiment of the multispecific molecule, the first binding molecule comprises, from N-terminus to C-terminus, (i) a peptide, (ii) an alpha 2-class II domain peptide or fragment, mutant or derivative thereof, an (iii) alpha 1-class II domain peptide or fragment, mutant or derivative thereof, (iv) a beta 1 domain peptide or fragment, mutant or derivative thereof, (v) a beta 2 domain peptide or fragment, mutant or derivative thereof, and (vi) a multimerization domain (e.g., an immunoglobulin Fc domain). In one embodiment of the multispecific molecule, the first binding molecule comprises, from N-terminus to C-terminus, (i) a peptide, (ii) a beta 2 domain peptide or fragment, mutant or derivative thereof, (iii) a beta 1 domain peptide or fragment, mutant or derivative thereof, (iv) an alpha 1-class II domain peptide or fragment, mutant or derivative thereof, (v) an alpha 2-class II domain peptide or fragment, mutant or derivative thereof, and (vi) a multimerization domain (e.g., an immunoglobulin Fc domain). In some embodiments, the peptide, class II MHC domains and/or the multimerization domain are joined by one or more peptide linkers.

In certain embodiments of the multispecific carrier, the first binding molecule comprises, from N-terminus to C-terminus, (i) a peptide, (ii) a class II alpha domain or fragment, mutant or derivative thereof, (iii) a class II beta domain or fragment, mutant or derivative thereof, and (iv) a transmembrane domain. In one embodiment of the multispecific carrier, the first binding molecule comprises, from N-terminus to C-terminus, (i) a peptide, (ii) a class II beta domain or fragment, mutant or derivative thereof, (iii) a class II alpha domain or fragment, mutant or derivative thereof, and (iv) a transmembrane domain. In one embodiment of the multispecific carrier, the first binding molecule comprises, from N-terminus to C-terminus, (i) a peptide, (ii) an alpha 2-class II domain peptide or fragment, mutant or derivative thereof, an (iii) alpha 1-class II domain peptide or fragment, mutant or derivative thereof, (iv) a beta 1 domain peptide or fragment, mutant or derivative thereof, (v) a beta 2 domain peptide or fragment, mutant or derivative thereof, and (vi) a transmembrane domain. In one embodiment of the multispecific carrier, the first binding molecule comprises, from N-terminus to C-terminus, (i) a peptide, (ii) a beta 2 domain peptide or fragment, mutant or derivative thereof, (iii) a beta 1 domain peptide or fragment, mutant or derivative thereof, (iv) an alpha 1-class II domain peptide or fragment, mutant or derivative thereof, (v) an alpha 2-class II domain peptide or fragment, mutant or derivative thereof, and (vi) a transmembrane domain. In some embodiments, the peptide, class II MHC domains and/or the transmembrane domain are joined by one or more peptide linkers.

In some embodiments, the MHC comprises α and β polypeptides (or fragments thereof, e.g., the α1 and β1 domains) of a human class II MHC complex selected from the group consisting of HLA DP, HLA-DR, HLA-DQ, HLA-DM and HLA-DO. In another specific embodiment, the MHC comprises α and β polypeptides (or fragments thereof, e.g., the α1 and β1 domains) of a murine H-2A or H-2E class II MHC complex.

Naturally occurring MHC class II molecules consist of two polypeptide chains, a and B. The chains may come from the DP, DQ, or DR gene groups. There are about 40 known different human MHC class II molecules. All have the same basic structure, but vary subtly in their molecular structure. MHC class II molecules bind peptides of 13-18 amino acids in length.

In some embodiments, the multispecific molecule comprises one or more MHC class II α chains. In some embodiments, the MHC class II α chain is fully human. In some embodiments, the MHC class II α chain is humanized. Humanized MHC class II α chains are described, e.g., in U.S. Pat. Nos. 8,847,005 and 9,043,996 and U.S. Pat. Pub. No. 2014/0245467. In some embodiments, the humanized MHC class II α chain polypeptide comprises a human extracellular domain and a cytoplasmic domain of another species. In some embodiments, the class II α chain is HLA-DMA, HLA-DOA, HLA-DPA, HLA-DQA or HLA-DRA. In some embodiments, the class II α chain polypeptide is humanized HLA-DMA, HLA-DOA, HLA-DPA, HLA-DQA and/or HLA-DRA.

In some embodiments, the multispecific molecule comprises one or more MHC class II β chains. In some embodiments, the MHC class II β chain is fully human. In some embodiments, the MHC class II β chain polypeptide is humanized. Humanized MHC class II β chain polypeptides are described, e.g., in U.S. Pat. Nos. 8,847,005 and 9,043,996 and U.S. Pat. Pub. No. 2014/0245467. In some embodiments, the humanized MHC class II β chain comprises a human extracellular domain and a cytoplasmic domain of another species. In some embodiments, the class II β chain is HLA-DMB, HLA-DOB, HLA-DPB, HLA-DQB or HLA-DRB. In some embodiments, the class II β chain is humanized HLA-DMB, HLA-DOB, HLA-DPB, HLA-DQB and/or HLA-DRB.

The first binding molecule of the multispecific molecules of the invention is structured such that the peptide can be positioned in the groove formed by, e.g., the class I alpha 1 and alpha 2 domain peptides or the class II alpha 1 and beta 1 domain peptides for presentation to a T cell's TCR. In various embodiments, the peptide can consist of from about 5 to about 40 amino acid residues, from about 6 to about 30 amino acid residues, from about 7 to about 25 amino acid residues, or from about 8 to about 20 amino acid residues. In some embodiments, the peptide can consist of from about 5 to about 15 amino acid residues, from about 8 to about 12 amino acid residues, or about 8, about 9, about 10, about 11, or about 12 amino acid residues.

In some embodiments, the peptide is derived from a viral antigen. In some embodiments, the viral antigen is a viral protein or fragment of a viral protein associated with a virus selected from the group consisting of adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, and zika.

In some embodiments, the peptide is derived from a bacterial antigen. In some embodiments, the bacterial antigen is an antigen associated with a bacterium that is resistant to conventional antibiotic treatments, such as methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, or Clindamycin-resistant group B Streptococcus.

In some embodiments, the peptide is derived from a tumor-associated antigen. The term “tumor-associated antigen,” as used herein, refers to a protein expressed by, or over-expressed (in comparison to non-tumor cells) by, tumor cells. In some embodiments, the tumor-associated antigen is selected from the group consisting of adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, WT-1. In some embodiments, the peptide is a neo-antigen. In some embodiments, the peptide is a tumor specific antigen.

In some embodiments, the peptide is derived from an antigen associated with an autoimmune disorder. In some embodiments, the antigen associated with an autoimmune disorder is selected from the group consisting of gliadin (celiac disease; e.g., (i) α-gliadin fragment corresponding to amino acids 57-73 or (ii) γ-gliadin fragment corresponding to amino acids 139 153 or (iii) ω-gliadin fragment corresponding to amino acids 102-118), GAD 65, IA-2 and insulin β chain (for type 1-diabetes), glatiramer acetate (GA) (for multiple sclerosis), acetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)). In some embodiments, the antigen associated with an autoimmune disorder is selected from the group consisting of IL-4R (interleukin-4 receptor), IL-6R (interleukin-6 receptor) and DLL4 (delta-like ligand 4).

In some embodiments, the components or peptides of the first binding molecule are separated by a linker (or “spacer”) peptide. Such peptide linkers are well known in the art (e.g., polyglycine) and typically allow for proper folding of one or both of the components of the fusion polypeptide. The linker provides a flexible junction region of the component of the fusion polypeptide, allowing the components of the molecule to move independently. Therefore, the junction region acts in some embodiments as both a linker, which combines the two parts together, and as a spacer, which allows each of the connected parts to form its own biological structure and not interfere with the other part. Furthermore, the junction region should create an epitope that will not be recognized by the subject's immune system as foreign, in other words, will not be considered immunogenic. In one embodiment, each of the respective class I or class II MHC domains and the peptide are connected to one another via peptide linkers.

Suitable linkers used in the MHCs can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 2, 3, 4, 5, 6, 7, 8, 9, 10, 11, 12, 13, 14 15 or more amino acid residues, but typically is between 5 and 25 residues. Examples of linkers include polyGlycine linkers, such as Gly-Gly (2Gly), Gly-Gly-Gly (3Gly), 4Gly (SEQ ID NO: 30), 5Gly (SEQ ID NO: 31), 6Gly (SEQ ID NO: 32), 7Gly (SEQ ID NO: 33), 8Gly (SEQ ID NO: 34) or 9Gly (SEQ ID NO: 35). Examples of linkers also include Gly-Ser peptide linkers such as Ser-Gly (SG), Gly-Ser (GS), Gly-Gly-Ser (G2S), Ser-Gly-Gly (SG2), G3S (SEQ ID NO: 36), SG3 (SEQ ID NO: 37), G4S (SEQ ID NO: 38), SG4 (SEQ ID NO: 39), G5S (SEQ ID NO: 40), SG5 (SEQ ID NO: 41), G6S (SEQ ID NO: 42), SG6 (SEQ ID NO: 43), (G4S) n (SEQ ID NO: 44), (S4G) n (SEQ ID NO: 45), wherein n=1 to 10. Any one of the linkers described herein may be repeated to lengthen the linker as needed. Other flexible linkers known in the art are disclosed in e.g., Chichili et al, Protein Science, 22:153-167 (2013), incorporated herein by reference in its entirety for all purposes. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 1 1173-142 (1992)). Exemplary linkers can comprise amino acid sequences including, but not limited to, GGSG (SEQ ID NO: 46), GGSGG (SEQ ID NO: 47), GSGSG (SEQ ID NO: 48), GSGGG (SEQ ID NO: 49), GGGSG (SEQ ID NO: 50), GSSSG (SEQ ID NO: 51), GCGASGGGGSGGGGS (SEQ ID NO: 52), GCGASGGGGSGGGGS (SEQ ID NO: 52), GGGGSGGGGS (SEQ ID NO: 53), GGGASGGGGSGGGGS (SEQ ID NO: 54), GGGGSGGGGSGGGGS (SEQ ID NO: 55), GGGASGGGGS (SEQ ID NO: 56), GGGGSGGGGSGGGGS (SEQ ID NO: 55) or GGGGGGGGSGGGGSGGGGS (SEQ ID NO: 57) (TABLE 1), and the like. In some embodiments, a linker polypeptide includes a cysteine residue that can form a disulfide bond with a cysteine residue present in a second polypeptide.

TABLE 1

| Linker | | Linker codon- | | Linker codon- | |
|---|---|---|---|---|---|
| amino acids | SEQ ID | optimized nucleotidic | SEQ ID | optimized nucleotidic | SEQ ID |
| sequence | NO: | sequence (human) | NO: | sequence (mouse) | NO: |
| GGGS | 36 | GGGGGTGGTTCC | 59 | GGTGGCGGTAGT | 75 |
| GGSG | 46 | GGTGGGTCTGGG | 60 | GGGGGATCTGGT | 76 |
| GSGGS | 58 | GGGTCCGGGGGCT | 61 | GGCAGTGGCGGTA | 77 |
| | | CC | | GC | |
| GGSGG | 47 | GGTGGGAGCGGTG | 62 | GGAGGGAGTGGA | 78 |
| | | GT | | GGG | |
| GSGSG | 48 | GGCAGCGGAAGCG | 63 | GGGTCTGGCTCAG | 79 |
| | | GA | | GC | |
| GSGGG | 49 | GGGAGTGGGGGA | 64 | GGTTCTGGCGGAG | 80 |
| | | GGT | | GT | |
| GGGSG | 50 | GGTGGGGGAAGTG | 65 | GGTGGTGGGAGTG | 81 |
| | | GA | | GA | |
| GSSSG | 51 | GGCAGCTCATCTG | 66 | GGCTCAAGCAGTG | 82 |
| | | GT | | GA | |
| GCGASGG | 52 | GGATGTGGTGCAT | 67 | GGCTGTGGGGCTA | 83 |
| GGSGGGG | | CTGGAGGGGGAGG | | GTGGGGGAGGTGG | |
| S | | CTCTGGGGGGGGT | | TAGTGGTGGTGGC | |
| | | GGATCT | | GGTTCC | |
| GCGASGG | 52 | GGGTGTGGTGCTA | 68 | GGATGTGGGGCCT | 84 |
| GGSGGGG | | GTGGGGGTGGCGG | | CAGGTGGGGGTGG | |
| S | | ATCAGGTGGAGGC | | CAGCGGTGGTGGA | |
| | | GGGAGC | | GGGTCA | |
| GGGGSGG | 53 | GGGGGCGGAGGAT | 69 | GGTGGCGGGGGCT | 85 |
| GGS | | CTGGGGGAGGGGG | | CTGGTGGAGGAGG | |
| | | ATCA | | ATCT | |
| GGGASGG | 54 | GGGGGGGGCGCTT | 70 | GGAGGCGGCGCTT | 86 |
| GGSGGGG | | CAGGCGGAGGTGG | | CTGGGGGCGGGGG | |
| S | | AAGTGGTGGAGGA | | TAGTGGGGGTGGA | |
| | | GGT | | GGT | |
| GGGGSGG | 55 | GGAGGGGGAGGTT | 71 | GGTGGAGGTGGAA | 87 |
| GGSGGGG | | CTGGCGGCGGGGG | | GTGGAGGAGGGG | |
| S | | ATCAGGAGGCGGT | | GATCAGGCGGAGG | |
| | | GGGAGC | | CGGGAGC | |
| GGGASGG | 56 | GGTGGGGGGGCGT | 72 | GGAGGGGGAGCCT | 88 |
| GGS | | CAGGTGGAGGCGG | | CTGGCGGTGGAGG | |
| | | AAGT | | ATCA | |
| GGGGSGG | 55 | GGCGGCGGAGGTT | 73 | GGGGGAGGAGGC | 89 |
| GGSGGGG | | CTGGTGGGGGTGG | | AGTGGAGGTGGGG | |
| S | | CAGTGGAGGAGGA | | GAAGTGGTGGAGG | |
| | | GGCAGC | | GGGGTCT | |
| GGGGSGG | 57 | GGAGGTGGAGGTA | 74 | GGGGGTGGAGGAT | 90 |
| GGSGGGG | | GTGGCGGTGGTGG | | CAGGAGGCGGTGG | |
| SGGGGS | | GTCAGGGGGAGGC | | TTCTGGGGGAGGT | |
| | | GGGTCCGGTGGCG | | GGATCCGGCGGGG | |
| | | GTGGGAGT | | GTGGTAGT | |

### Second Molecule of the Multispecific Molecules or Multispecific Carriers

The second binding molecule of the multispecific molecules or multispecific carriers can include an antigen-binding domain (e.g., an antibody, one-arm antibody, or antigen-binding fragment thereof) that specifically binds a T-cell surface molecule to anchor the multispecific molecule and/or provide a stimulatory or inhibitory signal to the T cell. In various embodiments, the antigen-binding domain is a protein, polypeptide or molecular complex comprising or consisting of at least one complementarity determining region (CDR) that alone, or in combination with one or more additional CDRs and/or framework regions (FRs), specifically binds to a particular immunomodulatory molecule. In certain embodiments, the antigen-binding domain is part of a one-arm antibody or a fragment thereof, as those terms are defined elsewhere herein.

In certain exemplary embodiments of the present invention, the antigen-binding domain comprises at least one heavy chain and at least one light chain. The heavy chain can include a heavy chain variable region (HCVR) and a heavy chain constant region. The heavy chain constant region can include one or more of CH1, CH2 and/or CH3 domains. The light chain can include a light chain variable region (LCVR) and a light chain constant region. The variable regions of the heavy chain and light chain may contain CDRs designated HCDR1, HCDR2, and HCDR3, and LCDR1, LCDR2, and LCDR3, respectively.

The antigen-binding domain of the second molecule of the multispecific molecules or multispecific carriers specifically binds an immunomodulatory molecule expressed by a T cell. Binding of the immunomodulatory molecule induces or suppresses activation of the T cell in conjunction with the primary signal provided by the binding of the T cell's TCR to a peptide/MHC complex in which the TCR specifically binds the peptide. In some embodiments, the immunomodulatory molecule is a co-stimulatory molecule that induces activation, proliferation and/or survival of the T cell in conjunction with the signal provided by the binding of the T cell's TCR to a peptide/MHC complex. For example, the antigen-binding domain may be a one-armed or two-armed antibody that specifically binds CD28, ICOS, HVEM, CD27, 4-1BB, 0X40, DR3, GITR, CD30, SLAM, CD2, 2B4, CD226, TIM1, or TIM2. In one embodiment, the T cell is a CD8+ T cell. In one embodiment, the T cell is a CD4+ T cell. In some embodiments, the T cell is a CD8+ T cell, and the immunomodulatory molecule bound by the antigen-binding domain is a co-stimulatory molecule such as, but not limited to, CD28, ICOS, HVEM, CD27, 4-1BB, 0X40, DR3, GITR, CD30, SLAM, CD2, 2B4, TIM1, TIM2, and CD226. In some embodiments, the T cell is a CD4+ T cell, and the immunomodulatory molecule bound by the antigen-binding domain is a co-stimulatory molecule such as, but not limited to, CD28, ICOS, HVEM, CD27, 4-1BB, 0X40, DR3, GITR, CD30, SLAM, CD2, TIM1 and TIM2. In other cases, the immunomodulatory molecule is a inhibitory molecule that suppresses activation of the T cell or induces anergy or T cell death in conjunction with the signal provided by the binding of the T cell's TCR to a peptide/MHC complex. For example, the antigen-binding domain may be a one-armed or two-armed antibody that specifically binds CTLA4, PD1, BTLA, TIM3, TIGIT, CD160, LAG3, LAIR1, B7-1 or B7-H1. In one embodiment, the T cell is a CD8+ T cell. In one embodiment, the T cell is a CD4+ T cell. In some embodiments, the T cell is a CD8+ T cell, and the immunomodulatory molecule bound by the antigen-binding domain is a inhibitory molecule such as, but not limited to, CTLA4, PD1, BTLA, TIM3, CD160, LAG3, LAIR1, B7-1 and B7-H1. In some embodiments, the T cell is a CD4+ T cell, and the immunomodulatory molecule bound by the antigen-binding domain is a inhibitory molecule such as, but not limited to, CTLA4, PD1, BTLA, TIM3, TIGIT, CD160, LAG3, LAIR1, B7-1 and B7-H1.

In certain embodiments, the second binding molecule of the multispecific molecules or multispecific carriers can include a small molecule, protein, fusion protein, peptide, aptamer, avimer or derivatives or fragments thereof, with affinity for a molecule expressed on the surface of the cell expressing the TCR. In certain embodiments, the small molecule, protein, fusion protein, peptide, aptamer, avimer or derivatives or fragments thereof, specifically binds an immunomodulatory molecule expressed by a T cell. Binding of the immunomodulatory molecule induces or suppresses activation of the T cell in conjunction with the primary signal provided by the binding of the T cell's TCR to a peptide/MHC complex in which the TCR specifically binds the peptide. In some embodiments, the immunomodulatory molecule is a co-stimulatory molecule that induces activation, proliferation and/or survival of the T cell in conjunction with the signal provided by the binding of the T cell's TCR to a peptide/MHC complex. For example, the small molecule, protein, fusion protein, peptide, aptamer, avimer or derivatives or fragments thereof, may specifically binds CD28, ICOS, HVEM, CD27, 4-1BB, 0X40, DR3, GITR, CD30, SLAM, CD2, 2B4, CD226, TIM1, or TIM2. In one embodiment, the T cell is a CD8+ T cell. In one embodiment, the T cell is a CD4+ T cell. In some embodiments, the T cell is a CD8+ T cell, and the immunomodulatory molecule bound by the small molecule, protein, fusion protein, peptide, aptamer, avimer or derivatives or fragments thereof, is a co-stimulatory molecule such as, but not limited to, CD28, ICOS, HVEM, CD27, 4-1BB, 0X40, DR3, GITR, CD30, SLAM, CD2, 2B4, TIM1, TIM2, and CD226. In some embodiments, the T cell is a CD4+ T cell, and the immunomodulatory molecule bound by the small molecule, protein, fusion protein, peptide, aptamer, avimer or derivatives or fragments thereof, is a co-stimulatory molecule such as, but not limited to, CD28, ICOS, HVEM, CD27, 4-1BB, 0X40, DR3, GITR, CD30, SLAM, CD2, 2B4, CD226, TIM1 and TIM2. In other cases, the immunomodulatory molecule is a inhibitory molecule that suppresses activation of the T cell or induces anergy or T cell death in conjunction with the signal provided by the binding of the T cell's TCR to a peptide/MHC complex. For example, the small molecule, protein, fusion protein, peptide, aptamer, avimer or derivatives or fragments thereof, may specifically binds CTLA4, PD1, BTLA, TIM3, TIGIT, CD160, LAG3, LAIR1, B7-1 or B7-H1. In one embodiment, the T cell is a CD8+ T cell. In one embodiment, the T cell is a CD4+ T cell. In some embodiments, the T cell is a CD8+ T cell, and the immunomodulatory molecule bound by the small molecule, protein, fusion protein, peptide, aptamer, avimer or derivatives or fragments thereof, is a inhibitory molecule such as, but not limited to, CTLA4, PD1, BTLA, TIM3, CD160, LAG3, LAIR1, B7-1 and B7-H1. In some embodiments, the T cell is a CD4+ T cell, and the immunomodulatory molecule bound by the small molecule, protein, fusion protein, peptide, aptamer, avimer or derivatives or fragments thereof, is a inhibitory molecule such as, but not limited to, CTLA4, PD1, BTLA, TIM3, TIGIT, CD160, LAG3, LAIR1, B7-1 and B7-H1.

### Multimerization of the First and Second Molecule

The first binding molecule and the second binding molecule may be directly or indirectly connected to one another to form a multispecific molecule of the present invention. In one embodiment, the first binding molecule and the second binding molecule may each be connected to a separate multimerization domain. The association of one multimerization domain with another multimerization domain facilitates the association between the two binding molecules, thereby forming a multispecific molecule in accordance with the present invention. As used herein, a “multimerization domain” is any macromolecule, protein, polypeptide, peptide, or amino acid that has the ability to associate with a second multimerization domain of the same or similar structure or constitution. For example, a multimerization domain may be a polypeptide comprising an immunoglobulin C_{H}3 domain. A non-limiting example of a multimerization domain is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group. In one embodiment, the multimerization domain is human IgG1. In one embodiment, the multimerization domain is human IgG4.

Multispecific antigen-binding molecules of the present invention will typically comprise two multimerization domains, e.g., two Fc domains that are each individually part of a separate antibody heavy chain. The first and second multimerization domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerization domain is an Fc fragment or an amino acid sequence of from 1 to about 200 amino acids in length containing at least one cysteine residue. In other embodiments, the multimerization domain is a cysteine residue, or a short cysteine-containing peptide. Other multimerization domains include peptides or polypeptides comprising or consisting of a leucine zipper, a helix-loop motif, or a coiled-coil motif.

The multispecific molecules of the present invention may include multimerization domains, e.g., Fc domains, comprising one or more amino acid changes (e.g., insertions, deletions or substitutions) as compared to the wild-type, naturally occurring version of the Fc domain. For example, the invention includes multispecific molecules comprising one or more modifications in the Fc domain that results in a modified Fc domain having a modified binding interaction (e.g., enhanced or diminished) between Fc and FcRn. In one embodiment, the multispecific molecule comprises a modification in a CH2 or a CH3 region, wherein the modification increases the affinity of the Fc domain to FcRn in an acidic environment (e.g., in an endosome where pH ranges from about 5.5 to about 6.0). Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

The present invention also includes multispecific molecules comprising a first multimerization domain and a second multimerization domain (e.g., Ig Fc domains), wherein the first and/or second multimerization domains comprise an amino acid sequence which facilitates purification of the multispecific molecule.

In one embodiment, the amino acid sequence which facilitates purification of the multispecific molecule is an amino acid substitution that leads to a weak or no detectable binding to an Fc-binding affinity matrix. In one specific embodiment, one of the two multimerization domains comprises a CH3 domain that is capable of binding to Protein A (“Fc”) and the other of the two multimerization domains comprises a CH3 domain that is not capable of binding to Protein A (“Fc*”). In some embodiments, the second multimerization domain comprises a H435R/Y436F (by EU numbering system; H95R/Y96F by IMGT exon numbering system) substitution in its CH3 domain (“Fc*” or “star substitution”) and exhibits weak or no detectable binding to Fc-binding ligands, such as protein A, protein G, protein L, or derivatives thereof. The three-component mixture of FcFc* heterodimer and the FcFc and Fc*Fc* homodimers can be separated using the differential binding affinity chromatography. See, for example, U.S. Pat. No. 8,586,713. Further modifications that may be found within the second CH3 include, e.g., D16E, L18M, N44S, K52N, V57M, and V82I (by IMGT; D356E, L358M, N384S, K392N, V397M, and V422I by EU) in the case of IgG1 heavy chains; N44S, K52N, and V82I (IMGT; N384S, K392N, and V422I by EU) in the case of IgG2 heavy chains; and Q15R, N44S, K52N, V57M, R69K, E79Q, and V82I (by IMGT; Q355R, N384S, K392N, V397M, R409K, E419Q, and V422I by EU) in the case of IgG4 heavy chains. Additional examples, where an additional mutation that abrogates binding to Affinity Columns is a mutation to the Heavy chain Variable Region (VH) on the same chain with the Fc* mutation, are provided in, e.g., U.S. Pat. No. 9,493,563 (mutations in VH3 and Fc described as IMGT 3, 5, 7, 20, 22, 26, 27, 79, 81, 84, 84.2, 85.1, 86, 90).

In certain embodiments, the multispecific molecules of the invention comprise a substitution of the protein-protein interface between the CH3 domains of the antibody Fc region with the protein-protein interface found in the T-cell receptor (TCR) constant region. Lc mispairing is avoided by the replacement of one Fab arm of the bispecific IgG by a scFv. For purification purposes the molecule is designed with a missing Protein A binding site on the Hc of the molecule. Consequently, homodimeric molecules harboring 2 Hc do not bind to the Protein A column, while the heterodimeric molecule and the homodimeric Fc-scFv molecule exhibit a different affinity for Protein A as these molecules harbor one and two binding sites for Protein A, respectively. See, e.g., U.S. Pat. Nos. 9,683,052 and 9,683,053 and U.S. Pat. Appl. Pub. No. 20150239991.

In certain embodiments, the multispecific molecules of the invention comprise a knob-into-holes pair created by the amino acid changes of T22Y in strand B of the first CH3 domain and Y86T in strand E of the partner CH3 domain. The T22Y amino acid change creates the knob, while Y86T, in the partner CH3 domain, creates the hole. See, e.g., Ridgway, J. B. et al. Protein Eng. 9(7):617-2 (1996).

In certain embodiments, the Fc domain may be chimeric, combining Fc sequences derived from more than one immunoglobulin isotype. For example, a chimeric Fc domain can comprise part or all of a CH2 sequence derived from a human IgG1, human IgG2 or human IgG4 CH2 region, and part or all of a CH3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 CH1]-[IgG4 upper hinge]-[IgG2 lower hinge]-[IgG4 CH2]-[IgG4 CH3]. Another example of a chimeric Fc domain that can be included in any of the multispecific molecules set forth herein comprises, from N- to C-terminus: [IgG1 CH1]-[IgG1 upper hinge]-[IgG2 lower hinge]-[IgG4 CH2]-[IgG1 CH3]. These and other examples of chimeric Fc domains that can be included in any of the multispecific molecules of the present invention are described in US Publication 2014/0243504, published Aug. 28, 2014, which is herein incorporated in its entirety. Chimeric Fc domains having these general structural arrangements, and variants thereof, can have altered Fc receptor binding.

In certain embodiments, the invention provides a multimerization domain that is an antibody heavy chain wherein the heavy chain constant region (CH) region comprises an amino acid sequence at least 95%, at least 96%, at least 97%, at least 98%, at least 99% identical to any wildtype allele of human IgG1, IgG2, IgG3, IgG4, IgA, IgD, IgE, IgM, or IgY.

### Binding Properties of the Multispecific Molecules of the Invention

As used herein, the term “binding” in the context of the binding of a domain that specifically binds a molecule expressed on the surface of the cell (e.g., T-cell or B-cell), an antigen-binding domain, or an antibody-binding fragment to either, e.g., a predetermined antigen, such as a cell surface protein or fragment thereof, typically refers to an interaction or association between a minimum of two entities or molecular structures, such as an antibody-antigen interaction.

For instance, binding affinity typically corresponds to a KD value of about 10-7 M or less, such as about 10-8 M or less, such as about 10-9 M or less when determined by, for instance, surface plasmon resonance (SPR) technology in a BIAcore 3000 instrument using the antigen as the ligand and the antigen-binding domain as the analyte (or anti-ligand). Cell-based binding strategies, such as fluorescent-activated cell sorting (FACS) binding assays, are also routinely used, and FACS data correlates well with other methods such as radioligand competition binding and SPR (Benedict, CA, J Immunol Methods. 1997, 201(2):223-31; Geuijen, CA, et al. J Immunol Methods. 2005, 302(1-2):68-77).

Accordingly, domains that specifically binds a molecule expressed on the surface of the cell or antigen-binding domains of the invention bind to the predetermined antigen or cell surface molecule (receptor) having an affinity corresponding to a KD value that is at least ten-fold lower than its affinity for binding to a non-specific antigen (e.g., BSA, casein). According to the present invention, the affinity of a domain that specifically binds a molecule expressed on the surface of the cell or an antigen-binding domain corresponding to a KD value that is equal to or less than ten-fold lower than a non-specific antigen may be considered non-detectable binding.

The term “KD” (M) refers to the dissociation equilibrium constant of a particular domain that specifically binds a molecule expressed on the surface of the cell or antigen-binding domain-antigen interaction, or the dissociation equilibrium constant of a domain that specifically binds a molecule expressed on the surface of the cell or an antigen-binding domain binding to an antigen. There is an inverse relationship between KD and binding affinity, therefore the smaller the KD value, the higher, i.e. stronger, the affinity. Thus, the terms “higher affinity” or “stronger affinity” relate to a higher ability to form an interaction and therefore a smaller KD value, and conversely the terms “lower affinity” or “weaker affinity” relate to a lower ability to form an interaction and therefore a larger KD value. In some circumstances, a higher binding affinity (or KD) of a particular molecule (e.g. one-arm antibody) to its interactive partner molecule (e.g. antigen X) compared to the binding affinity of the molecule (e.g. one-arm antibody) to another interactive partner molecule (e.g. antigen Y) may be expressed as a binding ratio determined by dividing the larger KD value (lower, or weaker, affinity) by the smaller KD (higher, or stronger, affinity), for example expressed as 5-fold or 10-fold greater binding affinity, as the case may be.

The term “kd” (sec−1 or 1/s) refers to the dissociation rate constant of a particular one-arm antibody-antigen interaction, or the dissociation rate constant of a one-arm antibody or antibody-binding fragment. Said value is also referred to as the koff value.

The term “ka” (M−1×sec−1 or 1/M) refers to the association rate constant of a particular one-arm antibody-antigen interaction, or the association rate constant of a one-arm antibody or antibody-binding fragment.

The term “KA” (M−1 or 1/M) refers to the association equilibrium constant of a particular one arm antibody-antigen interaction, or the association equilibrium constant of a one-arm antibody or antibody-binding fragment. The association equilibrium constant is obtained by dividing the ka by the kd.

The term “EC50” or “EC50” refers to the half maximal effective concentration, which includes the concentration of a multispecific molecule, a pMHC complex, and/or a molecule that binds specifically to a molecule expressed on the surface of a cell (e.g., T-cell) which induces a response halfway between the baseline and maximum after a specified exposure time. The EC50 essentially represents the concentration of a multispecific molecule, a pMHC complex, and/or a molecule that binds specifically to a molecule expressed on the surface of a cell (e.g., T-cell) where 50% of its maximal effect is observed.

In one embodiment, the EC50 value represents the concentration of a multispecific molecule, a pMHC complex, and/or a molecule that binds specifically to a molecule expressed on the surface of a cell (e.g., T-cell) of the invention that elicits half-maximal depletion of target cells by T cell cytotoxic activity. Thus, increased cytotoxic activity (e.g. T cell-mediated tumor cell killing) is observed with a decreased EC50, or half maximal effective concentration value.

T-Cell Modulating Characteristics of the Multispecific Molecules

The multispecific molecules of the present invention are useful for modulating an activity of a T cell with specificity for the peptide component of the first binding molecule. A T cell with specificity for the peptide presented in the groove of the MHC domain components of the first binding molecule will bind the peptide/MHC complex via a T cell receptor (each T cell has approximately 30,000 TCRs, each of which comprises variable domains similar to the antigen-binding domains of an antibody). T cell activation or suppression is accomplished based on the specificity of the antigen-binding domain of the second binding molecule. In some embodiments, the second binding molecule comprises an antigen-binding domain that specifically binds a co-stimulatory molecule on the T cell (e.g., CD28) to provide a signal to induce activation, proliferation and/or survival of the T cell. In other embodiments, the second binding molecule comprises an antigen-binding domain that specifically binds a inhibitory molecule on the T cell (e.g., LAG3) to provide a signal to suppress activation, or to induce anergy or T-cell death. In some embodiments, modulation of T cell activity is accomplished in vivo by administration of a multispecific molecule of the present invention to a subject in need thereof. The subject in need thereof may have, or be at higher risk of, a disease or disorder that can be prevented, treated or ameliorated by modulating T cell activity. For example, the subject may have, or be at elevated risk of, an infection, cancer, or an autoimmune disorder. In some embodiments, modulation of T cell activity is accomplished ex vivo. In various embodiments, modulation of T cell activity ex vivo may be performed by obtaining T cells (CD4+ or CD8+) from a subject, and culturing the T cells with a plurality of multispecific molecules of the present invention under conditions and for a period of time sufficient to modulate the activity of the T cells.

In some embodiments, the multispecific molecules of the present disclosure (e.g., bispecific molecules) can be used ex vivo to modulate (e.g., induce activation or anergy) autologous T cells for use in the treatment of diseases or disorders amenable to T cell modulation (e.g., cancers, infectious diseases, or autoimmune disorders). For example, CD8+ and/or CD4+ T cells can be obtained from a subject via apheresis, cultured with the multispecific molecules discussed herein under conditions to facilitate activation and proliferation of the T cells (e.g., CD8+ T cells), or to induce anergy in the T cells (e.g., CD4+ T cells), followed by reintroduction of the T cells into the subject. As part of the culturing process, T cells can be selected to enrich the proportion of cells with specificity for the peptide (PiG) of the MHC component of the multispecific molecules. In some embodiments, the subject is a cancer patient, and the PiG comprises a fragment of a tumor-associated antigen. Autologous T cells (e.g., CD8+ T cells) are removed from the patient via apheresis, cultured with the multispecific molecules discussed herein (e.g., bispecific molecules comprising a pMHC complex displaying a peptide in a class I MHC polypeptide, and an anti-CD28 binding domain) under conditions to induce activation and proliferation of the T cells with a specificity for the peptide, followed by reintroduction of the activated/proliferated T cells into the patient. In some embodiments, the patient is an individual suffering from an infectious disease, and autologous T cells are removed, cultured, and reintroduced in a similar manner, except that the PiG comprises a fragment of an infectious disease antigen associated with the patient's infection.

### Clustering

In some embodiments, clustering the T cells in proximity to one another can enhance the modulatory activity of the multispecific molecules or multispecific carrier molecules of the present invention. In some embodiments, clustering includes bringing 4 or more T cells into proximity with one another such that they can bind cytokines secreted by neighboring T cells. In some embodiments, clustering includes bringing about 1200 or more T cells into proximity with one another such that they can bind cytokines secreted by neighboring T cells. In various embodiments, the number of T cells “clustered,” as discussed herein, may be about 5, about 10, about 15, about 20, about 25, about 30, about 40, about 50, about 75, about 100, about 500, about 1000, about 5000, about 6000, about 7000 or about 8000 or more. In some embodiments, clustering includes bringing together about 1000 or more T cells in the presence of an antigen recognized by the TCR, and a co-stimulatory signal. In some embodiments, clustering includes bringing together about 1200 or more T cells in the presence of an antigen recognized by the TCR, and a co-stimulatory signal. In some embodiments, clustering includes bringing together 1500 or more T cells in the presence of an antigen recognized by the TCR, and a co-stimulatory signal.

In an in vivo environment, clustering may be achieved, for example, via binding of the first molecule or multispecific molecule to a carrier (e.g., cell such as a B-cell, viral like particles etc. . . . ), thereby bringing the TCRs and/or co-stimulatory molecules into close proximity to one another as they bind to the first molecule and/or multispecific molecules gathered on the carrier. By way of example, the Fc domain (e.g., IgG1 or IgG4) of the first molecule or multispecific molecule can be, for example, to Fcγ receptors (FcγI, FcγIIA, FcγIIB, FcγIIIA, or FcγIIIB) on a cell (e.g., a B cell). In one embodiment, the first molecules or multispecific molecules of the present invention may comprise a domain (e.g., an Fc domain) that comprises or is fused to an antigen-binding domain that specifically binds a cell surface molecule. In certain embodiments, the antigen binding domain is linked to the C-terminus of the first molecule, second molecule, and/or multimerization domain. Non-limiting examples of this second antigen-binding domain can have specificity for a surface molecule (e.g., CD5, CD19, CD20, CD21, CD22, CD23, CD24, CD25, CD27, CD30, CD34, CD35, CD38, CD180, or CD40) or a tumor-associated antigen (e.g., as defined herein). Binding of this second antigen-binding domain to its antigen on a cell surface brings together a cluster of multispecific and/or first molecules and the T cells bound thereto. In various embodiments, the second antigen-binding domain may be a Fab or a scFv (which is alternatively referred to herein as “a Stahl body” when linked to the C-terminus of the antigen-binding domain).

In one embodiment, the second antigen-binding domain specifically binds to CD20. CD20 (a.k.a Bp35, MS4A1, LEU-16, or CVID6) (HGNC(7315), Entrez Gene(931), Ensembl(ENSG00000156738), OMIM(112210), UniProtKB(P11836), each of which are incorporated herein in their entirety) is expressed on the surface of all B-cells beginning at the pro-B phase (CD45R+, CD117+) and progressively increasing in concentration until maturity. CD20 is the target of monoclonal antibodies rituximab, ocrelizumab, obinutuzumab, ofatumumab, ibritumomab tiuxetan, tositumomab, and ublituximab.

In one embodiment, the second antigen-binding domain specifically binds to CD180, CD180 (a.k.a. Bgp-95, LY64, Ly78, or RP105; HGNC (6726), Entrez Gene (4064), Ensembl (ENSG00000134061), OMIM (602226), UniProtKB (Q99467), NP_005573.1, each of which are incorporated herein in their entirety) belongs to the family of pathogen receptors, Toll-like receptors (TLR) and is a cell surface molecule consisting of extracellular leucine-rich repeats (LRR) and a short cytoplasmic tail. See also Miura et al., Genomics 38:299-304, 1996 and Miura et al., Blood 92:2815-2822, 1998, both of which are incorporated herein in their entirety for all intended purposes. CD180 is expressed on antigen presenting cells (e.g., B cells and dendritic cells). Anti-CD180 antibodies include RP/14, MHR73, MHR73-11, and G28-8.

In other cases, clustering in an in vivo environment may be achieved, for example, by delivering the components of the multispecific molecules discussed herein (e.g., the scGP-33-MHC and the anti-CD28 binding domain) arrayed on the surface of a carrier molecule. For example, a carrier molecule can include a surface array of pMHC complex and molecules comprising an anti-T cell surface molecule binding domain. The pMHC complex can be any of such molecules discussed herein (e.g., the peptide can be derived from a tumor-associated antigen), and the anti-T cell surface molecule binding domain can be any of such molecules discussed herein (e.g., anti-CD28 or anti-PD1). In some embodiments, the carrier can be a virus-like particle (VLP) generated by overexpressing the surface proteins of interest (e.g., anti-CD28 and scMHC peptide) in production cells and harvesting the VLPs. An exemplary VLP comprising an array of scMHC-gp33 and anti-CD28 binding domains is discussed in Example 10.

In other embodiments, in vivo clustering can be achieved by expressing the molecules of the multispecific molecule on the surface of an engineered cell and introducing the engineered cell into a subject. For example, a subject's cells (e.g., B cells) can be harvested and engineered via transfection with RNA and/or DNA or transduction via a vector (e.g., a lentiviral vector) to express a first transmembrane polypeptide comprising an extracellular pMHC complex, and a second transmembrane polypeptide comprising an extracellular antigen-binding domain specific for a T-cell surface molecule (e.g., CD28 or LAG3). In some embodiments, the second transmembrane polypeptide can be the antigen-binding domain of an antibody, for example, an scFv comprising the light chain variable region (LCVR) and heavy chain variable region (HCVR) of an antibody (e.g., an anti-CD28 antibody) linked to a transmembrane domain to anchor the polypeptide to the cell surface. In practice, a plurality of the first and second transmembrane polypeptides are expressed on the surface of the engineered cells (e.g., B cells) such that reintroduction of the population of cells to the subject will enable binding and clustering of a plurality of peptide-specific T cells to promote modulation of T cell activity (e.g., activation of the T cells).

In an ex vivo environment, clustering may be accomplished by artificially arraying the multispecific molecules in a manner that brings together groups of T cells bound to the multispecific molecules. In one embodiment, the plurality of multispecific molecules may be bound to a scaffold in a clustered arrangement in the culture. A “clustered arrangement,” as used herein, refers to an arrangement of the multispecific molecules in such proximity to one another that bound T cells are able to bind cytokines (e.g., IL-2) secreted by neighboring T cells. In another embodiment, the plurality of multispecific molecules may be clustered via one or more linkers. In some embodiments, the linkers used for clustering the multispecific molecules are multivalent antibodies with specificity for a portion of the Fc domains of the multispecific molecules. In one embodiment, the linkers (e.g., multivalent antibodies) are provided in a ratio of 1:1 with the multispecific molecules. In other embodiments, the ratio of linker (e.g., multivalent antibody) to multispecific molecule is 5:1, 4:1, 3:1, 2:1, 1:2, 1:3, 1:4, or 1:5. In another embodiment, the second antigen-binding domain (e.g., a Fab or scFv) contained in or fused to the Fc domain of the multispecific molecules, as discussed above, can be used in an ex vivo environment in which the culture includes cells expressing the antigen specific to the second antigen-binding domain or a scaffold including the antigen.

### B Cell Preparation/Activation

The present invention also provides methods for making the cells (e.g., B cells) which express the first and second transmembrane polypeptides as described herein. In one embodiment, the method comprises transfecting or transducing cells isolated from a subject. In certain embodiments, the cells are isolated from an individual and genetically modified without further manipulation in vitro. In certain embodiments, the B cells are mature B cells. In certain embodiments, the cells can then be directly re-administered into the individual once engineered.

In further embodiments, the cells are first stimulated/activated to proliferate in vitro prior to being genetically modified to express the transmembrane polypeptides. In this regard, the cells may be cultured before or after being genetically modified (i.e., transduced or transfected to express the transmembrane polypeptides as described herein). In certain examples, the increased activity may be at a level of two, three, four, five, six, seven, eight, nine, or tenfold, or more, than that of the non-contacted cell, or the cell contacted with the negative control.

In certain embodiments, the B cell activating factors may be attached to the C-terminus of the first and/or second molecule.

In certain embodiments, the B cells are activated via peptides and/or antigen-binding domains that specifically bind a B-cell surface molecule. In some embodiments, the B-cell surface molecule is CD5, CD19, CD20, CD21, CD22, CD23, CD24, CD25, CD27, CD30, CD34, CD35, CD38, CD180, CD40, Toll-like receptors (TLRs) (e.g., TLR1, TLR2, TLR3, TLR4, TLR5, TLR6, TLR7, TLR8, TLR9, TLR10, TLR11, TLR12, and TLR13), C-type lectin receptors (CLRs), or interleukin-1 receptors. In certain embodiments, the peptides and/or antigen-binding domains that specifically bind a B-cell surface molecule may be attached to the C-terminus of the first and/or second molecule. In various embodiments, the antigen-binding domains that specifically bind a B-cell surface molecule may be a Fab or a scFv (which is alternatively referred to herein as “a Stahl body”).

In certain embodiments, activation of B cells is augmented by inducible adapters such as, but not limited to, inducible PRR adapters include (e.g., MyD88 (including truncated forms such as those lacking the TIR domain) and TRIF), inducible Pattern Recognition Receptors (e.g., NOD-like receptors, such as NOD1 or NOD2), RIG-like helicases, (e.g., RIG-I or Mda-5), and CD40 cytoplasmic domain.

Prior to in vitro manipulation or genetic modification of the cells (e.g., B cells) described herein, the source of cells may be obtained from a subject. In one embodiment, cells from the circulating blood of an individual are obtained by apheresis. The apheresis product typically contains lymphocytes, including T cells, monocytes, granulocyte, B cells, other nucleated white blood cells, red blood cells, and platelets. In one embodiment, the cells collected by apheresis may be washed to remove the plasma fraction and to place the cells in an appropriate buffer or media for subsequent processing. In one embodiment of the invention, the cells are washed with PBS. In an alternative embodiment, the washed solution lacks calcium, and may lack magnesium or may lack many, if not all, divalent cations. As would be appreciated by those of ordinary skill in the art, a washing step may be accomplished by methods known to those in the art, such as by using a semiautomated flowthrough centrifuge. After washing, the cells may be resuspended in a variety of biocompatible buffers or other saline solution with or without buffer. In certain embodiments, the undesirable components of the apheresis sample may be removed in the cell directly resuspended culture media.

Engineering Cells to Express First and/or Second Molecules

The transmembrane polypeptides of the present invention are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extra chromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding a transmembrane polypeptide carried by a retroviral vector can be transduced into a cell through infection and pro virus integration.

As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably.

In certain embodiments, the nucleic acid or viral vector is transferred via ex vivo transformation. Methods for transfecting vascular cells and tissues removed from an organism in an ex vivo setting are known to those of skill in the art. Thus, it is contemplated that cells or tissues may be removed and transfected ex vivo using the polynucleotides presented herein. In particular aspects, the transplanted cells or tissues may be placed into an organism. Thus, it is well within the knowledge of one skilled in the art to isolate antigen-presenting cells (e.g., B cells) from an animal (e.g., human), transfect the cells with the expression vector and then administer the transfected or transformed cells back to the animal.

In certain embodiments, the nucleic acid or viral vector is transferred via injection. In certain embodiments, a polynucleotide may be delivered to an organelle, a cell, a tissue or an organism via one or more injections (i.e., a needle injection), such as, for example, subcutaneously, intradermally, intramuscularly, intravenously, intraperitoneally, etc. Methods of injection of vaccines are well known to those of ordinary skill in the art (e.g., injection of a composition comprising a saline solution). Further embodiments include the introduction of a polynucleotide by direct microinjection. The amount of the expression vector used may vary upon the nature of the antigen as well as the organelle, cell, tissue or organism used.

In certain embodiments, a polynucleotide is introduced into an organelle, a cell, a tissue or an organism via electroporation. Electroporation involves the exposure of a suspension of cells and DNA and/or RNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference).

In certain embodiments, a polynucleotide is delivered into a cell using DEAE-dextran followed by polyethylene glycol (see e.g., Gopal, T. V., Mol Cell Biol. 1985 May; 5(5):1188-90), sonication loading (see e.g., Fechheimer et al., (1987) Proc. Nat'l Acad. Sci. USA, 84, 8463-8467), liposome-mediated transfection, receptor mediated delivery vehicles transfection (see e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085), and/or microprojectile bombardment (see e.g., U.S. Pat. Nos. 5,550,318; 5,538,880; 5,610,042; and PCT Application WO 94/09699). Each reference listed in this paragraph is incorporated herein by reference in their entirety for all intended purposes.

In certain embodiments, the polynucleotides encoding the first and second transmembrane polypeptides described herein are inserted into a vector or vectors. The vector is a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well. An expression vector may comprise additional elements, for example, the expression vector may have two replication systems, thus allowing it to be maintained in two organisms, for example in human cells for expression and in a prokaryotic host for cloning and amplification.

The expression vector may have the necessary 5′ upstream and 3′ downstream regulatory elements such as promoter sequences such as CMV, PGK and EF1alpha. promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing the transmembrane polypeptides. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector may have additional sequence such as 6x-histidine (SEQ ID NO: 29), c-Myc, and FLAG tags which are incorporated into the expressed polypeptides. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector may also be engineered for replication and/or expression functionality (e.g., transcription and translation) in a particular cell type, cell location, or tissue type. Expression vectors may include a selectable marker for maintenance of the vector in the host or recipient cell.

In various embodiments, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-XTM Bicistronic Expression System (Neo) vectors (Clontech), pClneo vectors (Promega) for expression in mammalian cells; pLenti4/V5-DEST.TM., pLenti6/V5-DEST.TM., and pLenti6.2N5-GW/lacZ (Invitrogen) for lentivirus-mediated gene transfer and expression in mammalian cells. The coding sequences of the transmembrane polypeptides disclosed herein can be ligated into such expression vectors for the expression of the chimeric protein in mammalian cells.

In certain embodiments, the nucleic acids encoding the transmembrane polypeptides of the present invention are provided in a viral vector. A viral vector can be that derived from retrovirus, lentivirus, or foamy virus. As used herein, the term, “viral vector,” refers to a nucleic acid vector construct that includes at least one element of viral origin and has the capacity to be packaged into a viral vector particle. The viral vector can contain the coding sequence for the various chimeric proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the viral vector containing the coding sequence for the transmembrane polypeptides described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MOMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). Retroviruses” of the invention also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72:8463-8471 and Zufferey et al., J. Virol. 1998; 72:9873-9880).

Retroviral vectors (i.e., both lentiviral and non-lentiviral) for use in the present invention can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of the first and second transmembrane polypeptides, the vector or vectors may be introduced into a host cell to allow expression of the polypeptides within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells (e.g., B cells) containing the vectors provided herein. The host cells containing the vector may be useful in expression or cloning of the polynucleotide contained in the vector.

### Sequence Variants

The antigen-binding domains of the multispecific molecules of the present invention may comprise one or more amino acid substitutions, insertions and/or deletions in the framework and/or CDR regions of the heavy and light chain variable domains as compared to the corresponding germline sequences from which the individual antigen-binding domains were derived. Such mutations can be readily ascertained by comparing the amino acid sequences disclosed herein to germline sequences available from, for example, public antibody sequence databases. The antigen-binding domains may be derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antigen-binding domains and antigen-binding fragments which comprise one or more individual germline mutations or combinations thereof. In certain embodiments, all of the framework and/or CDR residues within the VH and/or VL domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, etc. Multispecific molecules comprising one or more antigen-binding domains obtained in this general manner are encompassed within the present invention.

The present invention also includes antigen-binding domains comprising variants of any of the HCVR, LCVR, and/or CDR amino acid sequences disclosed herein having one or more conservative substitutions. For example, the present invention includes antigen-binding domains having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

The present invention also includes antigen-binding domains with an HCVR, LCVR, and/or CDR amino acid sequence that is substantially identical to any of the HCVR, LCVR, and/or CDR amino acid sequences disclosed herein. The term “substantial identity” or “substantially identical,” when referring to an amino acid sequence means that two amino acid sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

### Preparation of Antigen-Binding Domains and Construction of Multispecific Molecules

Antigen-binding domains specific for particular antigens (e.g., CD28 or CTLA-4) can be prepared by any antibody generating technology known in the art. In certain embodiments, one or more of the individual components (e.g., heavy and light chains) of the antigen-binding domains of the invention are derived from chimeric, humanized or fully human antibodies. Methods for making such antibodies are well known in the art. For example, one or more of the heavy and/or light chains of the antigen-binding domains of the multispecific molecules of the present invention can be prepared using VELOCIMMUNE™ technology. Using VELOCIMMUNE™ technology (or any other human antibody generating technology), high affinity chimeric antibodies to a particular antigen (e.g., CD28 or CTLA-4) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the multispecific molecules of the present invention.

Genetically engineered animals may be used to make human antigen-binding domains. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human antigen-binding domains. Fully human refers to an antibody, or antigen-binding domain, or fragment thereof, comprising an amino acid sequence encoded by a DNA derived from a human sequence over the entire length of each polypeptide of the antibody or antigen-binding domain or fragment thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g. compared to any wild-type human immunoglobulin regions or domains.

Once obtained, the antigen-binding domain can be appropriately arranged relative to the peptide-MHC polypeptide components and the multimerization domains to produce a multispecific molecule of the present invention using routine methods, e.g., as discussed in Example 1.

### Bioequivalents

The present invention encompasses multispecific molecules having amino acid sequences that vary from those of the exemplary molecules disclosed herein but that retain the ability to bind a specific antigen (e.g., CD28 or CTLA-4) and specific T cell receptors. Such variant molecules may comprise one or more additions, deletions, or substitutions of amino acids when compared to a parent sequence, but exhibit biological activity that is essentially equivalent to that of the described multispecific molecules.

The present invention includes multispecific molecules that are bioequivalent to any of the exemplary multispecific molecules (including antigen-binding domains and peptide-MHC fusion polypeptides) set forth herein. Two such multispecific molecules, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some multispecific molecules will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two multispecific molecules are bioequivalent if there are no clinically meaningful differences in their safety, purity, and potency.

In one embodiment, two multispecific molecules are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two multispecific molecules are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the multispecific molecule or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the multispecific molecule (or its target) is measured as a function of time; and (d) in a well-controlled clinical trial that establishes safety, efficacy, or bioavailability or bioequivalence of a multispecific molecule.

Bioequivalent variants of the exemplary multispecific molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent multispecific molecules may include variants of the exemplary multispecific molecules (including antigen-binding domains, peptide-MHC polypeptides, and multimerization domains) set forth herein comprising amino acid changes which modify the glycosylation characteristics of the molecules, e.g., mutations which eliminate or remove glycosylation.

### Therapeutic Formulation and Administration

The present invention provides pharmaceutical compositions comprising the multispecific molecules of the present invention. The pharmaceutical compositions of the invention are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of multispecific molecule administered to a patient may vary depending upon the age and the size of the patient, target disease, conditions, route of administration, and the like. The preferred dose is typically calculated according to body weight or body surface area. When a multispecific molecule of the present invention is used for therapeutic purposes in an adult patient, it may be advantageous to intravenously administer the multispecific molecule of the present invention normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a multispecific molecule may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the multispecific molecule or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid multispecific molecule contained is generally about 5 to about 500 mg per dosage form in a unit dose; especially in the form of injection, it is preferred that the aforesaid multispecific molecule is contained in about 5 to about 100 mg and in about 10 to about 250 mg for the other dosage forms.

### Therapeutic Uses of the Multispecific Molecules

The present invention includes methods comprising administering to a subject in need thereof a therapeutic composition comprising a multispecific molecule as discussed herein. The therapeutic composition can comprise any of the multispecific molecules as disclosed herein and a pharmaceutically acceptable carrier or diluent. As used herein, the expression “a subject in need thereof” means a human or non-human animal that exhibits one or more symptoms or indicia of an infection (e.g., a subject suffering from a bacterial or viral infection, including any of those mentioned herein) cancer (e.g., a subject expressing a tumor or suffering from any of the cancers mentioned herein), an autoimmune disorder (e.g., a subject suffering from any of the autoimmune diseases or disorders mentioned herein), inflammatory diseases, or who otherwise would benefit from enhancement or suppression of T cell activity.

In another aspect, described herein is a method of treating a disorder in a subject in need thereof comprising administering to said subject an effective amount of a multispecific molecule described herein, wherein the multispecific molecule binds to an antigen-specific TCR and wherein the antigen recognized by the TCR is associated with the disorder.

The multispecific molecules of the invention (and therapeutic compositions comprising the same) are useful, inter alia, for treating any disease or disorder in which stimulation or suppression of an immune response (via T cell modulation) targeted against a specific antigen would be beneficial. In particular, the multispecific molecules of the present invention may be used for the treatment and prevention of infections, cancers or autoimmune disorders.

Where the multispecific molecule described herein includes a second molecule comprising a domain that specifically binds a T-cell surface molecule that is an activating polypeptide, transduction of the T cell with the multispecific molecule activates the epitope-specific T cell. In some instances, the epitope-specific T cell is a T cell that is specific for an epitope present on a cancer cell, and contacting the epitope-specific T cell with the multispecific molecule increases cytotoxic activity of the T cell toward the cancer cell. In some embodiments, the epitope-specific T cell is a T cell that is specific for an epitope present on a cancer cell, and contacting the epitope-specific T cell with the multispecific molecule increases the number of the epitope-specific T cells.

In some embodiments, the epitope-specific T cell is a T cell that is specific for an epitope present on a virus-infected cell, and contacting the epitope-specific T cell with the multispecific molecule increases cytotoxic activity of the T cell toward the virus-infected cell. In some instances, the epitope-specific T cell is a T cell that is specific for an epitope present on a virus-infected cell, and contacting the epitope-specific T cell with the multispecific molecule increases the number of the epitope-specific T cells.

Where the multispecific molecule described herein includes a second molecule comprising a domain that specifically binds a T-cell surface molecule that is an inhibiting polypeptide, contacting the T cell with the multispecific molecule inhibits the epitope-specific T cell. In some instances, the epitope-specific T cell is a self-reactive T cell that is specific for an epitope present in a self antigen, and the contacting reduces the number of the self-reactive T cells.

The interaction of a T cell with the multispecific molecules described herein can result in, e.g., activation, induction of anergy, or death of a T cell that occurs when the TCR of the T cell is bound by a TCR-binding pMHC complex. “Activation of a T cell” refers to induction of signal transduction pathways in the T cell resulting in production of cellular products (e.g., interleukin-2) by that T cell. “Anergy” refers to the diminished reactivity by a T cell to an antigen. Activation and anergy can be measured by, for example, measuring the amount of IL-2 produced by a T cell after an pMHC complex has bound to the TcR. Anergic cells will have decreased IL-2 production when compared with stimulated T cells. Another method for measuring the diminished activity of anergic T cells includes measuring intracellular and/or extracellular calcium mobilization by a T cell upon engagement of its TCR's. “T cell death” refers to the permanent cessation of substantially all functions of the T cell.

T-cell phenotypes may be evaluated using well-known methods, e.g., T cell activation may be determined, e.g., by measuring changes in the level of expression of cytokines and/or T cell activation markers, and/or the induction of antigen-specific proliferating cells. Techniques known to those of skill in the art, including, but not limited to, immunoprecipitation followed by Western blot analysis, ELISAs, flow cytometry, Northern blot analysis, and RT-PCR can be used to measure the expression cytokines and T cell activation markers. Cytokine release may be measured by measuring secretion of cytokines including but not limited to Interleukin-2 (IL-2), Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, IFN-γ, TFN-γ, IGF-I, and IGF-II (see, e.g., Isaacs et al., 2001, Rheumatology, 40:724-738; Soubrane et al., 1993, Blood, 81(1): 15-19).

T cell modulation may also be evaluated by measuring, e.g., proliferation by, e.g., 3H-thymidine incorporation, trypan blue cell counts, and fluorescence activated cell sorting (FACS).

The anti-tumor responses of T cells after exposure to the multispecific molecules described herein may be determined in xenograft tumor models. Tumors may be established using any human cancer cell line expressing the tumor associated antigen presented by the multispecific molecules. In order to establish xenograft tumor models, about 5×106 viable cells, may be injected, e.g., s.c., into nude athymic mice using for example Matrigel (Becton Dickinson). The endpoint of the xenograft tumor models can be determined based on the size of the tumors, weight of animals, survival time and histochemical and histopathological examination of the cancer, using methods known to one skilled in the art.

The anergic state or death of T cells after exposure to the multispecific molecules described herein, e.g., which may be useful for treatment of inflammatory and autoimmune disorders, can be tested in vitro or in vivo by, e.g., 51Cr-release assays. The ability to mediate the depletion of peripheral blood T cells can be assessed by, e.g., measuring T cell counts using flow cytometry analysis.

Non-limiting examples of useful animal models for analyzing the effect of the exposure of T cells to the multispecific molecules described herein on inflammatory diseases include adjuvant-induced arthritis rat models, collagen-induced arthritis rat and mouse models and antigen-induced arthritis rat, rabbit and hamster models (see, e.g., Crofford L. J. and Wilder R. L., “Arthritis and Autoimmunity in Animals”, in Arthritis and Allied Conditions: A Textbook of Rheumatology, McCarty et al. (eds.), Chapter 30 (Lee and Febiger, 1993); Trenthorn et al., 1977, J. Exp. Med. 146:857; Courtenay et al., 1980, Nature 283:665; Cathcart et at, 1986, Lab. Invest. 54:26; Holmdahl, R., 1999, Curr. Biol. 15: R528-530). Other useful animal models of inflammatory diseases include animal models of inflammatory bowel disease, ulcerative colitis and Crohn's disease induced, e.g., by sulfated polysaccharides (e.g., amylopectin, carrageen, amylopectin sulfate, dextran sulfate) or chemical irritants (e.g., trinitrobenzenesulphonic acid (TNBS) or acetic acid). See, e.g., Kim et al., 1992, Scand. J. Gastroentrol. 27:529-537; Strober, 1985, Dig. Dis. Sci. 30 (12 Suppl): 3S-10S).

Additional useful models are animal models for asthma such as, e.g., adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (see, e.g., Cohn et al., 1997, J. Exp. Med. 1861737-1747). Useful animal models of studying the effect of the multispecific molecules of the invention on multiple sclerosis (MS) include an experimental allergic encephalomyelitis (EAE) model (see, e.g., Zamvil et al, 1990, Ann. Rev, Immunol. 8:579). Animal models which can be used for analyzing the effect of the multispecific molecules of the invention on autoimmune disorders such as type 1 diabetes, thyroid autoimmunity, systemic lupus eruthematosus, and glomerulonephritis have been also developed (see, e.g., Bluestone et al., 2004, PNAS 101:14622-14626; Flanders et al., 1999, Autoimmunity 29:235-246; Krogh et al., 1999, Biochimie 81:511-515; Foster, 1999, Semin. Nephrol. 19:12-24).

Efficacy of the multispecific molecules disclosed herein to downregulate immune responses in treating an autoimmune disorder may be evaluated, e.g., by detecting their ability to reduce one or more symptoms of the autoimmune disorder, to reduce mean absolute lymphocyte counts, to decrease T cell activation, to decrease T cell proliferation, to reduce cytokine production, or to modulate one or more particular cytokine profiles (e.g., Interleukin-2 (IL-2). Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-β, IFN-γ, TFN-γ, IGF-I, and IGF-II) (see, e.g., Isaacs et al., 2001, Rheumatology, 40:724-738; Soubrane et al., 1993, Blood, 81 (1): 15-19).

Efficacy of the multispecific molecules for use in treating diabetes may be evaluated, e.g. by the ability of the multispecific molecules to reduce one or more symptoms of diabetes, to preserve the C-peptide response to MMTT, to reduce the level HA1 or HA1c, to reduce the daily requirement for insulin, or to decrease T cell activation in pancreatic islet tissue. Efficacy in treating arthritis may be assessed through tender and swollen joint counts, determination of a global scores for pain and disease activity, ESRICRP, determination of progression of structural joint damage (e.g., by quantitative scoring of X-rays of hands, wrists, and feet (Sharp method)), determination of changes in functional status (e.g., evaluated using the Health Assessment Questionnaire (HAQ)), or determination of quality of life changes (assessed, e.g., using SF-36).

In a related aspect, disclosed herein is a method of treating a disorder in a subject in need thereof comprising administering to said subject an effective amount of the multispecific molecule described herein, wherein the multispecific molecule binds to an antigen-specific TCR and wherein the antigen is associated with the disorder. In some embodiments, the disorder an inflammatory or an autoimmune disorder and the administration results in a downregulation of an inflammatory or autoimmune response. In one specific embodiment, the disorder is celiac disease or gluten sensitivity. In one specific embodiment, the antigen comprises a gliadin or a fragment thereof (e.g., (i) α-gliadin fragment corresponding to amino acids 57-73 or (ii) γ-gliadin fragment corresponding to amino acids 139-153 or (iii) ω-gliadin fragment corresponding to amino acids 102-118). In one specific embodiment, the multispecific molecule presents a peptide derived from the antigen in the context of a class II MHC. In some embodiments, the disorder is a tumor and the administration results in an upregulation of an anti-tumor immune response. In another embodiment, the disorder is an infection caused by an infectious agent and the administration results in an upregulation of an immune response against the infectious agent. In one specific embodiment, the infectious agent is selected from the group consisting of a virus, a bacterium, a fungus, a protozoa, a parasite, a helminth, and an ectoparasite. In one specific embodiment, the infectious agent is lymphocytic choriomeningitis virus (LCMV) and the antigen is gp33 protein. In one specific embodiment, the multispecific molecule presents a peptide derived from the antigen in the context of a class I MHC. In some embodiments, the subject is a mammal (e.g., human).

According to certain aspects, the multispecific molecules of the present invention may be used to treat a cancer in which the tumor cells express a tumor-associated antigen, for example, a tumor-associated antigen selected from the group consisting of adipophilin, AIM- 2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1,-A2,-A3,-A4,-A6,-A9,-A10,-A12,-C1, and C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, WT-1. In some embodiments, the peptide is a neo-antigen. In some embodiments, the peptide is a tumor specific antigen. Specific cancers/tumors treatable by the methods and multispecific molecules of the present invention include, without limitation, various solid malignancies, carcinomas, lymphomas, sarcomas, blastomas, and leukemias. Non-limiting specific examples, include, for example, breast cancer, pancreatic cancer, liver cancer, lung cancer, prostate cancer, colon cancer, renal cancer, bladder cancer, head and neck carcinoma, thyroid carcinoma, soft tissue sarcoma, ovarian cancer, primary or metastatic melanoma, squamous cell carcinoma, basal cell carcinoma, brain cancers of all histopathologic types, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, testicular cancer, uterine cancer, cervical cancer, gastrointestinal cancer, mesothelioma, Ewing's tumor, leiomyosarcoma, Ewing's sarcoma, rhabdomyosarcoma, carcinoma of unknown primary (CUP), squamous cell carcinoma, basal cell carcinoma, adenocarcinoma, sweat gland carcinoma, sebaceous gland carcinoma, papillary carcinoma, Waldenstroom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, glioblastoma, astrocytoma, medulloblastoma, craniopharyngioma, ependymoma, pinealoma, hemangioblastoma, acoustic neuroma, oligodendroglioma, meningioma, retinoblastoma, leukemia, neuroblastoma, small cell lung carcinoma, bladder carcinoma, lymphoma, multiple myeloma, medullary carcinoma, B cell lymphoma, T cell lymphoma, NK cell lymphoma, large granular lymphocytic lymphoma or leukemia, gamma-delta T cell lymphoma or gamma-delta T cell leukemia, mantle cell lymphoma, myeloma, leukemia, chronic myeloid leukemia, acute myeloid leukemia, chronic lymphocytic leukemia, acute lymphocytic leukemia, hairy cell leukemia, hematopoietic neoplasias, thymoma, sarcoma, non-Hodgkin's lymphoma, Hodgkin's lymphoma, Epstein-Barr virus (EBV) induced malignancies of all types including but not limited to EBV-associated Hodgkin's and non-Hodgkin's lymphoma, all forms of post-transplant lymphomas including post-transplant lymphoproliferative disorder (PTLD), uterine cancer, renal cell carcinoma, hepatoma, hepatoblastoma, Cancers that may treated by methods and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic ameloblastoma, odontosarcoma; malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The present invention also includes methods for treating residual cancer in a subject. As used herein, the term “residual cancer” means the existence or persistence of one or more cancerous cells in a subject following treatment with an anti-cancer therapy.

Non-limiting examples of the inflammatory and autoimmune diseases include, e.g., inflammatory bowel disease (IBD), ulcerative colitis (UC), Crohn's disease, diabetes (e.g., diabetes mellitus type 1), multiple sclerosis, arthritis (e.g., rheumatoid arthritis), Graves' disease, lupus erythematosus, ankylosing spondylitis, psoriasis, Behcet's disease, autistic enterocolitis, Guillain-Barre Syndrome, myasthenia gravis, pemphigus vulgaris, acute disseminated encephalomyelitis (ADEM), transverse myelitis autoimmune cardiomyopathy, Celiac disease, dermatomyositis, Wegener's granulomatosis, allergy, asthma, contact dermatitis, atherosclerosis (or any other inflammatory condition affecting the heart or vascular system), autoimmune uveitis, as well as other autoimmune skin conditions, autoimmune kidney, lung, or liver conditions, autoimmune neuropathies, asthma, allergy, celiac disease, systemic lupus erythematosis (SLE), scleroderma, sarcoidosis, thyroiditis, multiple sclerosis, spondylitis, periarteritis, eczema, atopic dermatitis, myasthenia gravis, insulin-dependent diabetes mellitus, Crohn's disease, Guillain-Barre syndrome, Graves' disease, glomerulonephritis, ulcerative colitis, Crohn's disease, sprue, autoimmune arthritis, rheumatoid arthritis, osteoarthritis, juvenile chronic arthritis, psoriatic arthritis, reactive arthritis, spondyloarthropathy, psoriasis, acute or chronic immune disease associated with organ transplantation, an inflammatory disease, skin or organ transplantation rejection, graft-versus-host disease (GVHD), or autoimmune diseases, comprising administering to a subject a pharmaceutical composition described herein (e.g., a pharmaceutic composition comprising a multispecific molecule described herein. Examples of autoimmune diseases include, for example, glomerular nephritis, arthritis, dilated cardiomyopathy-like disease, ulcerous colitis, Sjogren syndrome, Crohn disease, systemic erythematosus, chronic rheumatoid arthritis, multiple sclerosis, psoriasis, allergic contact dermatitis, polymyositis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some embodiments of lymphopenia, mixed connective tissue disease, pemphigoid, pemphigus vulgaris, pernicious anemia, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis.

In another embodiment, the methods described herein are used for treating or preventing a transplantation-related condition. In another embodiment, the methods described herein are used for treating or preventing graft-versus-host disease. In another embodiment, the methods described herein are used for treating or preventing a post-transplant lymphoproliferative disorder.

According to certain aspects, the multispecific molecules of the present invention may be used to treat an infection, such as a bacterial infection (e.g., a bacterial infection resistant to conventional antibiotics) or a viral infection. In particular embodiments, the multispecific molecules are designed to present a peptide derived from a viral antigen or a bacterial antigen. In some embodiments, the viral antigen is derived from a virus selected from the group consisting of adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, and zika. In some embodiments, the bacterial antigen is derived from a bacterium selected from the group consisting of methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, and Clindamycin-resistant group B Streptococcus.

Multispecific molecules of the present invention designed to treat cancer or an infection may include an antigen-binding domain (e.g., a one-arm antibody) on the second binding molecule that specifically binds a T-cell co-stimulatory molecule (e.g., CD28) to induce activation, proliferation (e.g., clonal expansion) and/or survival of T cells (e.g., CD8⁺ T cells) specific for the peptide presented on the first binding molecule. In some embodiments, T cell activation is revived. In some embodiments, naïve T-cells are activated or caused to proliferate. Such T cells can enhance or stimulate an immune response against cells (e.g., tumor cells or infected cells) expressing a protein comprising the peptide presented on the first binding molecule of the multispecific molecules. In various embodiments, the multispecific molecules do not induce proliferation of non-specific T cells (i.e., T cells that are not specific for the peptide presented on the first binding molecule).

According to certain aspects, the multispecific molecules of the present invention may be used to treat, prevent, or ameliorate an autoimmune disease or disorder by targeting the activity of T cells with specificity for a peptide corresponding to an antigen associated with the autoimmune disease or disorder. For example, the antigen may be selected from the group consisting of gliadin (celiac disease; e.g., (i) α-gliadin fragment corresponding to amino acids 57-73 or (ii) γ-gliadin fragment corresponding to amino acids 139 153 or (iii) ω-gliadin fragment corresponding to amino acids 102-118), GAD 65, IA-2 and insulin β chain (for type 1-diabetes), glatiramer acetate (GA) (for multiple sclerosis), acetylcholine receptor (AChR) (for myasthenia gravis), p205, insulin, thyroid-stimulating hormone, tyrosinase, TRP1, and myelin antigens (including myelin basic protein (MBP) and proteolipid protein (PLP)). In some embodiments, the antigen may be IL-4R, IL-6R, or DLL4.

Multispecific molecules of the present invention designed to treat an autoimmune disorder may include an antigen-binding domain (e.g., a one-arm antibody) on the second binding molecule that specifically binds a T-cell inhibitory molecule (e.g., CTLA-4, LAG3, PD1, etc.) to suppress the activity of T cells (e.g., CD4+ T cells) specific for the peptide presented on the first binding molecule. Inhibition or suppression of such T cell activity can treat, alleviate, or prevent recurrence of, autoimmune diseases or disorders in which the cells targeted by the individual's immune system express a protein comprising the peptide presented on the first binding molecule of the multispecific molecule. In some embodiments, administration of the multispecific molecules of the present invention can be used to make an individual's T cells tolerant of a self-antigen for which the T cells are specific.

The present invention also includes use of the multispecific molecules discussed herein in the manufacture of a medicament for preventing, treating and/or ameliorating an infection, a cancer, or an autoimmune disorder (e.g., as discussed herein).

### Combination Therapies and Formulations

It is contemplated that when used to treat various diseases, the compositions and methods can be combined with other therapeutic agents suitable for the same or similar diseases. Also, two or more embodiments described herein may be also co-administered to generate additive or synergistic effects. When co-administered with a second therapeutic agent, the embodiment described herein and the second therapeutic agent may be simultaneously or sequentially (in any order). Suitable therapeutically effective dosages for each agent may be lowered due to the additive action or synergy.

As a non-limiting example, the methods described herein can be combined with other therapies that block inflammation (e.g., via blockage of IL1, INFα/β, IL6, TNF, IL13, IL23, etc.).

In some embodiments, the compositions and methods disclosed herein are useful to enhance the efficacy of vaccines directed to tumors or infections. Thus, the compositions and methods described herein can be administered to a subject either simultaneously with or before (e.g., 1-30 days before) a reagent (including but not limited to small molecules, antibodies, or cellular reagents) that acts to elicit an immune response (e.g., to treat cancer or an infection) is administered to the subject.

The compositions and methods described herein can be also administered in combination with an anti-tumor antibody or an antibody directed at a pathogenic antigen or allergen.

The compositions and methods described herein can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 41BB, OX40, etc.). The inhibitory treatments described herein can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e), in any of the aforementioned forms or formulations, alone or in combination with each other or other agents.

Therapeutic methods described herein can be combined with additional immunotherapies and therapies. For example, when used for treating cancer, NKT cells described herein can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors described herein include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In some embodiments, the inhibitors described herein can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

The present invention provides methods which comprise administering a pharmaceutical composition comprising any of the exemplary multispecific molecules described herein in combination with one or more additional therapeutic agents. Exemplary additional therapeutic agents that may be combined with or administered in combination with a multispecific molecule of the present invention include, e.g., an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as Her2/ErbB2, ErbB3 or ErbB4 (e.g., anti-ErbB2, anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET antagonist (e.g., an anti-cMET antibody), an IGF1R antagonist (e.g., an anti-IGF1R antibody), a B-raf inhibitor (e.g., vemurafenib, sorafenib, GDC-0879, PLX-4720), a PDGFR-a inhibitor (e.g., an anti-PDGFR-a antibody), a PDGFR-β inhibitor (e.g., an anti-PDGFR-β antibody), a VEGF antagonist (e.g., a VEGF-Trap, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354 such as REGN421), an Ang2 antagonist (e.g., an anti-Ang2 antibody disclosed in US 2011/0027286 such as H1H685P), a FOLH1 (PSMA) antagonist, a PRLR antagonist (e.g., an anti-PRLR antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin antibody), etc. Other agents that may be beneficially administered in combination with the multispecific molecules of the invention include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions of the present invention may also be administered as part of a therapeutic regimen comprising one or more therapeutic combinations selected from “ICE”: ifosfamide (e.g., Ifex®), carboplatin (e.g., Paraplatin®), etoposide (e.g., Etopophos®, Toposar®, VePesid®, VP-16); “DHAP”: dexamethasone (e.g., Decadron®), cytarabine (e.g., Cytosar-U®, cytosine arabinoside, ara-C), cisplatin (e.g., Platinol®-AQ); and “ESHAP”: etoposide (e.g., Etopophos®, Toposar®, VePesid®, VP-16), methylprednisolone (e.g., Medrol®), high-dose cytarabine, cisplatin (e.g., Platinol®-AQ).

The present invention also includes therapeutic combinations comprising any of the antigen-binding molecules mentioned herein and an inhibitor of one or more of VEGF, Ang2, DLL4, EGFR, ErbB2, ErbB3, ErbB4, EGFRVIII, cMet, IGF1R, B-raf, PDGFR-a, PDGFR-B, FOLH1 (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antibody fragment (e.g., Fab fragment; F(ab′)2 fragment; Fd fragment; Fv fragment; scFv; dAb fragment; or other engineered molecules, such as diabodies, triabodies, tetrabodies, minibodies and minimal recognition units). The multispecific molecules of the invention may also be administered and/or co-formulated in combination with antivirals, antibiotics, analgesics, corticosteroids and/or NSAIDs. The antigen-binding molecules of the invention may also be administered as part of a treatment regimen that also includes radiation treatment and/or conventional chemotherapy.

Non-limiting examples of chemotherapeutic compounds which can be used in combination treatments include, for example, aminoglutethimide, amsacrine, anastrozole, asparaginase, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

For treatment of infections, combined therapy described herein can encompass co-administering compositions and methods described herein with an antibiotic, an anti-fungal drug, an anti-viral drug, an anti-parasitic drug, an anti-protozoal drug, or a combination thereof.

Non-limiting examples of useful antibiotics include lincosamides (clindomycin); chloramphenicols; tetracyclines (such as Tetracycline, Chlortetracycline, Demeclocycline, Methacycline, Doxycycline, Minocycline); aminoglycosides (such as Gentamicin, Tobramycin, Netilmicin, Amikacin, Kanamycin, Streptomycin, Neomycin); beta-lactams (such as penicillins, cephalosporins, Imipenem, Aztreonam); vancomycins; bacitracins; macrolides (erythromycins), amphotericins; sulfonamides (such as Sulfanilamide, Sulfamethoxazole, Sulfacetamide, Sulfadiazine, Sulfisoxazole, Sulfacytine, Sulfadoxine, Mafenide, p-Aminobenzoic Acid, Trimethoprim-Sulfamethoxazole); Methenamin; Nitrofurantoin; Phenazopyridine; trimethoprim; rifampicins; metronidazoles; cefazolins; Lincomycin; Spectinomycin; mupirocins; quinolones (such as Nalidixic Acid, Cinoxacin, Norfloxacin, Ciprofloxacin, Perfloxacin, Ofloxacin, Enoxacin, Fleroxacin, Levofloxacin); novobiocins; polymixins; gramicidins; and antipseudomonals (such as Carbenicillin, Carbenicillin Indanyl, Ticarcillin, Azlocillin, Mezlocillin, Piperacillin) or any salts or variants thereof. See also Physician's Desk Reference, 59.sup.th edition, (2005), Thomson P D R, Montvale N.J.; Gennaro et al., Eds. Remington's The Science and Practice of Pharmacy, 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J. Such antibiotics can be obtained commercially, e.g., from Daiichi Sankyo, Inc. (Parsipanny, N.J.), Merck (Whitehouse Station, N.J.), Pfizer (New York, N.Y.), Glaxo Smith Kline (Research Triangle Park, N.C.), Johnson & Johnson (New Brunswick, N.J.), AstraZeneca (Wilmington, Del.), Novartis (East Hanover, N.J.), and Sanofi-Aventis (Bridgewater, N.J.). The antibiotic used will depend on the type of bacterial infection.

Non-limiting examples of useful anti-fungal agents include imidazoles (such as griseofulvin, miconazole, terbinafine, fluconazole, ketoconazole, voriconazole, and itraconizole); polyenes (such as amphotericin B and nystatin); Flucytosines; and candicidin or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Non-limiting examples of useful anti-viral drugs include interferon alpha, beta or gamma, didanosine, lamivudine, zanamavir, lopanivir, nelfinavir, efavirenz, indinavir, valacyclovir, zidovudine, amantadine, rimantidine, ribavirin, ganciclovir, foscarnet, and acyclovir or any salts or variants thereof. See also Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Non-limiting examples of useful anti-parasitic agents include chloroquine, mefloquine, quinine, primaquine, atovaquone, sulfasoxine, and pyrimethamine or any salts or variants thereof. See also Physician's Desk Reference, 59th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

Non-limiting examples of useful anti-protozoal drugs include metronidazole, diloxanide, iodoquinol, trimethoprim, sufamethoxazole, pentamidine, clindamycin, primaquine, pyrimethamine, and sulfadiazine or any salts or variants thereof. See also Physician's Desk Reference, 59th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15th edition, (2001), McGraw Hill, NY; Berkow et al., Eds. The Merck Manual of Diagnosis and Therapy, (1992), Merck Research Laboratories, Rahway N.J.

The additional therapeutically active component(s) may be administered just prior to, concurrent with, or shortly after the administration of a multispecific molecule of the present invention; (for purposes of the present disclosure, such administration regimens are considered the administration of a multispecific molecule “in combination with” an additional therapeutically active component).

The present invention includes pharmaceutical compositions in which a multispecific molecule of the present invention is co-formulated with one or more of the additional therapeutically active component(s) as described elsewhere herein.

### Administration Regimens

According to certain embodiments of the present invention, multiple doses of a multispecific molecule may be administered to a subject over a defined time course. The methods according to this aspect of the invention comprise sequentially administering to a subject multiple doses of a multispecific molecule of the invention. As used herein, “sequentially administering” means that each dose of a multispecific molecule is administered to the subject at a different point in time, e.g., on different days separated by a predetermined interval (e.g., hours, days, weeks or months). The present invention includes methods which comprise sequentially administering to the patient a single initial dose of a multispecific molecule, followed by one or more secondary doses of the multispecific molecule, and optionally followed by one or more tertiary doses of the multispecific molecule.

The terms “initial dose,” “secondary doses,” and “tertiary doses,” refer to the temporal sequence of administration of the multispecific molecule of the invention. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the multispecific molecule, but generally may differ from one another in terms of frequency of administration. In certain embodiments, however, the amount of a multispecific molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of multispecific molecule which is administered to a patient prior to the administration of the very next dose in the sequence with no intervening doses.

The methods according to this aspect of the invention may comprise administering to a patient any number of secondary and/or tertiary doses of a multispecific molecule. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples are put forth so as to provide those of ordinary skill in the art with a complete disclosure and description of how to make and use the methods and compositions of the invention, and are not intended to limit the scope of what the inventors regard as their invention. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Multispecific T-Cell Activator

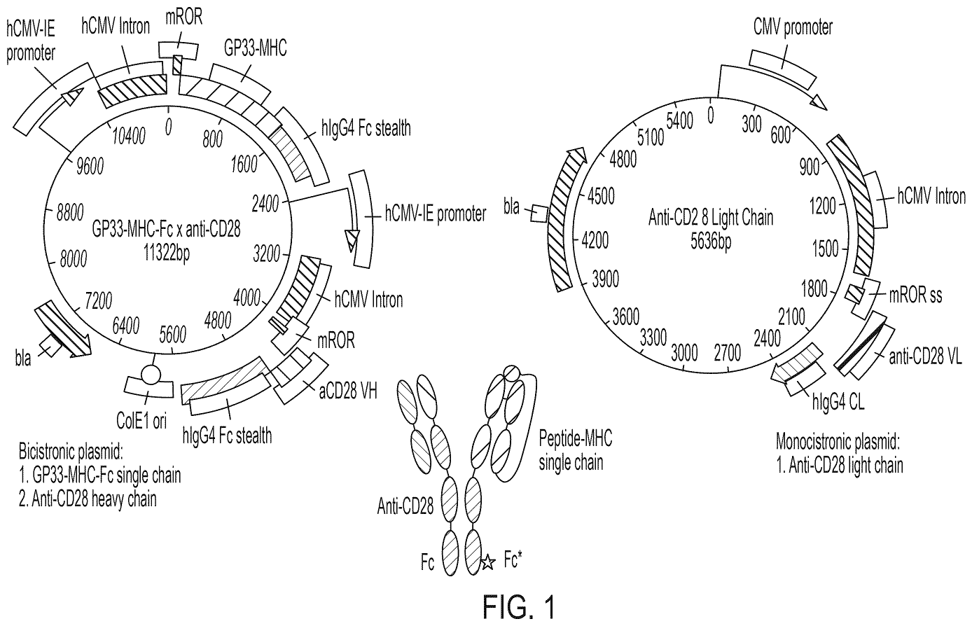

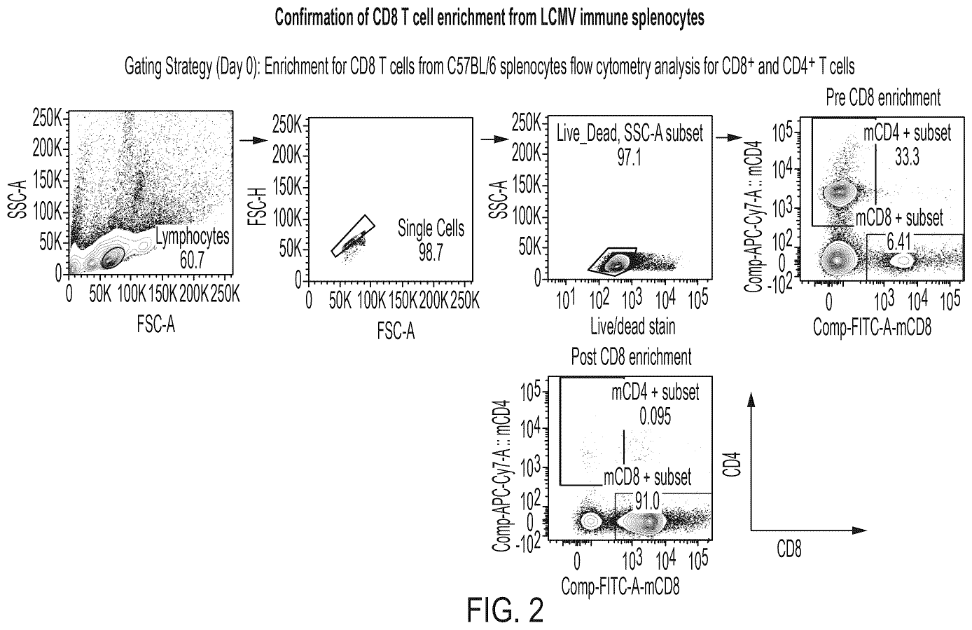

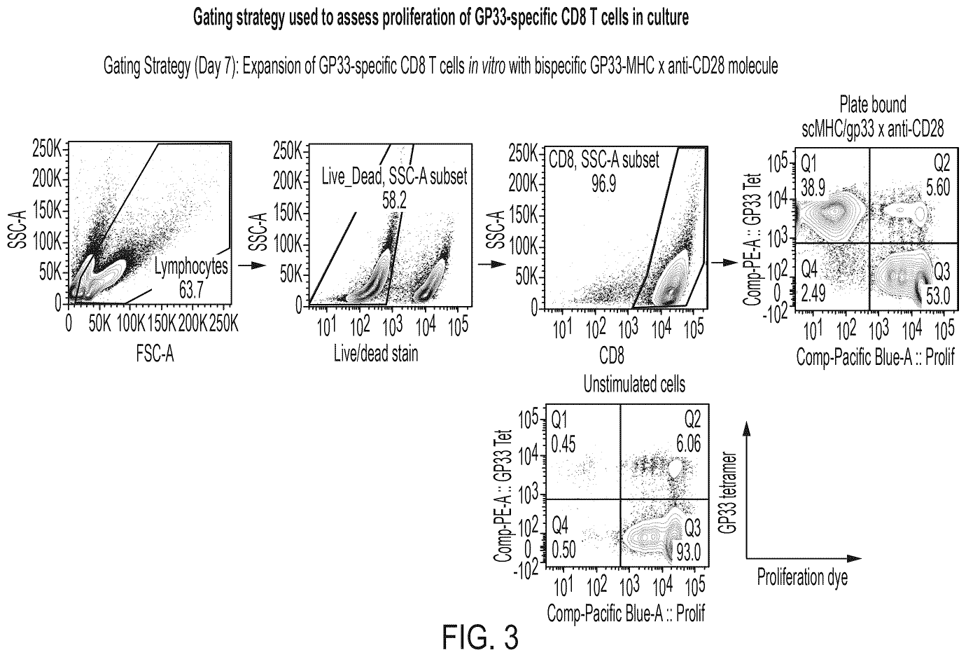

A multispecific molecule was prepared by expression of the two plasmids illustrated in FIG. 1 in a CHO cell line. The resulting structure of the multispecific molecule is also illustrated in FIG. 1. The multispecific molecule includes a first molecule comprising an anti-CD28 specific binding domain (heavy chain and light chain) and a second molecule comprising from 5′ to 3′: a lymphocytic choriomeningitis virus (LCMV) glycoprotein 33 (GP33) peptide, a Beta-2 microglobulin protein, H2-Db MHC protein and hlgG4 Fc as a single chain peptide-MHC-Fc fusion protein. In this example, the Fc region for both molecules is of the IgG4 isotype and includes a chimeric hinge. The peptide-MHC molecule further includes an Fc region comprising a modified C_{H}3 domain. The modified C_{H}3 domain was prepared with the dipeptide modification H435R/Y436F, according to EU numbering (H95R/Y96F, by IMGT exon numbering) (also known as FcΔΔdp, as described in US20100331527, which is incorporated herein by reference in its entirety for all purposes). The presence of the dipeptide modification in the heavy chain of one of the multispecific monomers but not the other facilitates differential purification of the heterodimers via differential affinity to Protein A in a chromatography platform.

Hamster anti-CD28 binding domains were recombinantly made (PV-1 clone; Abe R, et al. J. Immunol. 154:985-997, 1995). The nucleic acid and amino acid sequence identifiers are set forth in Table 2.

TABLE 2
Sequence Identifiers

| | SEQ ID NOs: | |
|---|---|---|
| | Nucleic | Amino |
| Sequence Description | Acid | Acid |
| Human IgG kappa Constant Light Chain | 1 | 2 |
| Anti-CD28 IgG4 Moiety Used in the Multispecific | 3 | 4 |
| Molecule | | |
| Anti-CD28 kappa LC | 27 | 28 |
| GP33 Peptide | 5 | 6 |
| Beta-2 Microglobulin | 7 | 8 |
| H2-Db MHC | 9 | 10 |
| GP33 PiG-IgG4 Fc Fusion Protein | 11 | 12 |

The Anti-CD28 light chain nucleic acid was cloned upstream of the IgG4 constant light chain (IgG4 CL) and downstream of a CMV promoter in one plasmid (monocistronic plasmid, FIG. 1). The two Fc-containing polynucleotides encoding 1) the peptide-MHC-Fc fusion protein and 2) anti-CD28 heavy chain, were both cloned into a second plasmid (bicistronic plasmid) as shown in FIG. 1. The GP33 nucleic acid encoding the peptide in the groove (PiG) of the MHC complex (the nine amino acid sequence KAVYNFA™, SEQ ID NO: 6; PDB: 2F74_C) is cloned downstream of a signal sequence. Peptide linkers join the PiG to the β2 microglobulin, the β2 microglobulin to the H2-Db MHC protein, and the H2-Db MHC protein to the IgG4Fc. Transcription of each Fc-containing polynucleotide is driven by its own upstream CMV promoter/intron.

### Example 2: Isolation of Antigen (GP33)-Specific Cytotoxic T-Cells

Splenocytes were isolated from mice previously infected with LCMV Armstrong (2×105 ffu i.p.; >21 days post infection). CD8+ T cells were enriched from splenocytes using negative selection with EasySep mouse CD8+ T cell isolation kit (StemCell Technologies) to remove all other (non-CD8+) T cell phenotypes. Confirmation of CD8+ T cell enrichment was conducted by flowcytometric gating on singlet, live, lymphocyte cells and stained with live/dead stain (Life Technologies), anti-mCD8a (Biolegend cat #100724), and anti-mCD4 (Biolegend cat #100428). The gating strategy for enrichment of CD8+ T cells from C57BL/6 splenocytes via flow cytometry is shown in FIG. 2. For verification of CD8 T cell enrichment, samples were acquired on a BD FACSCanto II and analyzed using FlowJo software (TreeStar). Small resting lymphocytes (gated on the SSC-A×FSC-A parameters) were further gated for singlets (FSC-H×FSC-A) and for live cells (live/dead stain negative). The live singlet cells were then plotted for mCD4×mCD8a parameters. Splenocytes enriched for CD8+ T cells show greater than 90% of cells staining positive for a CD8 marker compared to ~6% CD8+ cells in unenriched splenocytes.

The isolated CD8+ T cells were maintained in T cell Growth media (RPMI 1640, 10% FBS, 1% of 100X PSG, 2-mercaptoethanol (5 μM), Sodium pyruvate (1 mM), HEPES (20 mM), IL-2 at 8 ng/μl, IL-7 delivered at 10 ng/ml) for the proliferation experiments discussed in the examples below.

### Example 3: Expansion of Antigen (GP33)-Experienced Cytotoxic T-Cells

Cell culture conditions: CD8+ T cells were labeled with CellTrace Violet Cell Proliferation Kit (Invitrogen) and cultured in 24 well plates at a final concentration of 2×106 cells/ml in 0.5 ml media. Where indicated in Example 4, below, IL-7 (10 μg/ml) and IL-2 (8 ng/μl) cytokines were added to cultures at day 1 and day 4, respectively.

The gating strategy for assessment of the proliferation of the GP33-specific CD8+ T cells (isolated in Example 2) in culture is shown in FIG. 3. The T cells were contacted with a plate-bound version of the multispecific molecule of Example 1, and the corresponding proliferation was compared to an unstimulated control (cultured T cells in the absence of the multispecific molecule). Note that the CellTrace dye intensity decreased by 50% with each cell division, and the degree of stimulation/activation is gauged by the extent of loss of the proliferation dye. Cells were stained with CellTrace and placed into culture under various stimulation conditions and allowed to grow for 7 days. On day 7 cells were removed and stained with live/dead stain, anti-mCD8a (Biolegend cat #100724), and anti-gp33 tetramer. Samples were acquired on BD FACSCANTO II and analyzed using FlowJo software. Cells were gated using SSC-A×FSC-A parameters, live/dead stain negative cells, and mCD8a positive cells and then analyzed for GP33 tetramer×Proliferation dye (CellTrace) parameters. Unstimulated cells maintained bright CellTrace staining post culture (right panel) while those stimulated with scMHC/GP33× anti-mCD28 multispecific demonstrated a significant decrease in CellTrace brightness in the tetramer positive staining cells (~39% tetramer+ Proliferation dye dim) indicating a greater degree of cell division.

### Example 4: Effect of Plate-Bound Multispecific GP33-MHC×Anti-CD28 on the Expansion of Antigen (GP33)-Specific CD8+ T Cells Stimulated with and without Cytokines

In this example, the effect of cytokines (IL-2 and IL-7) in culture on the expansion of GP33-specific CD8+ T cells stimulated in vitro was examined with plate bound multispecific GP33-MHC×anti-CD28 (see Example 1). The multispecific molecule of Example 1 was prepared at 30 nM (~5 μg/ml) in PBS solution and 300 μl of the solution was added to each wells of a 24-well culture plate. The plate was sealed and incubated for 2 hrs at 37° C. or overnight at 4° C. The solution was removed just prior to the addition of 5×10⁵ cells in 0.5 ml media. As noted above, the CellTrace dye intensity decreased by 50% with each cell division, and the degree of stimulation/activation was gauged by the extent of loss of the proliferation dye.

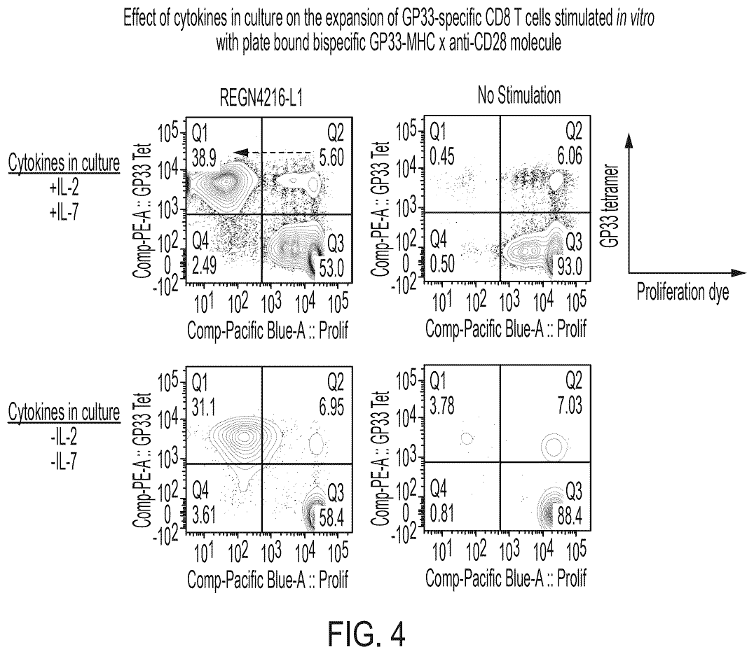

As shown in FIG. 4, CD8+ T cells cultured with the multispecific molecule demonstrated maximal proliferation of GP33 specific T cells, which does not occur in unstimulated cultures. Note that in FIG. 4, the number of cells specific for the antigen (GP33) increased to almost 39% (see Quadrant 1 (Q1)) in the population of cells stimulated with plate-bound multispecific molecule and maintained in culture with cytokines. Almost no antigen-specific T cells were observed in the population without plate-bound multispecific molecule, despite the presence of cytokines. Although cytokines in the cell culture supported the survival of T cells (compare both no stimulation panels), the proliferation of GP33 specific T cells was dependent on multispecific stimulation and not attributed to cytokines alone.

### Example 5: Effect of Titration of a Polyclonal Antibody Cross-linker Relative to Multispecific GP33-MHC×anti-CD28 on the Expansion of Antigen (GP33)-Specific CD8+ T Cells in vitro

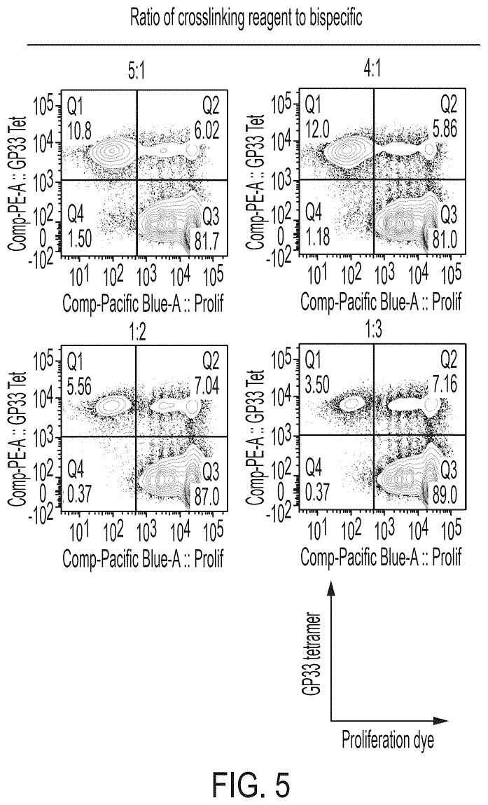

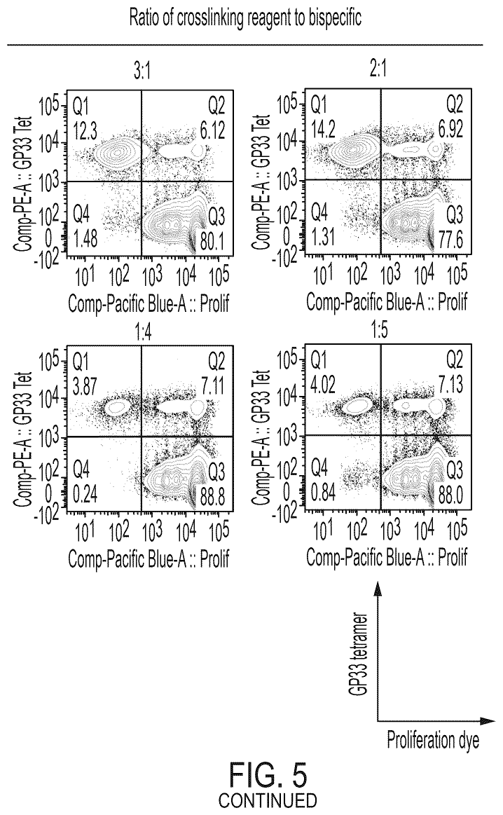

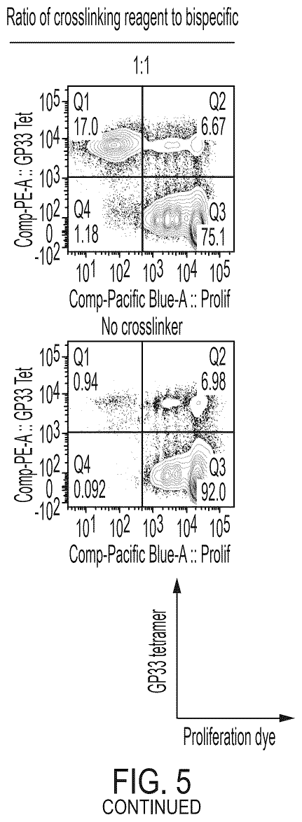

In this example, the effect of titration of a polyclonal antibody cross-linker (Thermo Scientific Pierce, Goat anti-Human IgG (H+L), Cross-Adsorbed Secondary Antibody, Prod #31119, 1.8 mg/ml (MW~144 kDa)) relative to the multispecific GP33-MHC×anti-CD28 (see Example 1) was examined on the expansion of GP33-specific CD8+ T cells in vitro. The cross-linking polyclonal Ab was pre-complexed with the multispecific molecule at the ratios shown in FIG. 5 prior to addition to the CD8+ T cells. Briefly, the goat polyclonal anti-human IgG (H+L) and multispecific molecule were combined at molar ratios ranging from 5:1 to 1:5 (150 nM: 30 nM to 30 nM: 150 nM) pAb/multispecific molecule in 0.25 ml media in tubes. The mixtures were incubated for 15 min on ice and then added to cells in 0.25 ml and incubated for additional 15 min on ice before being placed in a 24 well plate at 37° C. Cytokines (IL-2 and IL-7) were used in the culture of the cells. As noted above, the CellTrace dye intensity decreased by 50% with each cell division, and the degree of stimulation/activation was gauged by the extent of loss of the proliferation dye.

Cells were cultured 7 days with complexes of pAb and multispecific molecules formed with varying ratios of crosslinking pAb to multispecific and then analyzed for proliferation as described in Example 3. Cells cultured with complexes derived from ratios of crosslinker to multispecific of greater than 1 in general demonstrated better stimulation of GP33 specific cells than ratios where crosslinker was less than multispecific. The 1:1 ratio of polyclonal Ab to multispecific molecule (30 nM: 30 nM) appears to provide efficient crosslinking and therefore efficient proliferation of antigen-specific T cells (Q1=17.9%). All tested ratios of crosslinker to multispecific molecule (5:1-1:5) demonstrated significant Ag-specific T cell proliferation compared to control well containing multispecific molecules without any crosslinker (bottom right panel), as shown in Table 3, below.

TABLE 3
Percentage of Antigen-Specific Proliferating T Cells as a Function
of the Ratio of Crosslinking Reagents to Multispecific Molecules

| % of Antigen-Specific | Ratio of Crosslinking |
|---|---|
| Proliferating T Cells | Reagent:Multispecific Molecule |
| 10.8 | 5:1 |
| 12.0 | 4:1 |
| 12.3 | 3:1 |
| 14.2 | 2:1 |
| 17.0 | 1:1 |
| 5.56 | 1:2 |
| 3.50 | 1:3 |
| 3.87 | 1:4 |
| 4.02 | 1:5 |
| 0.94 | No crosslinker |

### Example 6: Stimulation of Antigen-Specific CD8+ T Cells with Multispecific GP33-MHC×Anti-CD28 with IgG1 Fc Arrayed on Human Embryonic Kidney (HEK293-hFcR1) Cells

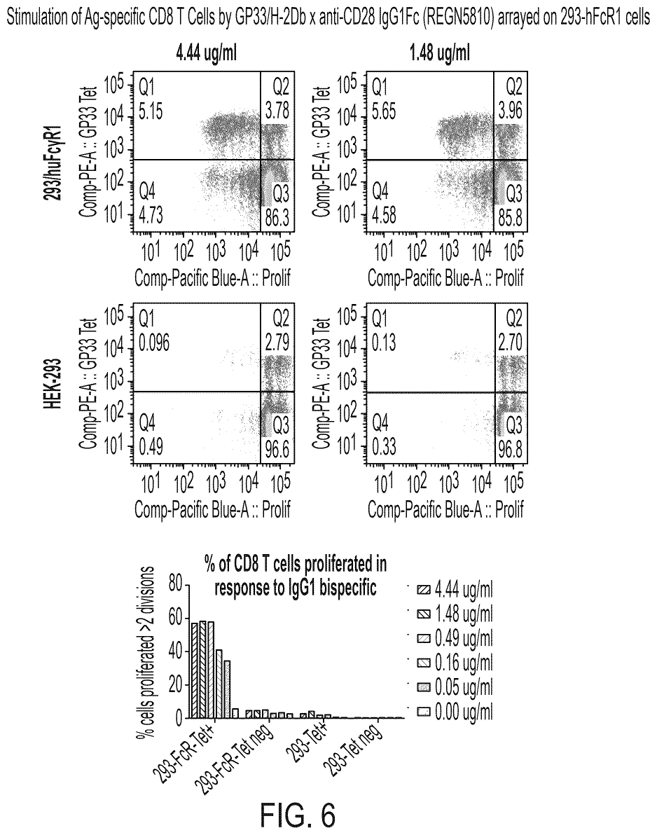

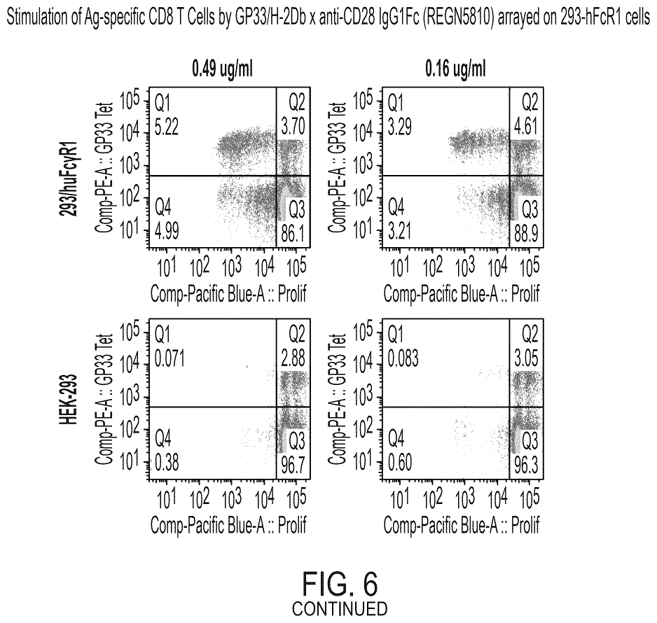

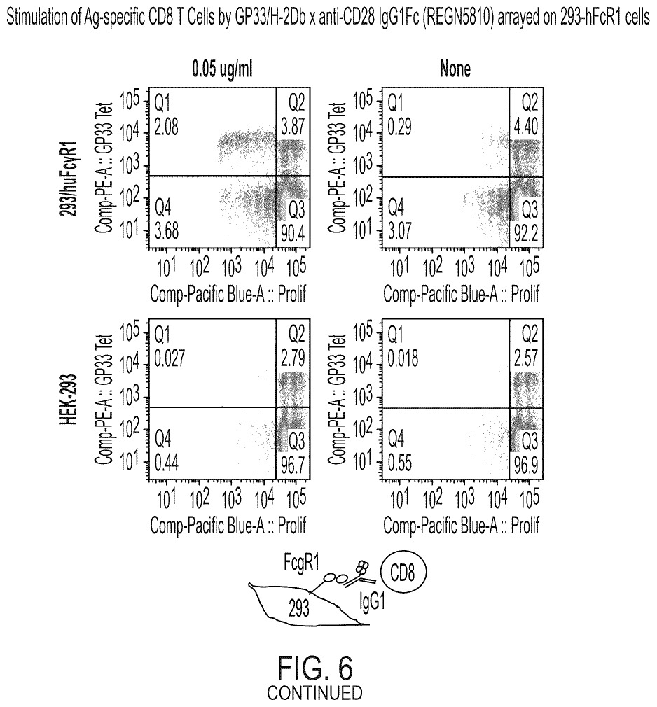

In this example, the stimulation of antigen-specific CD8+ T cells was measured, in which the multispecific GP33-MHC×anti-CD28 with IgG1 Fc was added to a co-culture of CD8+ T cells from an LCMV immune mouse with HEK293 cells expressing hFcgR1 (or alternatively parental HEK293 control cells not expressing hFcgR1). Briefly, the HEK293 cells were pretreated with 50 μg/ml mitomycin C for 1 hr at 37° C. and washed twice with PBS prior to co-culture with T cells. Both cell types were resuspended in T cell culture media and combined in a 96-well round-bottom culture plate at 1:1 cell ratio in 0.2 ml media. Multispecific GP33-MHC×anti-CD28 IgG1 reagent was added to the co-culture at concentrations indicated in FIG. 6 and incubated at 37° C. overnight. Cultures were expanded to 0.5 ml media in a 24-well plate on day+1 with addition of IL-2 (8 ng/ml) and IL-7 (10 ng/ml). Cultures were grown for 4 days and assessed for proliferation as above. As shown in FIG. 6, robust gp33-specific T cell expansion was only observed when the IgG1 Fc multispecific molecule was co-cultured with 293 cells expressing the hFcgR1, and not with parental 293 cells. Furthermore, this effect was titratable with higher concentrations of the multispecific molecule mediating more robust T cell proliferation, while no proliferation of T cells was observed in the absence of the multispecific molecule reagent.

The nucleic acid and amino acid sequence identifiers are set forth in Table 4.

TABLE 4
Sequence Identifiers

| | SEQ ID NOs: | |
|---|---|---|
| | Nucleic | Amino |
| Sequence Description | Acid | Acid |
| Human IgG kappa Constant Light Chain | 1 | 2 |
| Anti-CD28 IgG1 Moiety Used in the Multispecific | 13 | 14 |
| Molecule | | |
| GP33 PiG-IgG1 Fc Fusion Protein | 15 | 16 |

### Example 7: Stimulation of Antigen-Specific CD8+ T-cells with Multispecific GP33-MHC×Anti-CD28 with IgG4/2-stealth Fc Arrayed on Human Embryonic Kidney (HEK293-anti-hFc scFv) Cells

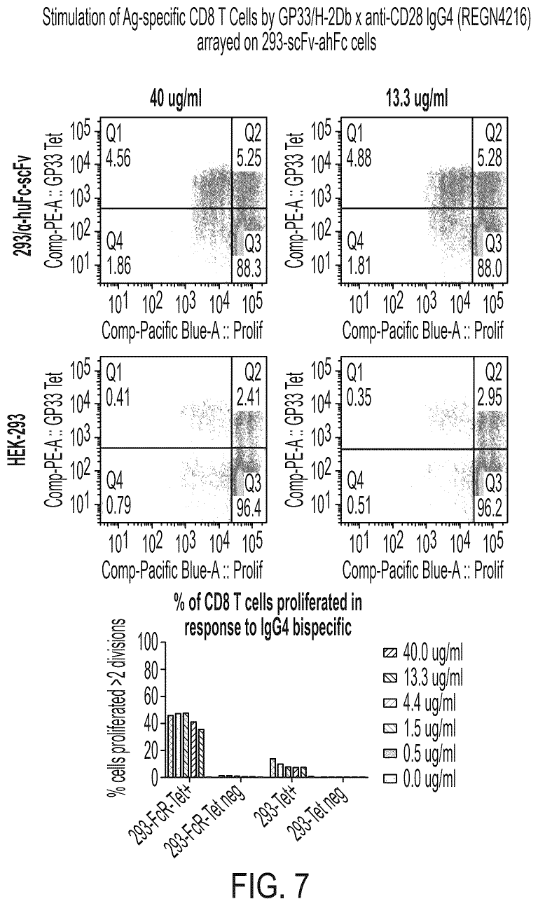

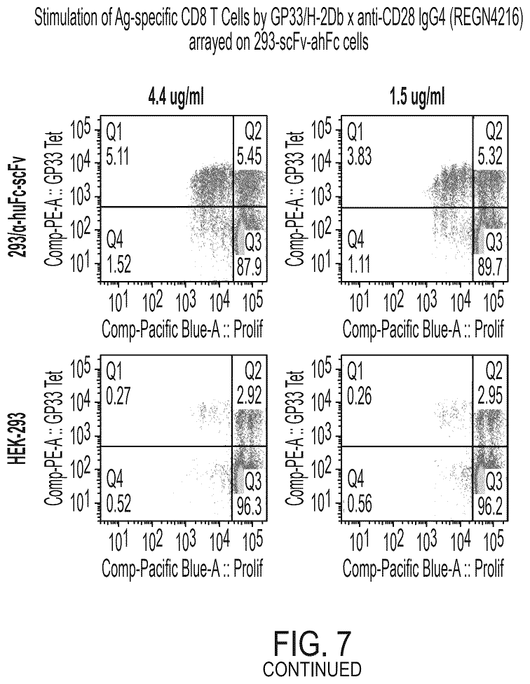

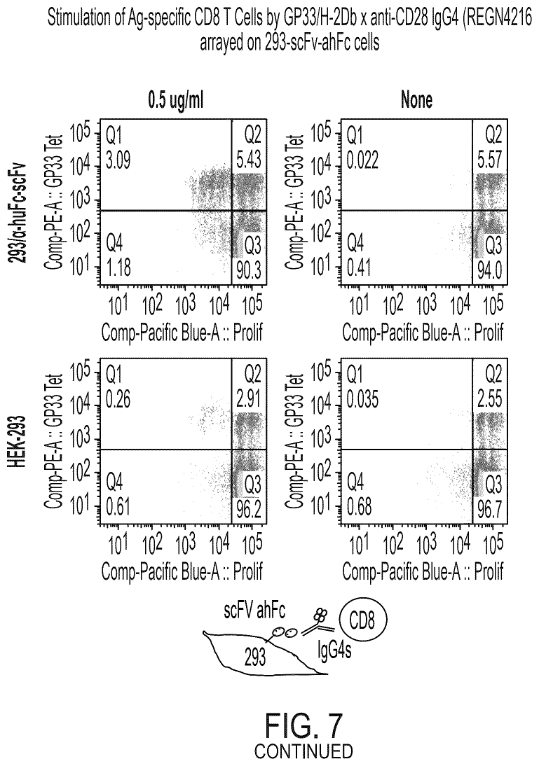

In this example, using the method described in Example 6, the stimulation of antigen-specific CD8+ T cells was measured, in which the multispecific GP33-MHC×anti-CD28 with IgG4 Fc (Example 1) was bound to HEK293 cells via binding of the IgG4 Fc to an anti-human Fc scFv expressed on the HEK293 cells. As shown in FIG. 7, robust gp33-specific T cell expansion was only observed when the IgG4 Fc multispecific molecule was co-cultured with 293 cells expressing the anti-hFc scFv, and not with parental 293 cells. This effect was titratable with higher concentrations of the multispecific molecule mediating more robust T cell proliferation, while no proliferation of T cells was observed in absence of the multispecific molecule reagent.

The nucleic acid and amino acid sequence identifiers are set forth in Table 5.

TABLE 5
Sequence Identifiers

| | SEQ ID NOs: | |
|---|---|---|
| | Nucleic | Amino |
| Sequence Description | Acid | Acid |
| Anti-CD28 IgG4 Moiety Used in the Multispecific | 3 | 4 |
| Molecule | | |
| GP33 PiG-IgG4 Fc Fusion Protein | 11 | 12 |

### Example 8: Stimulation of Antigen-Specific CD8+ T Cells with Multispecific GP33-MHC×Anti-CD28 with IgG4/2-stealth Fc and C-terminal anti-CD20 scFv Arrayed on Cells

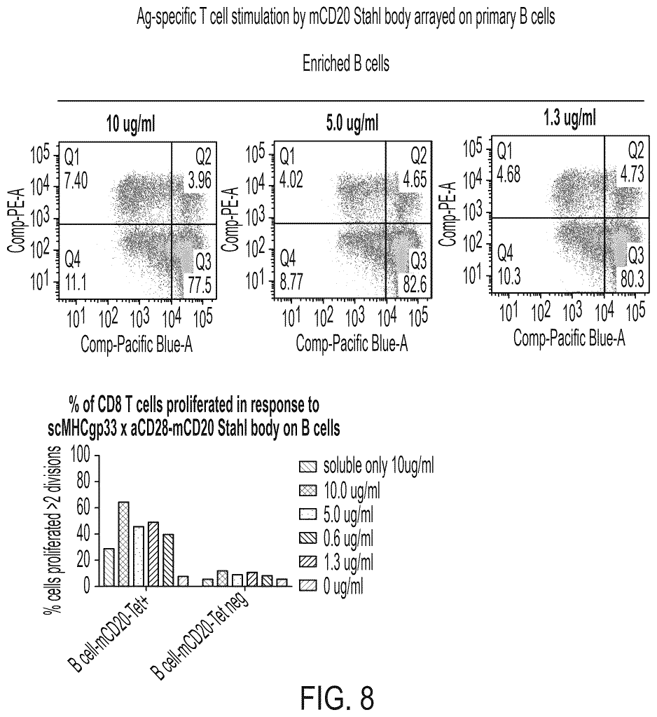

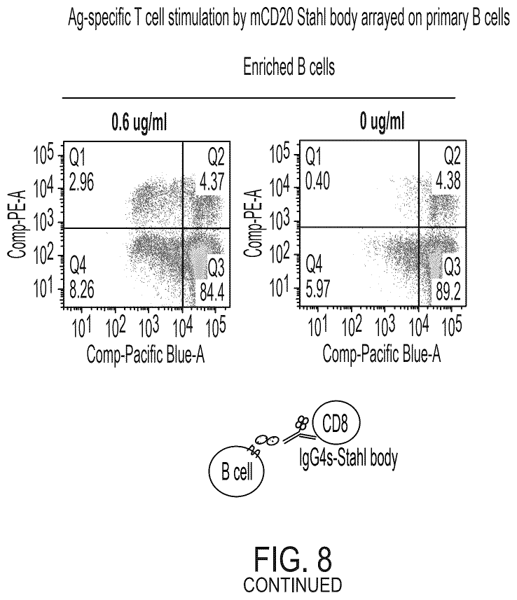

In this example, the stimulation of antigen-specific CD8+ T cells was measured, in which the multispecific GP33-MHC×anti-CD28 with IgG4 Fc (see Example 1) further included a C-terminal anti-CD20 scFv (aka mCD20 Stahl body) to array the multispecific molecules on primary B cells via CD20 binding. Primary mouse B cells were enriched from mouse splenocytes using an immunomagnetic negative selection protocol (EasySep™ Mouse B Cell Isolation Kit; Stem Cell Technologies). Similar to methods in prior examples, the mCD20 Stahl body was added at varying concentrations to wells containing CD8+ T cells from LCMV immune mice and B cells enriched from a naïve mouse spleen. As shown in FIG. 8, robust gp33-specific T cell expansion was observed when the mCD20 Stahl body was co-cultured with primary B cells. This effect was titratable with higher concentrations of the multispecific molecule mediating more robust T cell proliferation, while no proliferation of T cells was observed in absence of the multispecific molecule reagent. Human CD20 and human CD19 scFv Stahl body variants have also been tested and show similar results.

The nucleic acid and amino acid sequence identifiers are set forth in Table 6.

TABLE 6
Sequence Identifiers

| | SEQ ID NOs: | |
|---|---|---|
| Sequence Description | Nucleic Acid | Amino Acid |
| anti-CD20 scFv | 17 | 18 |
| GP33 PiG- IgG4/2-stealth Fc - anti-CD20 | 19 | 20 |

### Example 9: Inducing T Cell Divisions with Plate-Bound or Cell-Bound Multispecific GP33-MHC×Anti-CD28 Having a C-Terminal scFv for Binding to a Cell-Surface Molecule

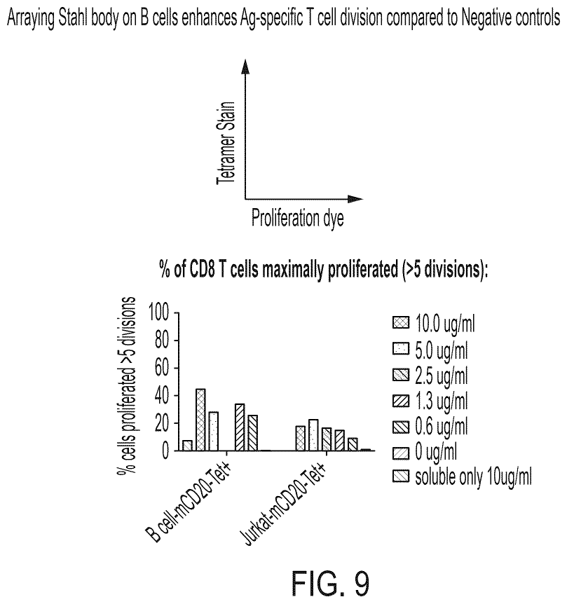

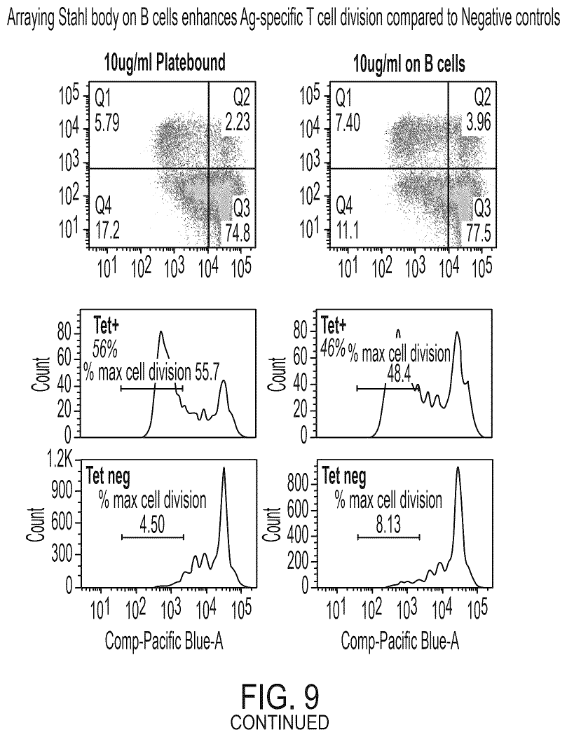

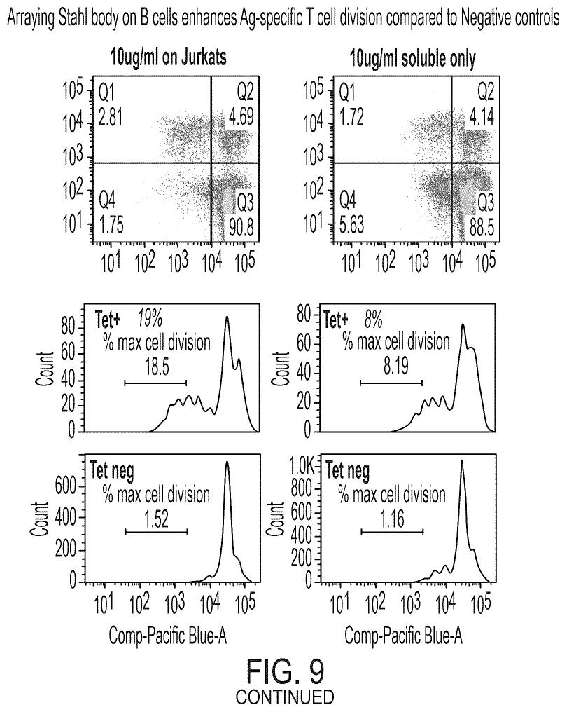

In this example, the induction of antigen-specific CD8+ T cell divisions was measured, in which the multispecific GP33-MHC×anti-CD28 with IgG4 Fc (Example 1) and further including a C-terminal anti-CD20 scFv (herein referred to as Stahl body) was added to T cell cultures under four conditions: 1) plate-bound reagent, 2) soluble reagent, 3) soluble reagent with co-cultured primary B cells, or 4) soluble reagent with co-cultured Jurkat cells. The flowcytometry plots and histogram analysis in FIG. 9 demonstrate that using B cells to present the mCD20 Stahl body to the T cells results in maximal proliferation of more Ag-specific T cells compared to cultures of Stahl body with T cells only, as well as cultures of Stahl body with T cells and CD20 negative Jurkat cells. The maximal proliferation of T cells from the Stahl body/B cell co-culture resembled the proliferation profiles that were observed with the plate-bound reagent positive control.

### Example 10: Stimulation of Antigen-Specific CD8+ T Cells with a Virus-Like Particle (VLP) Arrayed with scGP33-MHC and Anti-CD28

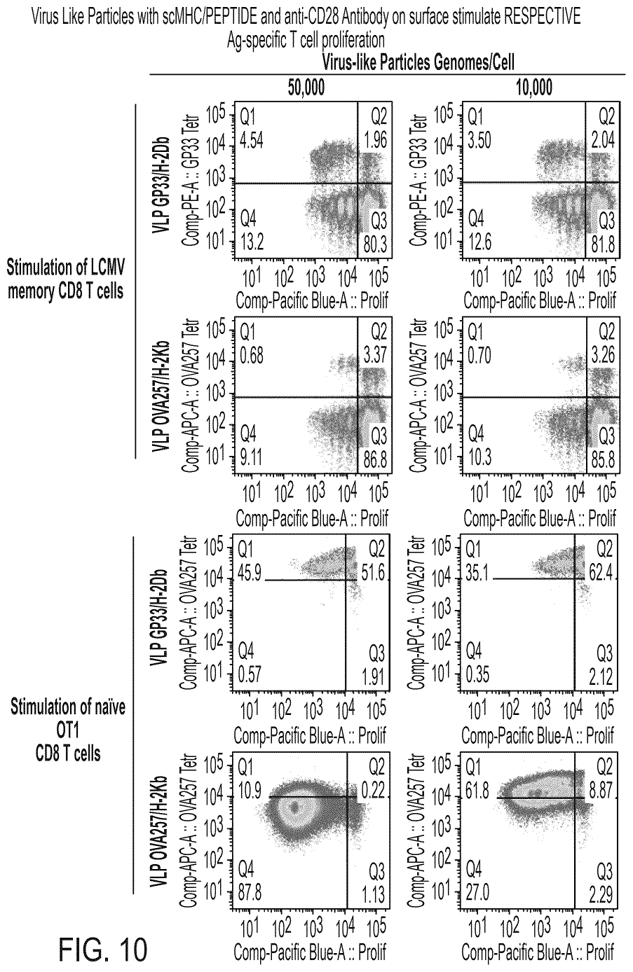

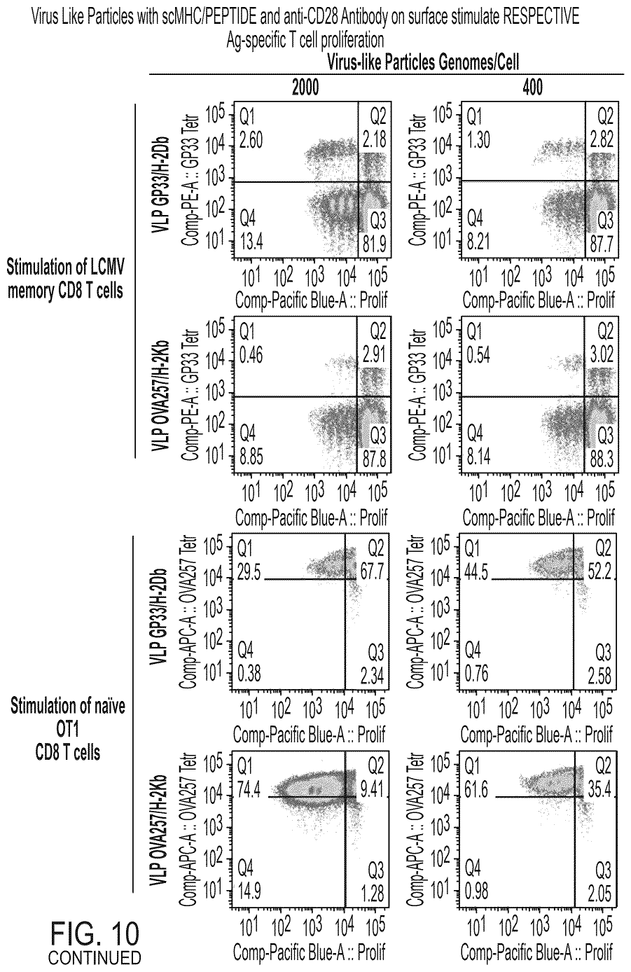

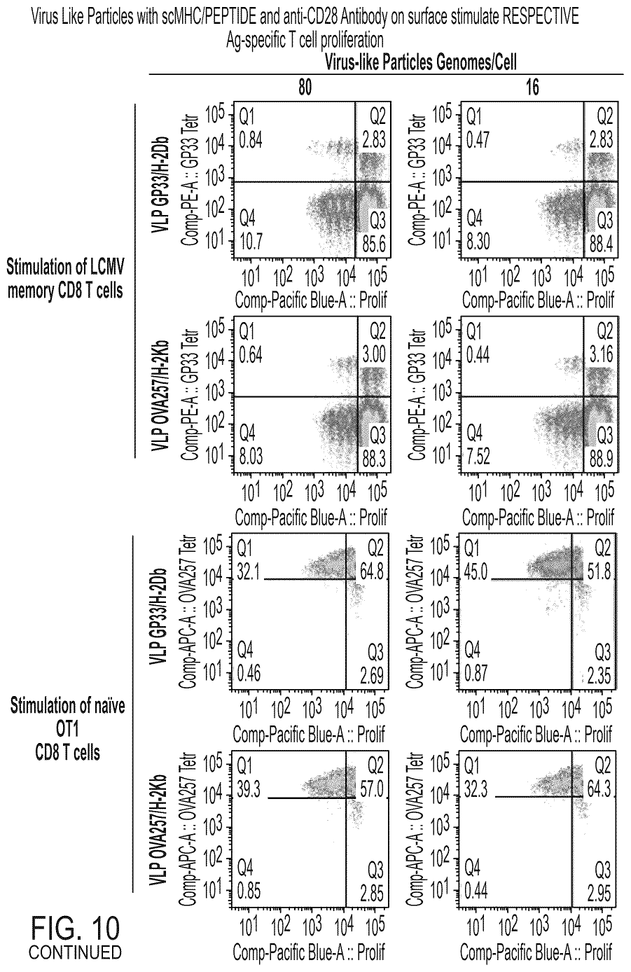

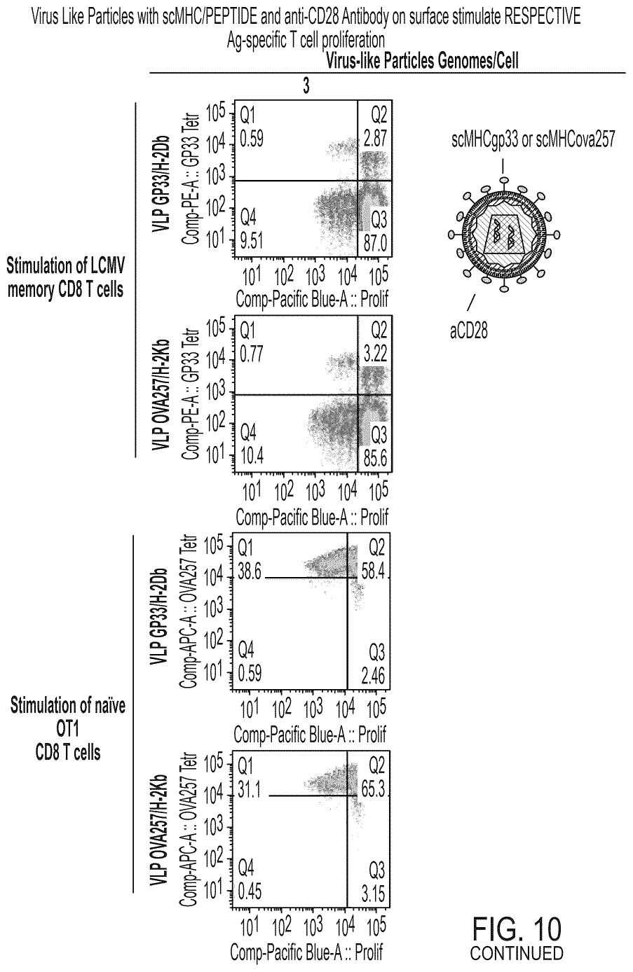

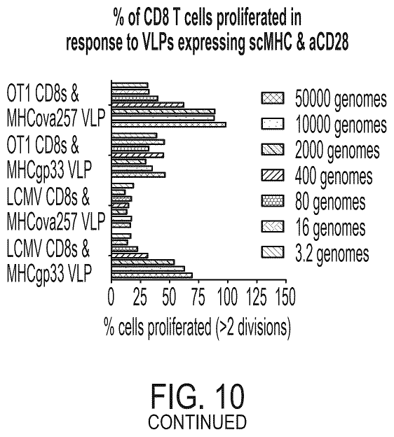

In this example, the stimulation of antigen-specific CD8+ T cells was measured using a virus-like particle arrayed with single chain GP33-MHC and anti-CD28. VLPs were produced with scMHCgp33 or scMHCova257 on the surface in combination with a membrane version of anti-mCD28 antibody (clone PV-1). Briefly, 293T cells were transfected with packaging plasmid psPAX2 and expression constructs for transmembrane scMHCp, transmembrane anti-mCD28 HC, and anti-CD28 LC. VLPs were harvested from supernatants and concentrated using ultracentrifugation with a 20% sucrose cushion. VLP pellets were rehydrated in 40 μl PBS overnight at 4° C., aliquoted and stored at −80° C. VLP concentrations were assessed using Lenti-XTM qRT-PCR Titration Kit (Takara, Catalog No. 631235). As shown in FIG. 10, CD8+ T cells from either LCMV immune mice or OT1 mice (specific for ova257 epitope) were cultured with the indicated titrations of VLPs for 4 days and assessed for proliferation. scMHCgp33 VLPs specifically stimulated gp33 T cells from LCMV immune mice to proliferate, while scMHCova257 VLPs specifically stimulated OT1 CD8 T cells to proliferate.

### Example 11: Activation and Proliferation of Antigen-Specific T Cells Using B Cells Presenting Single Chain GP33-MHC or Single Chain OVA-MHC

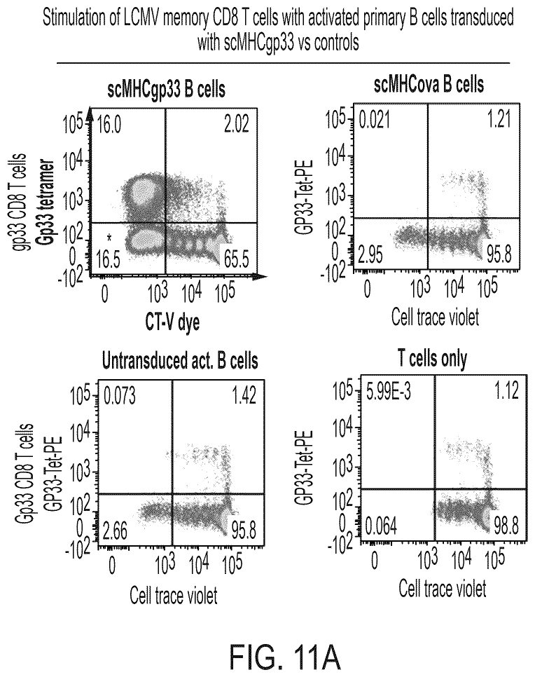

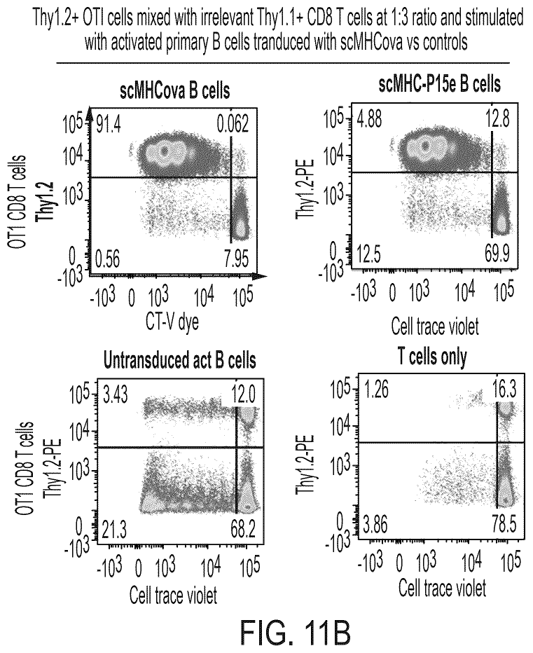

In this example, the stimulation of antigen-specific CD8+ T cells was measured, in which the single chain GP33-MHC or single chain ovalbumin (OVA) peptide-MHC including a C-terminal anti-CD20 scFv (aka mCD20 Stahl body) to array the multispecific molecules on primary B cells via CD20 binding. Primary mouse B cells were enriched from mouse splenocytes using an immunomagnetic negative selection protocol (EasySep™ Mouse B Cell Isolation Kit; Stem Cell Technologies). B cells were cultured with 50 g/ml LPS for 48 hours and then transduced with retrovirus (RV) using 5×10⁴ RV genomes/cell by spinoculation method and incubated for 24 hours prior to coculture experiment. As shown in FIG. 11A (1st panel), scMHCgp33 B cells co-cultured with CD8+ T cells from LCMV immune mice causes specific outgrowth of gp33 tetramer positive cells compared to scMHCova B cell (FIG. 11A (2^{nd} panel)), untransduced activated B cell and T cell only controls (FIG. 11A (3^{rd} and 4^{th} panel, respectively)). The large number of tetramer negative divided T cells observed with specific scMHCova B cell stimulation can be attributed to downregulation of the Ag-specific TCR due to continuous stimulation during culture. FIG. 11B provides further evidence of the in vitro antigen specificity of the engineered B cells was observed using OTI CD8+ T cells (T cells obtained from transgenic homozygous mice contain inserts for mouse Tera-V2 and Tcrb-V5 genes, wherein the transgenic T cell receptor was designed to recognize ovalbumin residues 257-264 when in the context of the MHC-Kb). scMHCova or irrelevant control scMHC-P15E RV transduced B cells (5×10⁴ RV genomes/cell) were co-cultured with a defined mixture of OTI Thy 1.2+ CD8 T cells and naïve Thy 1.1+ congenic B cells (1:3 ratio). Staining for Thy 1.2 confirms that nearly 100% of dividing cells in response to scMHCova B cells are OTI cells. T cells co-cultured with scMHC-P15e B cells or untransduced B cells resembled T cell only controls.

The nucleic acid and amino acid sequence identifiers are set forth in Table 7.

TABLE 7
Sequence Identifiers

| | SEQ ID NOs: | |
|---|---|---|
| Sequence Description | Nucleic Acid | Amino Acid |
| scMHCova | 21 | 22 |
| OVA PiG-Fc -anti-CD20 Fusion Protein | 23 | 24 |
| scMHC-P15E | 25 | 26 |

### Example 12: In Vivo Delivery of scMHCova B Cells and not Irrelevant scMHCgp33 B Cells Specifically Stimulate Proliferation of OTI CD8⁺ T Cells

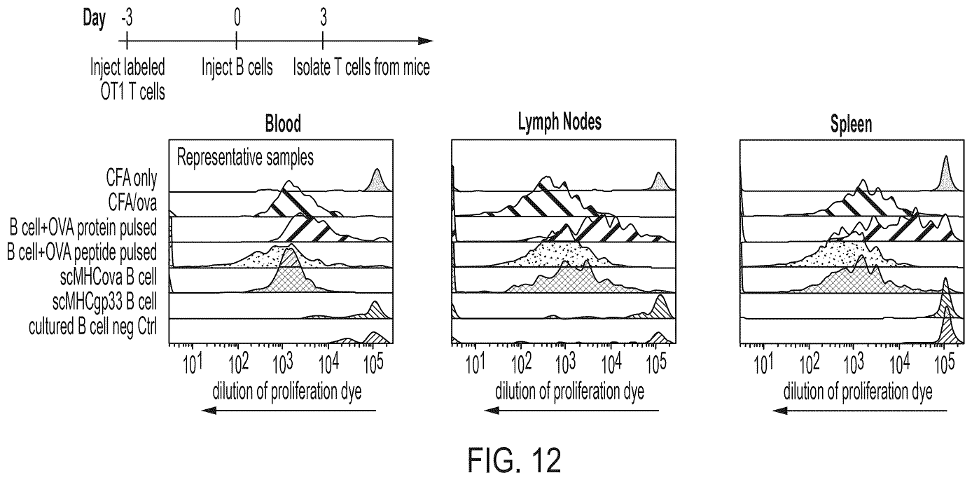

In this example, primary mouse B cells were enriched from naïve mouse splenocytes as described in previous examples and then cultured with 50 μg/ml LPS for 48 hours. B cells were treated ex vivo with the following conditions to generate cells displaying specific antigen peptides: ova protein pulsing, ova257-264 peptide pulsing, scMHCova retrovirus (RV) transduction, scMHCgp33 RV transduction, and control untransduced activated B cells. RV transduced B cells were infected with 5.5×10⁴ RV genomes/cell by spinoculation method and incubated for 24 hours before delivery to mice. B cells were incubated for 1 hour with ova protein (10 μM) or with ova257 peptide (5 μg/ml) were washed 3× before delivery to mice. The various B cells were then injected into mice (2.5×10⁶ cells/mouse) that had previously received OTI CD8+ T cells labeled with CellTrace proliferation dye (2.5×10⁶). CD8+ T cells from blood, lymph nodes, and spleen were harvested 3 days post control stimulation/B cell transfer and assessed for proliferation of OTI CD8 T cells via flowcytometry similar to previous examples. The OTI CD8⁺ T cells in mice that were injected with positive control Complete Freund's Adjuvant (CFA) with ova peptide (200 μg peptide/CFA emulsion injected SC) demonstrated robust proliferation in all compartments compared to CFA only control (FIG. 12). B cells expressing scMHCova, but not control scMHCgp33, demonstrated similar robust proliferation indicating that in vivo delivery of modified B cells can specifically stimulate OTI cells (FIG. 12).

### Example 13: In Vivo Delivery of scMHCova B Cells and VLPs Induce OTI CD8⁺ T Cells to Become Lytic Effector Cells

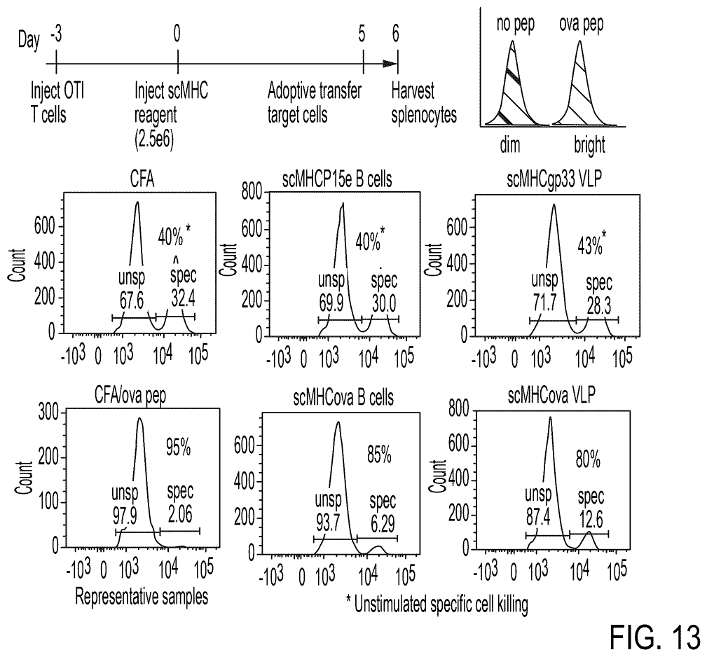

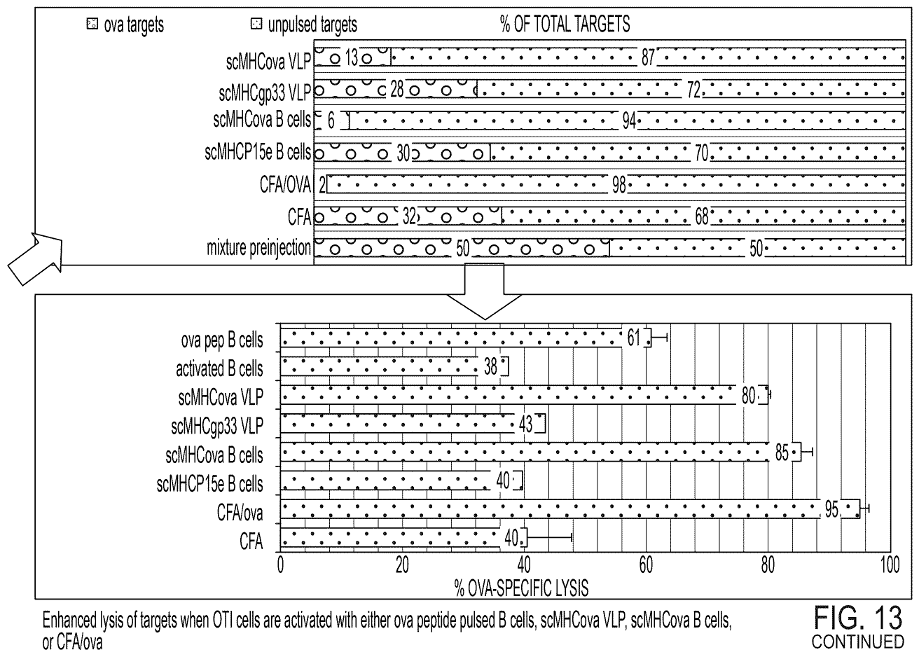

In this example, primary B cells from naïve mice were treated ex vivo as described in Example 12 with the following conditions to generate cells displaying antigen peptides: ova257 peptide pulsing, scMHCova RV transduction, scMHC-PV15e RV transduction, and control untransduced activated B cells. Modified B cells (2.5×10⁶), scMHCp VLPs (1×10⁴ genomes/OTI cell transferred), and control CFA/ova (200 μg pep/CFA) were injected into mice which had previously received 2.5×10⁶ adoptive transferred OTI CD8⁺ T cells. Target cells for lysis assay consisted of peptide-pulsed dye-labeled naïve splenocytes. Five days post treatment, 10×10⁶ target cells consisting of a 1:1 ratio of ova peptide-pulsed cells and unpulsed cells were injected IV into mice. The target cell groups were labeled with two different concentrations of CellTrace dye to be able to distinguish the respective target cell groups from each other during analysis. 20 hrs post transfer, spleens from treated mice were harvested and populations of target cells were assessed via flowcytometry. The CellTrace “bright” Ova target cells were dramatically reduced compared to the CellTrace “dim” unpulsed targets in mice treated with positive CFA/ova257 control, scMHCova B cell, and scMHCova VLP treatments relative to negative control stimulations (FIG. 13).

### Example 14: In Vivo Delivery of scMHCova Stahl Bodies and not Irrelevant scMHCgp33 Stahl Bodies Specifically Stimulate Proliferation of OTI CD8 T Cells

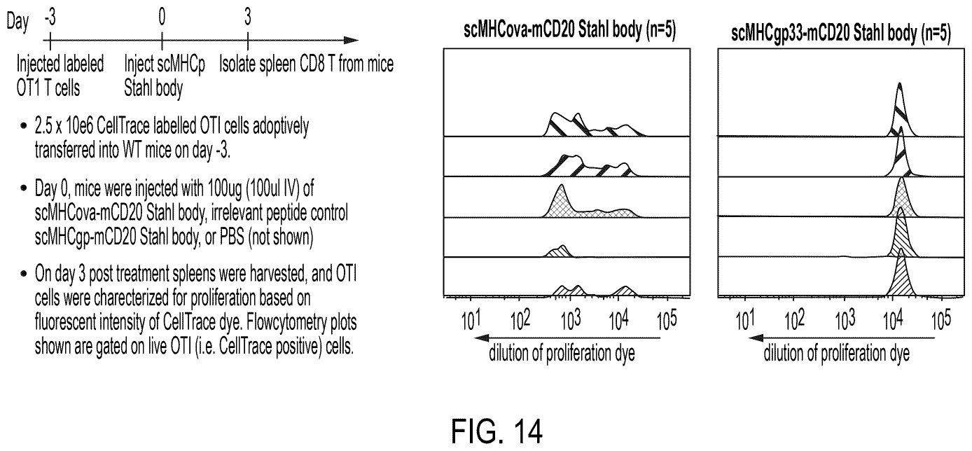

In this example, mice were adoptively transferred (IV) with 2.5×10⁶ CellTrace proliferation dye labeled OTI cells and allowed to rest for 3 days. These mice were then treated with either scMHCova-mCD20 Stahl body (100 μg in 100 μl IV), irrelevant control scMHCgp33-mCD20 Stahl body (100 μg in 100 μl IV), or phosphate buffered saline (100 μl IV). Three days post treatment the spleens were harvested from the mice and OTI cell populations were assessed for proliferation via flowcytometry. OTI cells in mice that had received scMHCova-mCD20 Stahl body showed evidence of proliferation (dilution of proliferation dye) compared to irrelevant scMHCgp33-mCD20 Stahl body (shown) or PBS control (not shown) (FIG. 14). These results indicate that in vivo delivery of a cognate scMHCp-mCD20 Stahl body can stimulate Ag-specific CD8 T cells.

Sequence Listing

| SEQ ID | | |
|---|---|---|
| NO | Description | Sequence |
| 1 | Human IgG | CGAACTGTGGCTGCACCATCTGTCTTCATCTTCCCGCCATCTGA |
| | kappa | TGAGCAGTTGAAATCTGGAACTGCCTCTGTTGTGTGCCTGCTGA |
| | Constant LC | ATAACTTCTATCCCAGAGAGGCCAAAGTACAGTGGAAGGTGGA |
| | Nucleic Acid | TAACGCCCTCCAATCGGGTAACTCCCAGGAGAGTGTCACAGAG |
| | Sequence | CAGGACAGCAAGGACAGCACCTACAGCCTCAGCAGCACCCTGA |
| | | CGCTGAGCAAAGCAGACTACGAGAAACACAAAGTCTACGCCTG |
| | | CGAAGTCACCCATCAGGGCCTGAGCTCGCCCGTCACAAAGAGC |
| | | TTCAACAGGGGAGAGTGTTAG |
| 2 | Human IgG | RTVAAPSVFIFPPSDEQLKSGTASVVCLLNNFYPREAKVQWKVDN |
| | kappa | ALQSGNSQESVTEQDSKDSTYSLSSTLTLSKADYEKHKVYACEVT |
| | Constant LC | HQGLSSPVTKSFNRGEC* |
| | Amino Acid | |
| | Sequence | |
| 3 | Anti-CD28 | ATGCACAGACCTAGACGTCGTGGAACTCGTCCACCTCCACTGG |
| | IgG4 Moiety | CACTGCTCGCTGCTCTCCTCCTGGCTGCACGTGGTGCTGATGCA |
| | Used in the | CAGGTACAATTGAAACAAAGTGGCGCCGAACTGGTAAAACCTG |
| | Multispecific | GAGCAAGTGTTAAGATCAGCTGTAAAACATCAGGATATACATT |
| | Molecule | TACCGACGGATATATGAATTGGGTGGAACAAAAACCTGGACAG |
| | Nucleic Acid | GGGCTCGAATGGATTGGAAGGATTGACCCAGATAGCGGTAACA |
| | Sequence | CAAGATACAACCAGAAATTTCAAGGCAAAGCAACTCTTACACG |
| | (underline | CGATAAATCATCATCAACTGTCTACATGGACCTGCGTTCCCTTA |
| | portion is | CTTCCGAGGATTCAGCTGTTTATTATTGCGCACGGGACGGAAC |
| | mROR signal | ATTTTATGGAACATACGGTTATTGGTATTTCGACTTCTGGGGCC |
| | sequence) | AAGGAACTCAAGTGACAGTGTCATCCGCCTCCACCAAGGGCCC |
| | | ATCGGTCTTCCCCCTGGCGCCCTGCTCCAGGAGCACCTCCGAGA |
| | | GCACAGCCGCCCTGGGCTGCCTGGTCAAGGACTACTTCCCCGA |
| | | ACCGGTGACGGTGTCGTGGAACTCAGGCGCCCTGACCAGCGGC |
| | | GTGCACACCTTCCCGGCTGTCCTACAGTCCTCAGGACTCTACTC |
| | | CCTCAGCAGCGTGGTGACCGTGCCCTCCAGCAGCTTGGGCACG |
| | | AAGACCTACACCTGCAACGTAGATCACAAGCCCAGCAACACCA |
| | | AGGTGGACAAGAGAGTTGAGTCCAAATATGGTCCCCCATGCCC |
| | | ACCGTGCCCAGCACCACCTGTGGCAGGACCATCAGTCTTCCTGT |
| | | TCCCCCCAAAACCCAAGGACACTCTCATGATCTCCCGGACCCCT |
| | | GAGGTCACGTGCGTGGTGGTGGACGTGAGCCAGGAAGACCCCG |
| | | AGGTCCAGTTCAACTGGTACGTGGATGGCGTGGAGGTGCATAA |
| | | TGCCAAGACAAAGCCGCGGGAGGAGCAGTTCAACAGCACGTA |
| | | CCGTGTGGTCAGCGTCCTCACCGTCCTGCACCAGGACTGGCTG |
| | | AACGGCAAGGAGTACAAGTGCAAGGTCTCCAACAAAGGCCTCC |
| | | CGTCCTCCATCGAGAAAACCATCTCCAAAGCCAAAGGGCAGCC |
| | | CCGAGAGCCACAGGTGTACACCCTGCCCCCATCCCAGGAGGAG |
| | | ATGACCAAGAACCAGGTCAGCCTGACCTGCCTGGTCAAAGGCT |
| | | TCTACCCCAGCGACATCGCCGTGGAGTGGGAGAGCAATGGGCA |
| | | GCCGGAGAACAACTACAAGACCACGCCTCCCGTGCTGGACTCC |
| | | GACGGCTCCTTCTTCCTCTACAGCAGGCTCACCGTGGACAAGA |
| | | GCAGGTGGCAGGAGGGGAATGTCTTCTCATGCTCCGTGATGCA |
| | | TGAGGCTCTGCACAACCACTACACACAGAAGTCCCTCTCCCTGT |
| | | CTCTGGGTAAATGA |
| 4 | Anti-CD28 | MHRPRRRGTRPPPLALLAALLLAARGADAQVQLKQSGAELVKPG |
| | IgG4 Moiety | ASVKISCKTSGYTFTDGYMNWVEQKPGQGLEWIGRIDPDSGNTRY |
| | Used in the | NQKFQGKATLTRDKSSSTVYMDLRSLTSEDSAVYYCARDGTFYG |
| | Multispecific | TYGYWYFDFWGQGTQVTVSSASTKGPSVFPLAPCSRSTSESTAAL |
| | Molecule | GCLVKDYFPEPVTVSWNSGALTSGVHTFPAVLQSSGLYSLSSVVT |
| | Amino Acid | VPSSSLGTKTYTCNVDHKPSNTKVDKRVESKYGPPCPPCPAPPVA |
| | Sequence | GPSVFLFPPKPKDTLMISRTPEVTCVVVDVSQEDPEVQFNWYVDG |
| | (underline | VEVHNAKTKPREEQFNSTYRVVSVLTVLHQDWLNGKEYKCKVS |
| | portion is | NKGLPSSIEKTISKAKGQPREPQVYTLPPSQEEMTKNQVSLTCLVK |
| | mROR signal | GFYPSDIAVEWESNGQPENNYKTTPPVLDSDGSFFLYSRLTVDKSR |
| | sequence) | WQEGNVFSCSVMHEALHNHYTQKSLSLSLGK* |
| 5 | PiG GP33 | AAAGCTGTGTACAATTTCGCCACCTGT |
| | Peptide | |
| | Nucleic Acid | |
| | Sequence | |
| 6 | PiG GP33 | KAVYNFATM |
| | Peptide | |
| | Amino Acid | |
| | Sequence | |
| 7 | Beta-2 | ATCCAGAAAACCCCTCAAATTCAAGTATACTCACGCCACCCAC |
| | Microglobulin | CGGAGAATGGGAAGCCGAACATACTGAACTGCTACGTAACACA |
| | Nucleic Acid | GTTCCACCCGCCTCACATTGAAATCCAAATGCTGAAGAACGGG |
| | Sequence | AAAAAAATTCCTAAAGTAGAGATGTCAGATATGTCCTTCAGCA |
| | | AGGACTGGTCTTTCTATATCCTGGCTCACACTGAGTTCACCCCC |
| | | ACTGAGACTGATACATACGCCTGCAGAGTTAAGCATGCCAGTA |
| | | TGGCCGAGCCCAAGACCGTCTACTGGGATCGAGACATG |
| 8 | Beta-2 | IQKTPQIQVYSRHPPENGKPNILNCYVTQFHPPHIEIQMLKNGKKIP |
| | Microglobulin | KVEMSDMSFSKDWSFYILAHTEFTPTETDTYACRVKHASMAEPKT |
| | Amino Acid | VYWDRDM |
| | Sequence | |
| 9 | H2-Db MHC | GGCCCACACTCGATGCGGTATTTCGAGACCGCCGTGTCCCGGC |
| | Nucleic Acid | CCGGCCTCGAGGAGCCCCGGTACATCTCTGTCGGCTATGTGGA |
| | Sequence | CAACAAGGAGTTCGTGCGCTTCGACAGCGACGCGGAGAATCCG |
| | | AGATATGAGCCGCGGGCGCCGTGGATGGAGCAGGAGGGGCCG |
| | | GAGTATTGGGAGCGGGAAACACAGAAAGCCAAGGGCCAAGAG |
| | | CAGTGGTTCCGAGTGAGCCTGAGGAACCTGCTCGGCTGCTACA |
| | | ACCAGAGCGCGGGCGGCTCTCACACACTCCAGCAGATGTCTGG |
| | | CTGTGACTTGGGGTCGGACTGGCGCCTCCTCCGCGGGTACCTGC |
| | | AGTTCGCCTATGAAGGCCGCGATTACATCGCCCTGAACGAAGA |
| | | CCTGAAAACGTGGACGGCGGCGGACATGGCGGCGCAGATCAC |
| | | CCGACGCAAGTGGGAGCAGAGTGGTGCTGCAGAGCATTACAA |
| | | GGCCTACCTGGAGGGCGAGTGCGTGGAGTGGCTCCACAGATAC |
| | | CTGAAGAACGGGAACGCGACGCTGCTGCGCACAGATTCCCCAA |
| | | AGGCACATGTGACCCATCACCCCAGATCTAAAGGTGAAGTCAC |
| | | CCTGAGGTGCTGGGCCCTGGGCTTCTACCCTGCTGACATCACCC |
| | | TGACCTGGCAGTTGAATGGGGAGGAGCTGACCCAGGACATGGA |
| | | GCTTGTGGAGACCAGGCCTGCAGGGGATGGAACCTTCCAGAAG |
| | | TGGGCATCTGTGGTGGTGCCTCTTGGGAAGGAGCAGAATTACA |
| | | CATGCCGTGTGTACCATGAGGGGCTGCCTGAGCCCCTCACCCT |
| | | GAGATGGGAGCCTCCTCCGTCCACTGACTCTTACATGGTG |
| 10 | H2-Db MHC | GPHSMRYFETAVSRPGLEEPRYISVGYVDNKEFVRFDSDAENPRY |
| | Amino Acid | EPRAPWMEQEGPEYWERETQKAKGQEQWFRVSLRNLLGCYNQS |
| | Sequence | AGGSHTLQQMSGCDLGSDWRLLRGYLQFAYEGRDYIALNEDLKT |
| | | WTAADMAAQITRRKWEQSGAAEHYKAYLEGECVEWLHRYLKN |
| | | GNATLLRTDSPKAHVTHHPRSKGEVTLRCWALGFYPADITLTWQ |
| | | LNGEELTQDMELVETRPAGDGTFQKWASVVVPLGKEQNYTCRV |
| | | YHEGLPEPLTLRWEPPPSTDSYMV |
| 11 | GP33 PiG- | ATGCACAGACCAAGAAGAAGAGGAACTAGACCACCACCACTT |
| | IgG4 Fc | GCACTCCTTGCCGCCCTTTTGCTTGCCGCCCGTGGTGCTGATGC |
| | Fusion | AAAAGCTGTGTACAATTTCGCCACCTGTGGATGCGGAGGTAGT |
| | Protein | GGGGGAGGTGGCTCCGGGGGAGGGGGATCCATCCAGAAAACC |
| | Nucleic Acid | CCTCAAATTCAAGTATACTCACGCCACCCACCGGAGAATGGGA |
| | Sequence | AGCCGAACATACTGAACTGCTACGTAACACAGTTCCACCCGCC |
| | (underline | TCACATTGAAATCCAAATGCTGAAGAACGGGAAAAAAATTCCT |
| | portion is | AAAGTAGAGATGTCAGATATGTCCTTCAGCAAGGACTGGTCTT |
| | mROR signal | TCTATATCCTGGCTCACACTGAGTTCACCCCCACTGAGACTGAT |
| | sequence) | ACATACGCCTGCAGAGTTAAGCATGCCAGTATGGCCGAGCCCA |
| | | AGACCGTCTACTGGGATCGAGACATGGGTGGCGGCGGTAGCGG |
| | | CGGGGGCGGAAGCGGCGGAGGAGGCTCCGGTGGGGGTGGTTC |
| | | AGGCCCACACTCGATGCGGTATTTCGAGACCGCCGTGTCCCGG |
| | | CCCGGCCTCGAGGAGCCCCGGTACATCTCTGTCGGCTATGTGG |
| | | ACAACAAGGAGTTCGTGCGCTTCGACAGCGACGCGGAGAATCC |
| | | GAGATATGAGCCGCGGGCGCCGTGGATGGAGCAGGAGGGGCC |
| | | GGAGTATTGGGAGCGGGAAACACAGAAAGCCAAGGGCCAAGA |
| | | GCAGTGGTTCCGAGTGAGCCTGAGGAACCTGCTCGGCTGCTAC |
| | | AACCAGAGCGCGGGCGGCTCTCACACACTCCAGCAGATGTCTG |
| | | GCTGTGACTTGGGGTCGGACTGGCGCCTCCTCCGCGGGTACCT |
| | | GCAGTTCGCCTATGAAGGCCGCGATTACATCGCCCTGAACGAA |
| | | GACCTGAAAACGTGGACGGCGGCGGACATGGCGGCGCAGATC |
| | | ACCCGACGCAAGTGGGAGCAGAGTGGTGCTGCAGAGCATTACA |
| | | AGGCCTACCTGGAGGGCGAGTGCGTGGAGTGGCTCCACAGATA |
| | | CCTGAAGAACGGGAACGCGACGCTGCTGCGCACAGATTCCCCA |
| | | AAGGCACATGTGACCCATCACCCCAGATCTAAAGGTGAAGTCA |
| | | CCCTGAGGTGCTGGGCCCTGGGCTTCTACCCTGCTGACATCACC |
| | | CTGACCTGGCAGTTGAATGGGGAGGAGCTGACCCAGGACATGG |
| | | AGCTTGTGGAGACCAGGCCTGCAGGGGATGGAACCTTCCAGAA |
| | | GTGGGCATCTGTGGTGGTGCCTCTTGGGAAGGAGCAGAATTAC |
| | | ACATGCCGTGTGTACCATGAGGGGCTGCCTGAGCCCCTCACCC |
| | | TGAGATGGGAGCCTCCTCCGTCCACTGACTCTTACATGGTGGGT |
| | | GGTGGCGGATCTGGCGGCGGCGGAAGCGGAGGGGGAGGCTCC |
| | | GGAGGAGGCGGCTCCGAATCCAAATACGGTCCCCCCTGCCCCC |
| | | CATGTCCCGCTCCACCTGTGGCTGGTCCCTCTGTTTTCCTTTTTC |
| | | CCCCTAAACCCAAAGATACCCTCATGATTTCCAGAACCCCCGA |
| | | GGTCACCTGCGTCGTCGTTGATGTAAGCCAAGAAGATCCCGAA |
| | | GTCCAGTTCAATTGGTATGTAGACGGTGTTGAAGTCCATAATGC |
| | | AAAAACAAAACCCAGAGAGGAACAGTTTAATTCAACCTATCGT |
| | | GTCGTTAGCGTACTCACCGTTCTTCATCAAGACTGGCTCAATGG |
| | | AAAAGAATATAAATGTAAAGTTAGCAACAAAGGTCTGCCCAGT |
| | | TCAATCGAAAAAACAATTAGCAAAGCCAAAGGCCAACCTCGCG |
| | | AACCCCAAGTCTATACCTTGCCCCCTTCTCAGGAAGAAATGAC |
| | | CAAAAACCAAGTTTCACTCACATGCCTCGTAAAAGGATTCTAT |
| | | CCATCAGACATTGCAGTAGAATGGGAATCTAACGGCCAACCTG |
| | | AAAATAATTACAAAACCACTCCTCCTGTCCTCGATTCTGACGGC |
| | | TCTTTTTTCCTTTACTCCAGATTGACTGTTGATAAATCCCGCTGG |
| | | CAGGAAGGTAACGTTTTTTCTTGTTCTGTGATGCACGAAGCCCT |
| | | CCATAACAGATTCACTCAAAAATCTCTTTCTCTCTCCCCTGGCA |
| | | AATAA |
| 12 | GP33 PiG- | MHRPRRRGTRPPPLALLAALLLAARGADAKAVYNFATCGCGGSG |
| | IgG4 Fc | GGGSGGGGSIQKTPQIQVYSRHPPENGKPNILNCYVTQFHPPHIEIQ |
| | Fusion | MLKNGKKIPKVEMSDMSFSKDWSFYILAHTEFTPTETDTYACRVK |
| | Protein | HASMAEPKTVYWDRDMGGGGSGGGGSGGGGSGGGGSGPHSMR |
| | Amino Acid | YFETAVSRPGLEEPRYISVGYVDNKEFVRFDSDAENPRYEPRAPW |
| | Sequence | MEQEGPEYWERETQKAKGQEQWFRVSLRNLLGCYNQSAGGSHT |
| | (underline | LQQMSGCDLGSDWRLLRGYLQFAYEGRDYIALNEDLKTWTAAD |
| | portion is | MAAQITRRKWEQSGAAEHYKAYLEGECVEWLHRYLKNGNATLL |
| | mROR signal | RTDSPKAHVTHHPRSKGEVTLRCWALGFYPADITLTWQLNGEELT |
| | sequence) | QDMELVETRPAGDGTFQKWASVVVPLGKEQNYTCRVYHEGLPEP |
| | | LTLRWEPPPSTDSYMVGGGGSGGGGSGGGGSGGGGSESKYGPPC |
| | | PPCPAPPVAGPSVFLFPPKPKDTLMISRTPEVTCVVVDVSQEDPEV |
| | | QFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQ |
| | | VSLTCLVKGFYPSDIAVEWESNGQPENNYKTTPPVLDSDGSFFLYS |
| | | RLTVDKSRWQEGNVFSCSVMHEALHNRFTQKSLSLSPGK* |
| 13 | Anti-CD28 | ATGCACAGACCTAGACGTCGTGGAACTCGTCCACCTCCACTGG |
| | IgG1 Moiety | CACTGCTCGCTGCTCTCCTCCTGGCTGCACGTGGTGCTGATGCA |
| | Used in the | CAGGTACAATTGAAACAAAGTGGCGCCGAACTGGTAAAACCTG |
| | Multispecific | GAGCAAGTGTTAAGATCAGCTGTAAAACATCAGGATATACATT |
| | Molecule | TACCGACGGATATATGAATTGGGTGGAACAAAAACCTGGACAG |
| | Nucleic Acid | GGGCTCGAATGGATTGGAAGGATTGACCCAGATAGCGGTAACA |
| | Sequence | CAAGATACAACCAGAAATTTCAAGGCAAAGCAACTCTTACACG |
| | | CGATAAATCATCATCAACTGTCTACATGGACCTGCGTTCCCTTA |
| | | CTTCCGAGGATTCAGCTGTTTATTATTGCGCACGGGACGGAAC |
| | | ATTTTATGGAACATACGGTTATTGGTATTTCGACTTCTGGGGCC |
| | | AAGGAACTCAAGTGACAGTGTCATCCGCCTCCACCAAGGGCCC |
| | | ATCGGTCTTCCCCCTGGCACCCTCCTCCAAGAGCACCTCTGGGG |
| | | GCACAGCGGCCCTGGGCTGCCTGGTCAAGGACTACTTCCCCGA |
| | | ACCGGTGACGGTGTCGTGGAACTCAGGCGCCCTGACCAGCGGC |
| | | GTGCACACCTTCCCGGCTGTCCTACAGTCCTCAGGACTCTACTC |
| | | CCTCAGCAGCGTGGTGACCGTGCCCTCCAGCAGCTTGGGCACC |
| | | CAGACCTACATCTGCAACGTGAATCACAAGCCCAGCAACACCA |
| | | AGGTGGACAAGAAAGTTGAGCCCAAATCTTGTGACAAAACTCA |
| | | CACATGCCCACCGTGCCCAGCACCTGAACTCCTGGGGGGACCG |
| | | TCAGTCTTCCTCTTCCCCCCAAAACCCAAGGACACCCTCATGAT |
| | | CTCCCGGACCCCTGAGGTCACATGCGTGGTGGTGGACGTGAGC |
| | | CACGAAGACCCTGAGGTCAAGTTCAACTGGTACGTGGACGGCG |
| | | TGGAGGTGCATAATGCCAAGACAAAGCCGCGGGAGGAGCAGT |
| | | ACAACAGCACGTACCGTGTGGTCAGCGTCCTCACCGTCCTGCA |
| | | CCAGGACTGGCTGAATGGCAAGGAGTACAAGTGCAAGGTCTCC |
| | | AACAAAGCCCTCCCAGCCCCCATCGAGAAAACCATCTCCAAAG |
| | | CCAAAGGGCAGCCCCGAGAACCACAGGTGTACACCCTGCCCCC |
| | | ATCCCGGGATGAGCTGACCAAGAACCAGGTCAGCCTGACCTGC |
| | | CTGGTCAAAGGCTTCTATCCCAGCGACATCGCCGTGGAGTGGG |
| | | AGAGCAATGGGCAGCCGGAGAACAACTACAAGACCACGCCTC |
| | | CCGTGCTGGACTCCGACGGCTCCTTCTTCCTCTACAGCAAGCTC |
| | | ACCGTGGACAAGAGCAGGTGGCAGCAGGGGAACGTCTTCTCAT |
| | | GCTCCGTGATGCATGAGGCTCTGCACAACCACTACACGCAGAA |
| | | GTCCCTCTCCCTGTCTCCGGGTAAATGA |
| 14 | Anti-CD28 | MHRPRRRGTRPPPLALLAALLLAARGADAQVQLKQSGAELVKPG |
| | IgG1 Moiety | ASVKISCKTSGYTFTDGYMNWVEQKPGQGLEWIGRIDPDSGNTRY |
| | Used in the | NQKFQGKATLTRDKSSSTVYMDLRSLTSEDSAVYYCARDGTFYG |
| | Multispecific | TYGYWYFDFWGQGTQVTVSSASTKGPSVFPLAPSSKSTSGGTAAL |
| | Molecule | GCLVKDYFPEPVTVSWNSGALTSGVHTFPAVLQSSGLYSLSSVVT |
| | Amino Acid | VPSSSLGTQTYICNVNHKPSNTKVDKKVEPKSCDKTHTCPPCPAPE |
| | Sequence | LLGGPSVFLFPPKPKDTLMISRTPEVTCVVVDVSHEDPEVKFNWY |
| | (underline | VDGVEVHNAKTKPREEQYNSTYRVVSVLTVLHQDWLNGKEYKC |
| | portion is | KVSNKALPAPIEKTISKAKGQPREPQVYTLPPSRDELTKNQVSLTC |
| | mROR signal | LVKGFYPSDIAVEWESNGQPENNYKTTPPVLDSDGSFFLYSKLTV |
| | sequence) | DKSRWQQGNVFSCSVMHEALHNHYTQKSLSLSPGK* |
| 15 | GP33 PiG- | ATGCACAGACCTAGACGTCGTGGAACTCGTCCACCTCCACTGG |
| | IgG1 Fc | CACTGCTCGCTGCTCTCCTCCTGGCTGCACGTGGTGCTGATGCA |
| | Fusion | AAAGCTGTGTACAATTTCGCCACCTGTGGATGCGGAGGTAGTG |
| | Protein | GGGGAGGTGGCTCCGGGGGAGGGGGATCCATCCAGAAAACCC |
| | Nucleic Acid | CTCAAATTCAAGTATACTCACGCCACCCACCGGAGAATGGGAA |
| | Sequence | GCCGAACATACTGAACTGCTACGTAACACAGTTCCACCCGCCT |
| | (underline | CACATTGAAATCCAAATGCTGAAGAACGGGAAAAAAATTCCTA |
| | portion is | AAGTAGAGATGTCAGATATGTCCTTCAGCAAGGACTGGTCTTT |
| | mROR signal | CTATATCCTGGCTCACACTGAGTTCACCCCCACTGAGACTGATA |
| | sequence) | CATACGCCTGCAGAGTTAAGCATGCCAGTATGGCCGAGCCCAA |
| | | GACCGTCTACTGGGATCGAGACATGGGTGGCGGCGGTAGCGGC |
| | | GGGGGCGGAAGCGGCGGAGGAGGCTCCGGTGGGGGTGGTTCA |
| | | GGCCCACACTCGATGCGGTATTTCGAGACCGCCGTGTCCCGGC |
| | | CCGGCCTCGAGGAGCCCCGGTACATCTCTGTCGGCTATGTGGA |
| | | CAACAAGGAGTTCGTGCGCTTCGACAGCGACGCGGAGAATCCG |
| | | AGATATGAGCCGCGGGCGCCGTGGATGGAGCAGGAGGGGCCG |
| | | GAGTATTGGGAGCGGGAAACACAGAAAGCCAAGGGCCAAGAG |
| | | CAGTGGTTCCGAGTGAGCCTGAGGAACCTGCTCGGCTGCTACA |
| | | ACCAGAGCGCGGGCGGCTCTCACACACTCCAGCAGATGTCTGG |
| | | CTGTGACTTGGGGTCGGACTGGCGCCTCCTCCGCGGGTACCTGC |
| | | AGTTCGCCTATGAAGGCCGCGATTACATCGCCCTGAACGAAGA |
| | | CCTGAAAACGTGGACGGCGGCGGACATGGCGGCGCAGATCAC |
| | | CCGACGCAAGTGGGAGCAGAGTGGTGCTGCAGAGCATTACAA |
| | | GGCCTACCTGGAGGGCGAGTGCGTGGAGTGGCTCCACAGATAC |
| | | CTGAAGAACGGGAACGCGACGCTGCTGCGCACAGATTCCCCAA |
| | | AGGCACATGTGACCCATCACCCCAGATCTAAAGGTGAAGTCAC |
| | | CCTGAGGTGCTGGGCCCTGGGCTTCTACCCTGCTGACATCACCC |
| | | TGACCTGGCAGTTGAATGGGGAGGAGCTGACCCAGGACATGGA |
| | | GCTTGTGGAGACCAGGCCTGCAGGGGATGGAACCTTCCAGAAG |
| | | TGGGCATCTGTGGTGGTGCCTCTTGGGAAGGAGCAGAATTACA |
| | | CATGCCGTGTGTACCATGAGGGGCTGCCTGAGCCCCTCACCCT |
| | | GAGATGGGAGCCTCCTCCGTCCACTGACTCTTACATGGTGGGT |
| | | GGTGGCGGATCTGGCGGCGGCGGAAGCGGAGGGGGAGGCTCC |
| | | GGAGGAGGCGGCTCCGACAAAACTCACACATGCCCACCGTGCC |
| | | CAGCACCTGAACTCCTGGGGGGACCGTCAGTCTTCCTCTTCCCC |
| | | CCAAAACCCAAGGACACCCTCATGATCTCCCGGACCCCTGAGG |
| | | TCACATGCGTGGTGGTGGACGTGAGCCACGAAGACCCTGAGGT |
| | | CAAGTTCAACTGGTACGTGGACGGCGTGGAGGTGCATAATGCC |
| | | AAGACAAAGCCGCGGGAGGAGCAGTACAACAGCACGTACCGT |
| | | GTGGTCAGCGTCCTCACCGTCCTGCACCAGGACTGGCTGAATG |
| | | GCAAGGAGTACAAGTGCAAGGTCTCCAACAAAGCCCTCCCAGC |
| | | CCCCATCGAGAAAACCATCTCCAAAGCCAAAGGGCAGCCCCGA |
| | | GAACCACAGGTGTACACCCTGCCCCCATCCCGGGATGAGCTGA |
| | | CCAAGAACCAGGTCAGCCTGACCTGCCTGGTCAAAGGCTTCTA |
| | | TCCCAGCGACATCGCCGTGGAGTGGGAGAGCAATGGGCAGCCG |
| | | GAGAACAACTACAAGACCACGCCTCCCGTGCTGGACTCCGACG |
| | | GCTCCTTCTTCCTCTACAGCAAGCTCACCGTGGACAAGAGCAG |
| | | GTGGCAGCAGGGGAACGTCTTCTCATGCTCCGTGATGCATGAG |
| | | GCTCTGCACAACCGCTTCACGCAGAAGTCCCTCTCCCTGTCTCC |
| | | GGGTAAATAG |
| 16 | GP33 PiG- | MHRPRRRGTRPPPLALLAALLLAARGADAKAVYNFATCGCGGSG |
| | IgG1 Fc | GGGSGGGGSIQKTPQIQVYSRHPPENGKPNILNCYVTQFHPPHIEIQ |
| | Fusion | MLKNGKKIPKVEMSDMSFSKDWSFYILAHTEFTPTETDTYACRVK |
| | Protein | HASMAEPKTVYWDRDMGGGGSGGGGSGGGGSGGGGSGPHSMR |
| | Amino Acid | YFETAVSRPGLEEPRYISVGYVDNKEFVRFDSDAENPRYEPRAPW |
| | Sequence | MEQEGPEYWERETQKAKGQEQWFRVSLRNLLGCYNQSAGGSHT |
| | (underline | LQQMSGCDLGSDWRLLRGYLQFAYEGRDYIALNEDLKTWTAAD |
| | portion is | MAAQITRRKWEQSGAAEHYKAYLEGECVEWLHRYLKNGNATLL |
| | mROR signal | RTDSPKAHVTHHPRSKGEVTLRCWALGFYPADITLTWQLNGEELT |
| | sequence) | QDMELVETRPAGDGTFQKWASVVVPLGKEQNYTCRVYHEGLPEP |
| | | LTLRWEPPPSTDSYMVGGGGSGGGGSGGGGSGGGGSDKTHTCPP |
| | | CPAPELLGGPSVFLFPPKPKDTLMISRTPEVTCVVVDVSHEDPEVK |
| | | FNWYVDGVEVHNAKTKPREEQYNSTYRVVSVLTVLHQDWLNGK |
| | | EYKCKVSNKALPAPIEKTISKAKGQPREPQVYTLPPSRDELTKNQV |
| | | SLTCLVKGFYPSDIAVEWESNGQPENNYKTTPPVLDSDGSFFLYSK |
| | | LTVDKSRWQQGNVFSCSVMHEALHNRFTQKSLSLSPGK* |
| 17 | anti-CD20 | CAAGTTCAGTTGCAACAGCCAGGCGCCGAACTCGTTAGACCCG |
| | scFv Nucleic | GTACTTCAGTCAAACTTTCCTGCAAAGCTAGCGGATATACCTTC |
| | Acid | ACCTCTTATTGGATGCATTGGATCAAACAGCGCCCTGGACAAG |
| | Sequence | GCCTGGAATGGATTGGCGTGATCGACCCTTCAGACAACTATAC |
| | | TAAATACAACCAGAAATTTAAAGGCAAGGCTACACTCACAGTA |
| | | GACACTTCCTCAAGTACCGCATACATGCAGTTGAGCTCTCTTAC |
| | | CTCTGAAGACTCTGCAGTATATTTTTGCGCTAGGGAAGGATATT |
| | | ACGGATCATCTCCATGGTTTGCCTATTGGGGGCAGGGCACTCTC |
| | | GTAACCGTTTCCTCTGGAGGTGGTGGAAGTGGAGGTGGTGGAT |
| | | CCGGTGGAGGTGGATCACAGATCGTAATGAGTCAGTCTCCTGC |
| | | CATACTGTCAGCATCTCCCGGCGAAAAGGTCACCATGACATGC |
| | | AGAGCACGTTCATCAGTCAGTTACATACATTGGTATCAGCAAA |
| | | AACCCGGCTCATCCCCCAAACCATGGATCTACGCAACATCTAA |
| | | TCTCGCATCCGGCGTACCAGGCAGATTTTCTGGGAGCGGTTCA |
| | | GGCACCAGTTATTCCCTCACAATTACACGGGTTGAAGCAGAGG |
| | | ATGCAGCAACATATTATTGTCAGCAATGGTCTTCAAAACCCCC |
| | | AACTTTCGGCGGCGGTACAAAACTTGAAATATGA |
| 18 | anti-CD20 | QVQLQQPGAELVRPGTSVKLSCKASGYTFTSYWMHWIKQRPGQG |
| | scFv Amino | LEWIGVIDPSDNYTKYNQKFKGKATLTVDTSSSTAYMQLSSLTSE |
| | Acid | DSAVYFCAREGYYGSSPWFAYWGQGTLVTVSSQIVMSQSPAILSA |
| | Sequence | SPGEKVTMTCRARSSVSYIHWYQQKPGSSPKPWIYATSNLASGVP |
| | | GRFSGSGSGTSYSLTITRVEAEDAATYYCQQWSSKPPTFGGGTKLE |
| | | I* |
| 19 | GP33 PiG- | ATGCACAGACCAAGAAGAAGAGGAACTAGACCACCACCACTT |
| | IgG4/2- | GCACTCCTTGCCGCCCTTTTGCTTGCCGCCCGTGGTGCTGATGC |
| | stealth Fc- | AAAAGCTGTGTACAATTTCGCCACCTGTGGATGCGGAGGTAGT |
| | anti-CD20 | GGGGGAGGTGGCTCCGGGGGAGGGGGATCTATCCAGAAAACC |
| | Nucleic Acid | CCTCAAATTCAAGTATACTCACGCCACCCACCGGAGAATGGGA |
| | Sequence | AGCCGAACATACTGAACTGCTACGTAACACAGTTCCACCCGCC |
| | (underline | TCACATTGAAATCCAAATGCTGAAGAACGGGAAAAAAATTCCT |
| | portion is | AAAGTAGAGATGTCAGATATGTCCTTCAGCAAGGACTGGTCTT |
| | mROR signal | TCTATATCCTGGCTCACACTGAGTTCACCCCCACTGAGACTGAT |
| | sequence) | ACATACGCCTGCAGAGTTAAGCATGCCAGTATGGCCGAGCCCA |
| | | AGACCGTCTACTGGGATCGAGACATGGGTGGCGGCGGTAGCGG |
| | | CGGGGGCGGAAGCGGCGGAGGAGGCTCCGGTGGGGGTGGTTC |
| | | AGGCCCACACTCGATGCGGTATTTCGAGACCGCCGTGTCCCGG |
| | | CCCGGCCTCGAGGAGCCCCGGTACATCTCTGTCGGCTATGTGG |
| | | ACAACAAGGAGTTCGTGCGCTTCGACAGCGACGCGGAGAATCC |
| | | GAGATATGAGCCGCGGGCGCCGTGGATGGAGCAGGAGGGGCC |
| | | GGAGTATTGGGAGCGGGAAACACAGAAAGCCAAGGGCCAAGA |
| | | GCAGTGGTTCCGAGTGAGCCTGAGGAACCTGCTCGGCTGCTAC |
| | | AACCAGAGCGCGGGCGGCTCTCACACACTCCAGCAGATGTCTG |
| | | GCTGTGACTTGGGGTCGGACTGGCGCCTCCTCCGCGGGTACCT |
| | | GCAGTTCGCCTATGAAGGCCGCGATTACATCGCCCTGAACGAA |
| | | GACCTGAAAACGTGGACGGCGGCGGACATGGCGGCGCAGATC |
| | | ACCCGACGCAAGTGGGAGCAGAGTGGTGCTGCAGAGCATTACA |
| | | AGGCCTACCTGGAGGGCGAGTGCGTGGAGTGGCTCCACAGATA |
| | | CCTGAAGAACGGGAACGCGACGCTGCTGCGCACAGATTCCCCA |
| | | AAGGCACATGTGACCCATCACCCCAGATCTAAAGGTGAAGTCA |
| | | CCCTGAGGTGCTGGGCCCTGGGCTTCTACCCTGCTGACATCACC |
| | | CTGACCTGGCAGTTGAATGGGGAGGAGCTGACCCAGGACATGG |
| | | AGCTTGTGGAGACCAGGCCTGCAGGGGATGGAACCTTCCAGAA |
| | | GTGGGCATCTGTGGTGGTGCCTCTTGGGAAGGAGCAGAATTAC |
| | | ACATGCCGTGTGTACCATGAGGGGCTGCCTGAGCCCCTCACCC |
| | | TGAGATGGGAGCCTCCTCCGTCCACTGACTCTTACATGGTGGGT |
| | | GGTGGCGGATCTGGCGGCGGCGGAAGCGGAGGGGGAGGCTCC |
| | | GGAGGAGGCGGCTCCGAATCCAAATACGGTCCCCCCTGCCCCC |
| | | CATGTCCCGCTCCACCTGTGGCTGGACCATCAGTCTTCCTGTTC |
| | | CCCCCAAAACCCAAGGACACTCTCATGATCTCCCGGACCCCTG |
| | | AGGTCACGTGCGTGGTGGTGGACGTGAGCCAGGAAGACCCCGA |
| | | GGTCCAGTTCAACTGGTACGTGGATGGCGTGGAGGTGCATAAT |
| | | GCCAAGACAAAGCCGCGGGAGGAGCAGTTCAACAGCACGTAC |
| | | CGTGTGGTCAGCGTCCTCACCGTCCTGCACCAGGACTGGCTGA |
| | | ACGGCAAGGAGTACAAGTGCAAGGTCTCCAACAAAGGCCTCCC |
| | | GTCCTCCATCGAGAAAACCATCTCCAAAGCCAAAGGGCAGCCC |
| | | CGAGAGCCACAGGTGTACACCCTGCCCCCATCCCAGGAGGAGA |
| | | TGACCAAGAACCAGGTCAGCCTGTGGTGCCTGGTCAAAGGCTT |
| | | CTACCCCAGCGACATCGCCGTGGAGTGGGAGAGCAATGGGCAG |
| | | CCGGAGAACAACTACAAGACCACGCCTCCCGTGCTGGACTCCG |
| | | ACGGCTCCTTCTTCCTCTACAGCAGGCTCACCGTGGACAAGAG |
| | | CAGGTGGCAGGAGGGGAATGTCTTCTCATGCTCCGTGATGCAT |
| | | GAGGCTCTGCACAACCACTACACACAGAAGTCCCTCTCCCTGT |
| | | CTCTGGGTAAAGGCGGAGGTGGATCTGGAGGGGGCGGATCTCA |
| | | AGTTCAGTTGCAACAGCCAGGCGCCGAACTCGTTAGACCCGGT |
| | | ACTTCAGTCAAACTTTCCTGCAAAGCTAGCGGATATACCTTCAC |
| | | CTCTTATTGGATGCATTGGATCAAACAGCGCCCTGGACAAGGC |
| | | CTGGAATGGATTGGCGTGATCGACCCTTCAGACAACTATACTA |
| | | AATACAACCAGAAATTTAAAGGCAAGGCTACACTCACAGTAGA |
| | | CACTTCCTCAAGTACCGCATACATGCAGTTGAGCTCTCTTACCT |
| | | CTGAAGACTCTGCAGTATATTTTTGCGCTAGGGAAGGATATTAC |
| | | GGATCATCTCCATGGTTTGCCTATTGGGGGCAGGGCACTCTCGT |
| | | AACCGTTTCCTCTGGAGGTGGTGGAAGTGGAGGTGGTGGATCC |
| | | GGTGGAGGTGGATCACAGATCGTAATGAGTCAGTCTCCTGCCA |
| | | TACTGTCAGCATCTCCCGGCGAAAAGGTCACCATGACATGCAG |
| | | AGCACGTTCATCAGTCAGTTACATACATTGGTATCAGCAAAAA |
| | | CCCGGCTCATCCCCCAAACCATGGATCTACGCAACATCTAATCT |
| | | CGCATCCGGCGTACCAGGCAGATTTTCTGGGAGCGGTTCAGGC |
| | | ACCAGTTATTCCCTCACAATTACACGGGTTGAAGCAGAGGATG |
| | | CAGCAACATATTATTGTCAGCAATGGTCTTCAAAACCCCCAACT |
| | | TTCGGCGGCGGTACAAAACTTGAAATATGA |
| 20 | GP33 PiG- | MHRPRRRGTRPPPLALLAALLLAARGADAKAVYNFATCGCGGSG |
| | IgG4/2- | GGGSGGGGSIQKTPQIQVYSRHPPENGKPNILNCYVTQFHPPHIEIQ |
| | stealth Fc- | MLKNGKKIPKVEMSDMSFSKDWSFYILAHTEFTPTETDTYACRVK |
| | anti-CD20 | HASMAEPKTVYWDRDMGGGGSGGGGSGGGGSGGGGSGPHSMR |
| | Amino Acid | YFETAVSRPGLEEPRYISVGYVDNKEFVRFDSDAENPRYEPRAPW |
| | Sequence | MEQEGPEYWERETQKAKGQEQWFRVSLRNLLGCYNQSAGGSHT |
| | (underline | LQQMSGCDLGSDWRLLRGYLQFAYEGRDYIALNEDLKTWTAAD |
| | portion is | MAAQITRRKWEQSGAAEHYKAYLEGECVEWLHRYLKNGNATLL |
| | mROR signal | RTDSPKAHVTHHPRSKGEVTLRCWALGFYPADITLTWQLNGEELT |
| | sequence) | QDMELVETRPAGDGTFQKWASVVVPLGKEQNYTCRVYHEGLPEP |
| | | LTLRWEPPPSTDSYMVGGGGSGGGGSGGGGSGGGGSESKYGPPC |
| | | PPCPAPPVAGPSVFLFPPKPKDTLMISRTPEVTCVVVDVSQEDPEV |
| | | QFNWYVDGVEVHNAKTKPREEQFNSTYRVVSVLTVLHQDWLNG |
| | | KEYKCKVSNKGLPSSIEKTISKAKGQPREPQVYTLPPSQEEMTKNQ |
| | | VSLWCLVKGFYPSDIAVEWESNGQPENNYKTTPPVLDSDGSFFLY |
| | | SRLTVDKSRWQEGNVFSCSVMHEALHNHYTQKSLSLSLGKGGGG |
| | | SGGGGSQVQLQQPGAELVRPGTSVKLSCKASGYTFTSYWMHWIK |
| | | QRPGQGLEWIGVIDPSDNYTKYNQKFKGKATLTVDTSSSTAYMQ |
| | | LSSLTSEDSAVYFCAREGYYGSSPWFAYWGQGTLVTVSSGGGGS |
| | | GGGGSGGGGSQIVMSQSPAILSASPGEKVTMTCRARSSVSYIHWY |
| | | QQKPGSSPKPWIYATSNLASGVPGRFSGSGSGTSYSLTITRVEAED |
| | | AATYYCQQWSSKPPTFGGGTKLEI* |
| 21 | scMHCova | ATGCACAGACCTAGACGTCGTGGAACTCGTCCACCTCCACTGG |
| | Nucleic Acid | CACTGCTCGCTGCTCTCCTCCTGGCTGCACGTGGTGCTGATGCA |
| | Sequence | AGTATAATCAACTTTGAAAAACTGGGATGCGGAGGTAGTGGGG |
| | (underline | GAGGTGGCTCCGGGGGAGGGGGATCCATCCAGAAAACCCCTCA |
| | portion is | AATTCAAGTATACTCACGCCACCCACCGGAGAATGGGAAGCCG |
| | mROR signal | AACATACTGAACTGCTACGTAACACAGTTCCACCCGCCTCACA |
| | sequence) | TTGAAATCCAAATGCTGAAGAACGGGAAAAAAATTCCTAAAGT |
| | | AGAGATGTCAGATATGTCCTTCAGCAAGGACTGGTCTTTCTATA |
| | | TCCTGGCTCACACTGAGTTCACCCCCACTGAGACTGATACATAC |
| | | GCCTGCAGAGTTAAGCATGCCAGTATGGCCGAGCCCAAGACCG |
| | | TCTACTGGGATCGAGACATGGGTGGCGGCGGTAGCGGCGGGGG |
| | | CGGAAGCGGCGGAGGAGGCTCCGGTGGGGGTGGTTCAGGCCC |
| | | ACACTCGCTGAGGTATTTCGTCACCGCCGTGTCCCGGCCCGGCC |
| | | TCGGGGAGCCCCGGTACATGGAAGTCGGCTACGTGGACGACAC |
| | | GGAGTTCGTGCGCTTCGACAGCGACGCGGAGAATCCGAGATAT |
| | | GAGCCGCGGGCGCGGTGGATGGAGCAGGAGGGGCCCGAGTAT |
| | | TGGGAGCGGGAGACACAGAAAGCCAAGGGCAATGAGCAGAGT |
| | | TTCCGAGTGGACCTGAGGACCCTGCTCGGCTGCTACAACCAGA |
| | | GCAAGGGCGGCTCTCACACTATTCAGGTGATCTCTGGCTGTGA |
| | | AGTGGGGTCCGACGGGCGACTCCTCCGCGGGTACCAGCAGTAC |
| | | GCCTACGACGGCTGCGATTACATCGCCCTGAACGAAGACCTGA |
| | | AAACGTGGACGGCGGCGGACATGGCGGCGCTGATCACCAAAC |
| | | ACAAGTGGGAGCAGGCTGGTGAAGCAGAGAGACTCAGGGCCT |
| | | ACCTGGAGGGCACGTGCGTGGAGTGGCTCCGCAGATACCTGAA |
| | | GAACGGGAACGCGACGCTGCTGCGCACAGATTCCCCAAAGGCC |
| | | CATGTGACCCATCACAGCAGACCTGAAGATAAAGTCACCCTGA |
| | | GGTGCTGGGCCCTGGGCTTCTACCCTGCTGACATCACCCTGACC |
| | | TGGCAGTTGAATGGGGAGGAGCTGATCCAGGACATGGAGCTTG |
| | | TGGAGACCAGGCCTGCAGGGGATGGAACCTTCCAGAAGTGGGC |
| | | ATCTGTGGTGGTGCCTCTTGGGAAGGAGCAGTATTACACATGC |
| | | CATGTGTACCATCAGGGGCTGCCTGAGCCCCTCACCCTGAGAT |
| | | GGGAGCCTCCTCCATCCACTGTCTCCAACATGGCGACCGTTGCT |
| | | GTTCTGGTTGTCCTTGGAGCTGCAATAGTCACTGGAGCTGTGGT |
| | | GGCTTTTGTGATGAAGATGAGAAGGAGAAACACAGGTGGAAA |
| | | AGGAGGGGACTATGCTCTGGCTCCAGGCTCCCAGACCTCTGAT |
| | | CTGTCTCTCCCAGATTGTAAAGTGATGGTTCATGACCCTCATTC |
| | | TCTAGCGTGA |
| 22 | scMHCova | MHRPRRRGTRPPPLALLAALLLAARGADASIINFEKLGCGGSGGG |
| | Amino Acid | GSGGGGSIQKTPQIQVYSRHPPENGKPNILNCYVTQFHPPHIEIQML |
| | Sequence | KNGKKIPKVEMSDMSFSKDWSFYILAHTEFTPTETDTYACRVKHA |
| | (underline | SMAEPKTVYWDRDMGGGGSGGGGSGGGGSGGGGSGPHSLRYFV |
| | portion is | TAVSRPGLGEPRYMEVGYVDDTEFVRFDSDAENPRYEPRARWME |
| | mROR signal | QEGPEYWERETQKAKGNEQSFRVDLRTLLGCYNQSKGGSHTIQVI |
| | sequence) | SGCEVGSDGRLLRGYQQYAYDGCDYIALNEDLKTWTAADMAALI |
| | | TKHKWEQAGEAERLRAYLEGTCVEWLRRYLKNGNATLLRTDSP |
| | | KAHVTHHSRPEDKVTLRCWALGFYPADITLTWQLNGEELIQDME |
| | | LVETRPAGDGTFQKWASVVVPLGKEQYYTCHVYHQGLPEPLTLR |
| | | WEPPPSTVSNMATVAVLVVLGAAIVTGAVVAFVMKMRRRNTGG |
| | | KGGDYALAPGSQTSDLSLPDCKVMVHDPHSLA* |
| 23 | OVA PiG-Fc- | ATGCACAGACCTAGACGTCGTGGAACTCGTCCACCTCCACTGG |
| | anti-CD20 | CACTGCTCGCTGCTCTCCTCCTGGCTGCACGTGGTGCTGATGCA |
| | Fusion | AGTATAATCAACTTTGAAAAACTGGGATGCGGAGGTAGTGGGG |
| | Protein | GAGGTGGCTCCGGGGGAGGGGGATCCATCCAGAAAACCCCTCA |
| | Nucleic Acid | AATTCAAGTATACTCACGCCACCCACCGGAGAATGGGAAGCCG |
| | Sequence | AACATACTGAACTGCTACGTAACACAGTTCCACCCGCCTCACA |
| | (underline | TTGAAATCCAAATGCTGAAGAACGGGAAAAAAATTCCTAAAGT |
| | portion is | AGAGATGTCAGATATGTCCTTCAGCAAGGACTGGTCTTTCTATA |
| | mROR signal | TCCTGGCTCACACTGAGTTCACCCCCACTGAGACTGATACATAC |
| | sequence) | GCCTGCAGAGTTAAGCATGCCAGTATGGCCGAGCCCAAGACCG |
| | | TCTACTGGGATCGAGACATGGGTGGCGGCGGTAGCGGCGGGGG |
| | | CGGAAGCGGCGGAGGAGGCTCCGGTGGGGGTGGTTCAGGCCC |
| | | ACACTCGCTGAGGTATTTCGTCACCGCCGTGTCCCGGCCCGGCC |
| | | TCGGGGAGCCCCGGTACATGGAAGTCGGCTACGTGGACGACAC |
| | | GGAGTTCGTGCGCTTCGACAGCGACGCGGAGAATCCGAGATAT |
| | | GAGCCGCGGGCGCGGTGGATGGAGCAGGAGGGGCCCGAGTAT |
| | | TGGGAGCGGGAGACACAGAAAGCCAAGGGCAATGAGCAGAGT |
| | | TTCCGAGTGGACCTGAGGACCCTGCTCGGCTGCTACAACCAGA |
| | | GCAAGGGCGGCTCTCATACCATACAAGTCATTAGCGGATGTGA |
| | | AGTGGGGTCCGACGGGCGACTCCTCCGCGGGTACCAGCAGTAC |
| | | GCCTACGACGGCTGCGATTACATCGCCCTGAACGAAGACCTGA |
| | | AAACGTGGACGGCGGCGGACATGGCGGCGCTGATCACCAAAC |
| | | ACAAGTGGGAGCAGGCTGGTGAAGCAGAGAGACTCAGGGCCT |
| | | ACCTGGAGGGCACGTGCGTGGAGTGGCTCCGCAGATACCTGAA |
| | | GAACGGGAACGCGACGCTGCTGCGCACAGATTCCCCAAAGGCC |
| | | CATGTGACCCATCACAGCAGACCTGAAGATAAAGTCACCCTGA |
| | | GGTGCTGGGCCCTGGGCTTCTACCCTGCTGACATCACCCTGACC |
| | | TGGCAGTTGAATGGGGAGGAGCTGATCCAGGACATGGAGCTTG |
| | | TGGAGACCAGGCCTGCAGGGGATGGAACCTTCCAGAAGTGGGC |
| | | GTCTGTCGTGGTGCCGCTCGGTAAAGAGCAGTACTATACCTGC |
| | | CACGTATACCACCAGGGCTTGCCGGAACCATTGACATTGAGGT |
| | | GGGAACCTCCGCCCTCAACAGTCTCTAATATGGCTGGGGGGGG |
| | | AGGCAGTGGAGGCGGTGGTTCAGGTGGTGGTGGGTCCGGGGG |
| | | AGGAGGATCTGAATCTAAATATGGCCCCCCTTGTCCACCGTGC |
| | | CCTGCTCCACCTGTGGCTGGACCATCAGTCTTCCTGTTCCCCCC |
| | | AAAACCCAAGGACACTCTCATGATCTCCCGGACCCCTGAGGTC |
| | | ACGTGCGTGGTGGTGGACGTGAGCCAGGAAGACCCCGAGGTCC |
| | | AGTTCAACTGGTACGTGGATGGCGTGGAGGTGCATAATGCCAA |
| | | GACAAAGCCGCGGGAGGAGCAGTTCAACAGCACGTACCGTGT |
| | | GGTCAGCGTCCTCACCGTCCTGCACCAGGACTGGCTGAACGGC |
| | | AAGGAGTACAAGTGCAAGGTCTCCAACAAAGGCCTCCCGTCCT |
| | | CCATCGAGAAAACCATCTCCAAAGCCAAAGGGCAGCCCCGAG |
| | | AGCCACAGGTGTACACCCTGCCCCCATCCCAGGAGGAGATGAC |
| | | CAAGAACCAGGTCAGCCTGTGGTGCCTGGTCAAAGGCTTCTAC |
| | | CCCAGCGACATCGCCGTGGAGTGGGAGAGCAATGGGCAGCCG |
| | | GAGAACAACTACAAGACCACGCCTCCCGTGCTGGACTCCGACG |
| | | GCTCCTTCTTCCTCTACAGCAGGCTCACCGTGGACAAGAGCAG |
| | | GTGGCAGGAGGGGAATGTCTTCTCATGCTCCGTGATGCATGAG |
| | | GCTCTGCACAACCACTACACACAGAAGTCCCTCTCCCTGTCTCT |
| | | GGGTAAAGGCGGAGGTGGATCTGGAGGGGGCGGATCTCAAGT |
| | | TCAGTTGCAACAGCCAGGCGCCGAACTCGTTAGACCCGGTACT |
| | | TCAGTCAAACTTTCCTGCAAAGCTAGCGGATATACCTTCACCTC |
| | | TTATTGGATGCATTGGATCAAACAGCGCCCTGGACAAGGCCTG |
| | | GAATGGATTGGCGTGATCGACCCTTCAGACAACTATACTAAAT |
| | | ACAACCAGAAATTTAAAGGCAAGGCTACACTCACAGTAGACAC |
| | | TTCCTCAAGTACCGCATACATGCAGTTGAGCTCTCTTACCTCTG |
| | | AAGACTCTGCAGTATATTTTTGCGCTAGGGAAGGATATTACGG |
| | | ATCATCTCCATGGTTTGCCTATTGGGGGCAGGGCACTCTCGTAA |
| | | CCGTTTCCTCTGGAGGTGGTGGAAGTGGAGGTGGTGGTTCCGG |
| | | TGGAGGTGGATCACAGATCGTAATGAGTCAGTCTCCTGCCATA |
| | | CTGTCAGCATCTCCCGGCGAAAAGGTCACCATGACATGCAGAG |
| | | CACGTTCATCAGTCAGTTACATACATTGGTATCAGCAAAAACC |
| | | CGGCTCATCCCCCAAACCATGGATCTACGCAACATCTAATCTCG |
| | | CATCCGGCGTACCAGGCAGATTTTCTGGGAGCGGTTCAGGCAC |
| | | CAGTTATTCCCTCACAATTACACGGGTTGAAGCAGAGGATGCA |
| | | GCAACATATTATTGTCAGCAATGGTCTTCAAAACCCCCAACTTT |
| | | CGGCGGCGGTACAAAACTTGAAATATGA |
| 24 | OVA PiG-Fc- | MHRPRRRGTRPPPLALLAALLLAARGADASIINFEKLGCGGSGGG |
| | anti-CD20 | GSGGGGSIQKTPQIQVYSRHPPENGKPNILNCYVTQFHPPHIEIQML |
| | Fusion | KNGKKIPKVEMSDMSFSKDWSFYILAHTEFTPTETDTYACRVKHA |
| | Protein | SMAEPKTVYWDRDMGGGGSGGGGSGGGGSGGGGSGPHSLRYFV |
| | Amino Acid | TAVSRPGLGEPRYMEVGYVDDTEFVRFDSDAENPRYEPRARWME |
| | Sequence | QEGPEYWERETQKAKGNEQSFRVDLRTLLGCYNQSKGGSHTIQVI |
| | (underline | SGCEVGSDGRLLRGYQQYAYDGCDYIALNEDLKTWTAADMAALI |
| | portion is | TKHKWEQAGEAERLRAYLEGTCVEWLRRYLKNGNATLLRTDSP |
| | mROR signal | KAHVTHHSRPEDKVTLRCWALGFYPADITLTWQLNGEELIQDME |
| | sequence) | LVETRPAGDGTFQKWASVVVPLGKEQYYTCHVYHQGLPEPLTLR |
| | | WEPPPSTVSNMAGGGGSGGGGSGGGGSGGGGSESKYGPPCPPCP |
| | | APPVAGPSVFLFPPKPKDTLMISRTPEVTCVVVDVSQEDPEVQFN |
| | | WYVDGVEVHNAKTKPREEQFNSTYRVVSVLTVLHQDWLNGKEY |
| | | KCKVSNKGLPSSIEKTISKAKGQPREPQVYTLPPSQEEMTKNQVSL |
| | | WCLVKGFYPSDIAVEWESNGQPENNYKTTPPVLDSDGSFFLYSRL |
| | | TVDKSRWQEGNVFSCSVMHEALHNHYTQKSLSLSLGKGGGGSGG |
| | | GGSQVQLQQPGAELVRPGTSVKLSCKASGYTFTSYWMHWIKQRP |
| | | GQGLEWIGVIDPSDNYTKYNQKFKGKATLTVDTSSSTAYMQLSSL |
| | | TSEDSAVYFCAREGYYGSSPWFAYWGQGTLVTVSSGGGGSGGGG |
| | | SGGGGSQIVMSQSPAILSASPGEKVTMTCRARSSVSYIHWYQQKP |
| | | GSSPKPWIYATSNLASGVPGRFSGSGSGTSYSLTITRVEAEDAATY |
| | | YCQQWSSKPPTFGGGTKLEI* |
| 25 | scMHC- | ATGCACAGACCTAGACGTCGTGGAACTCGTCCACCTCCACTGG |
| | P15E | CACTGCTCGCTGCTCTCCTCCTGGCTGCACGTGGTGCTGATGCA |
| | Nucleic Acid | AAGAGCCCTTGGTTTACCACTTTGGGATGCGGAGGTAGTGGGG |
| | Sequence | GAGGTGGCTCCGGGGGAGGGGGATCCATCCAGAAAACCCCTCA |
| | (underline | AATTCAAGTATACTCACGCCACCCACCGGAGAATGGGAAGCCG |
| | portion is | AACATACTGAACTGCTACGTAACACAGTTCCACCCGCCTCACA |
| | mROR signal | TTGAAATCCAAATGCTGAAGAACGGGAAAAAAATTCCTAAAGT |
| | sequence) | AGAGATGTCAGATATGTCCTTCAGCAAGGACTGGTCTTTCTATA |
| | | TCCTGGCTCACACTGAGTTCACCCCCACTGAGACTGATACATAC |
| | | GCCTGCAGAGTTAAGCATGCCAGTATGGCCGAGCCCAAGACCG |
| | | TCTACTGGGATCGAGACATGGGTGGCGGCGGTAGCGGCGGGGG |
| | | CGGAAGCGGCGGAGGAGGCTCCGGTGGGGGTGGTTCAGGCCC |
| | | ACACTCGCTGAGGTATTTCGTCACCGCCGTGTCCCGGCCCGGCC |
| | | TCGGGGAGCCCCGGTACATGGAAGTCGGCTACGTGGACGACAC |
| | | GGAGTTCGTGCGCTTCGACAGCGACGCGGAGAATCCGAGATAT |
| | | GAGCCGCGGGCGCGGTGGATGGAGCAGGAGGGGCCCGAGTAT |
| | | TGGGAGCGGGAGACACAGAAAGCCAAGGGCAATGAGCAGAGT |
| | | TTCCGAGTGGACCTGAGGACCCTGCTCGGCTGCTACAACCAGA |
| | | GCAAGGGCGGCTCTCACACTATTCAGGTGATCTCTGGCTGTGA |
| | | AGTGGGGTCCGACGGGCGACTCCTCCGCGGGTACCAGCAGTAC |
| | | GCCTACGACGGCTGCGATTACATCGCCCTGAACGAAGACCTGA |
| | | AAACGTGGACGGCGGCGGACATGGCGGCGCTGATCACCAAAC |
| | | ACAAGTGGGAGCAGGCTGGTGAAGCAGAGAGACTCAGGGCCT |
| | | ACCTGGAGGGCACGTGCGTGGAGTGGCTCCGCAGATACCTGAA |
| | | GAACGGGAACGCGACGCTGCTGCGCACAGATTCCCCAAAGGCC |
| | | CATGTGACCCATCACAGCAGACCTGAAGATAAAGTCACCCTGA |
| | | GGTGCTGGGCCCTGGGCTTCTACCCTGCTGACATCACCCTGACC |
| | | TGGCAGTTGAATGGGGAGGAGCTGATCCAGGACATGGAGCTTG |
| | | TGGAGACCAGGCCTGCAGGGGATGGAACCTTCCAGAAGTGGGC |
| | | ATCTGTGGTGGTGCCTCTTGGGAAGGAGCAGTATTACACATGC |
| | | CATGTGTACCATCAGGGGCTGCCTGAGCCCCTCACCCTGAGAT |
| | | GGGAGCCTCCTCCATCCACTGTCTCCAACATGGCGACCGTTGCT |
| | | GTTCTGGTTGTCCTTGGAGCTGCAATAGTCACTGGAGCTGTGGT |
| | | GGCTTTTGTGATGAAGATGAGAAGGAGAAACACAGGTGGAAA |
| | | AGGAGGGGACTATGCTCTGGCTCCAGGCTCCCAGACCTCTGAT |
| | | CTGTCTCTCCCAGATTGTAAAGTGATGGTTCATGACCCTCATTC |
| | | TCTAGCGTGA |
| 26 | scMHC- | MHRPRRRGTRPPPLALLAALLLAARGADAKSPWFTTLGCGGSGG |
| | P15E Amino | GGSGGGGSIQKTPQIQVYSRHPPENGKPNILNCYVTQFHPPHIEIQM |
| | Acid | LKNGKKIPKVEMSDMSFSKDWSFYILAHTEFTPTETDTYACRVKH |
| | Sequence | ASMAEPKTVYWDRDMGGGGSGGGGSGGGGSGGGGSGPHSLRYF |
| | (underline | VTAVSRPGLGEPRYMEVGYVDDTEFVRFDSDAENPRYEPRARWM |
| | portion is | EQEGPEYWERETQKAKGNEQSFRVDLRTLLGCYNQSKGGSHTIQ |
| | mROR signal | VISGCEVGSDGRLLRGYQQYAYDGCDYIALNEDLKTWTAADMA |
| | sequence) | ALITKHKWEQAGEAERLRAYLEGTCVEWLRRYLKNGNATLLRTD |
| | | SPKAHVTHHSRPEDKVTLRCWALGFYPADITLTWQLNGEELIQDM |
| | | ELVETRPAGDGTFQKWASVVVPLGKEQYYTCHVYHQGLPEPLTL |
| | | RWEPPPSTVSNMATVAVLVVLGAAIVTGAVVAFVMKMRRRNTG |
| | | GKGGDYALAPGSQTSDLSLPDCKVMVHDPHSLA* |
| 27 | Anti-CD28 | ATGCACAGACCTAGACGTCGTGGAACTCGTCCACCTCCACTGG |
| | kappa LC | CACTGCTCGCTGCTCTCCTCCTGGCTGCACGTGGTGCTGATGCA |
| | Nucleic Acid | GATATCGTGATGACACAATCTCCCTATTCTCTGGCAGTATCCGC |
| | Sequence | AGGCGAGAAAGTCACCATGTCCTGTCGCTCCTCCCAGTCACTTT |
| | (underline | ATTATTCCGGAATTAAGAAAAATCTGCTCGCTTGGTACCAACA |
| | portion is | GAAACCTGGCCAATCACCTAAACTTCTTATCTACTTTACTTCTA |
| | mROR signal | CTCGACTTCCAGGAGTGCCTGACAGATTTACAGGGTCCGGTTCT |
| | sequence) | GGAACAGACTACACACTCACTATTACCTCCGTGCAGGCCGAAG |
| | | ACATGGGCCATTACTTCTGTCAGCAAGGCATTTCCACCCCTTTG |
| | | ACATTCGGAGATGGAACAAAGCTGGAAATTAAACGAACTGTGG |
| | | CTGCACCATCTGTCTTCATCTTCCCGCCATCTGATGAGCAGTTG |
| | | AAATCTGGAACTGCCTCTGTTGTGTGCCTGCTGAATAACTTCTA |
| | | TCCCAGAGAGGCCAAAGTACAGTGGAAGGTGGATAACGCCCTC |
| | | CAATCGGGTAACTCCCAGGAGAGTGTCACAGAGCAGGACAGC |
| | | AAGGACAGCACCTACAGCCTCAGCAGCACCCTGACGCTGAGCA |
| | | AAGCAGACTACGAGAAACACAAAGTCTACGCCTGCGAAGTCAC |
| | | CCATCAGGGCCTGAGCTCGCCCGTCACAAAGAGCTTCAACAGG |
| | | GGAGAGTGTTAG |
| 28 | Anti-CD28 | MHRPRRRGTRPPPLALLAALLLAARGADADIVMTQSPYSLAVSAG |
| | kappa LC | EKVTMSCRSSQSLYYSGIKKNLLAWYQQKPGQSPKLLIYFTSTRLP |
| | Amino Acid | GVPDRFTGSGSGTDYTLTITSVQAEDMGHYFCQQGISTPLTFGDGT |
| | Sequence | KLEIKRTVAAPSVFIFPPSDEQLKSGTASVVCLLNNFYPREAKVQW |
| | (underline | KVDNALQSGNSQESVTEQDSKDSTYSLSSTLTLSKADYEKHKVYA |
| | portion is | CEVTHQGLSSPVTKSFNRGEC* |
| | mROR signal | |
| | sequence) | |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

What is claimed is:

1. A multispecific molecule that is capable of binding to an antigen-specific T cell receptor (TCR) expressed on a surface of a cell, said multispecific molecule comprising a first molecule and a second molecule, wherein:
the first molecule is a polypeptide comprising (i) a peptide (p) presented in the context of a major histocompatibility complex (MHC) molecule (pMHC complex), and (ii) a first multimerization domain, and
the second molecule is a polypeptide comprising (i) a domain that specifically binds a molecule expressed on the surface of the cell expressing the TCR, and (ii) a second multimerization domain.

2. The multispecific molecule of claim 1, wherein the cell expressing the TCR is a lymphocyte.

3. The multispecific molecule of claim 2, wherein the lymphocyte is a T-cell.

4. The multispecific molecule of claim 3, wherein the T-cell is a CD4+ T-cell.

5. The multispecific molecule of claim 3, wherein the T-cell is a CD8+ T-cell.

6. The multispecific molecule of any one of claims 1-5, wherein the molecule expressed on the surface of the cell is not the TCR.

7. The multispecific molecule of claim 6, wherein the molecule expressed on the surface of the cell is a T cell immunomodulatory molecule.

8. The multispecific molecule of any one of claims 1-7, wherein the first and/or the second multimerization domain comprises an immunoglobulin Fc domain.

9. The multispecific molecule of claim 8, wherein the first and/or the second Fc domain is a human IgG Fc domain.

10. The multispecific molecule of claim 9, wherein the first and/or the second Fc domain is a human IgG1 Fc domain or a human IgG4 Fc domain.

11. The multispecific molecule of claim 8, wherein the first and/or the second Fc domain is a human IgM Fc domain.

12. The multispecific molecule of any one of claims 8-11, wherein the first and second Fc domains are the same.

13. The multispecific molecule of any one of claims 8-11, wherein the first Fc domain and/or the second Fc domain comprises an amino acid sequence which facilitates purification of the multispecific molecule.

14. The multispecific molecule of claim 13, wherein the amino acid sequence which facilitates purification of the multispecific molecule is a knob-into-hole mutation.

15. The multispecific molecule of claim 13, wherein the amino acid sequence which facilitates purification of the multispecific molecule is an amino acid substitution that leads to a weak or no detectable binding to an Fc-binding affinity matrix.

16. The multispecific molecule of any one of claims 8-10, wherein the first and/or the second Fc domain exhibits enhanced Fcγ-receptor binding activity relative to wild-type human IgG1.

17. The multispecific molecule of any one of claims 1-16, wherein the domain of the second binding molecule that specifically binds a molecule expressed on the surface of the cell comprises a Fab or single-chain Fv (scFv).

18. The multispecific molecule of any one of claims 1-17, wherein the first and/or the second multimerization domain comprises a second domain that specifically binds a cell-surface molecule.

19. The multispecific molecule of claim 18, wherein the second domain specifically binds a B-cell surface molecule or a tumor-associated antigen.

20. The multispecific molecule of claim 19, wherein the B-cell surface molecule is at least one of CD5, CD19, CD20, CD21, CD22, CD23, CD24, CD25, CD27, CD30, CD34, CD35, CD38, CD180, and CD40.

21. The multispecific molecule of claim 20, wherein the B-cell surface molecule is CD20 or CD180.

22. The multispecific molecule of any one of claims 18-21, wherein the second domain comprises a scFv.

23. The multispecific molecule of claim 22, wherein the scFv is attached to the C-terminus of the first or the second multimerization domain.

24. The multispecific molecule of any one of claims 1-23, wherein the pMHC complex comprises (i) a class I MHC polypeptide or a fragment, mutant or derivative thereof and (ii) a β2 microglobulin polypeptide or a fragment, mutant or derivative thereof.

25. The multispecific molecule of claim 24, wherein the pMHC complex comprises a class I MHC polypeptide linked to a β2 microglobulin polypeptide via a peptide linker.

26. The multispecific molecule of any one of claims 1-25, wherein the pMHC complex comprises a human class I MHC polypeptide is at least one of HLA-A, HLA-B, HLA-C, HLA-E, HLA-F, or HLA-G.

27. The multispecific molecule of any one of claims 1-25, wherein the pMHC complex comprises a murine class I MHC polypeptide is at least one of H-2K, H-2D, H-2L, H-2Q, H-2M, or H-2T.

28. The multispecific molecule of any one of claims 1-27, wherein the first molecule comprises (i) a peptide, (ii) a β₂-microglobulin polypeptide, (iii) a class I MHC alpha chain polypeptide or a fragment, mutant, or derivative thereof, and (iv) an immunoglobulin (Ig) Fc domain.

29. The multispecific molecule of claim 28, wherein the first molecule comprises, from the N-terminus to the C-terminus, (i) a peptide, (ii) a β2-microglobulin polypeptide, (iii) a class I MHC alpha chain or a fragment, mutant or derivative thereof, and (iv) an Ig Fc domain.

30. The multispecific molecule of claim 29, wherein the first molecule comprises, from N-terminus to C-terminus, (i) a peptide, (ii) an optional first linker, (iii) a β₂-microglobulin polypeptide or a fragment, mutant, or derivative thereof, (iv) an optional second linker, (v) class I MHC alpha chain domains 1, 2 and/or 3 or fragments, mutants, or derivatives thereof, (v) an optional third linker, and (vi) an Ig Fc domain.

31. The multispecific molecule of any one of claims 1-23, wherein the pMHC complex comprises a class II MHC polypeptide or a fragment, mutant or derivative thereof.

32. The multispecific molecule of claim 31, wherein the pMHC complex comprises class II MHC alpha and beta chain polypeptides or a fragment, mutant or derivative thereof.

33. The multispecific molecule of claim 32, wherein the alpha and beta chain polypeptides are linked by a peptide linker.

34. The multispecific molecule of claim 32 or claim 33, wherein the alpha and beta chain polypeptides are from a human class II MHC is at least one of HLA DP, HLA-DR, and HLA-DQ.

35. The multispecific molecule of claim 32 or claim 33, wherein the alpha and beta chain polypeptides are from a murine H-2A or H-2E class II MHC complex.

36. The multispecific molecule of any one of claims 1-23 and 31-35, wherein the first molecule comprises (i) a peptide, (ii) a class II MHC alpha chain polypeptide or a fragment, mutant or derivative thereof, (iii) a class II MHC beta chain polypeptide or a fragment, mutant or derivative thereof, and (iv) an Ig Fc domain.

37. The multispecific molecule of any one of claims 1-23, wherein the pMHC complex comprises MHC class II alpha chain extracellular domains.

38. The multispecific molecule of any one of claims 1-23, wherein the pMHC complex comprises class II beta chain extracellular domains.

39. The multispecific molecule of claim 37 or claim 38, wherein the first molecule comprises, from the N-terminus to the C-terminus, (i) a peptide, (ii) class II MHC alpha chain extracellular domains or fragments, mutants or derivatives thereof, (iii) class II MHC beta chain extracellular domains or fragments, mutants or derivatives thereof, and (iv) an Ig Fc domain.

40. The multispecific molecule of claim 37 or claim 38, wherein the first molecule comprises, from the N-terminus to the C-terminus, (i) a peptide, (ii) class II MHC beta chain extracellular domains or fragments, mutants or derivatives thereof, (iii) class II MHC alpha chain extracellular domains or fragments, mutants or derivatives thereof, and (iv) an Ig Fc domain.

41. The multispecific molecule of claim 39, wherein the first molecule comprises, from the N-terminus to the C-terminus, (i) a peptide, (ii) an optional first linker, (iii) class II MHC alpha chain domains 1 and 2 or fragments, mutants or derivatives thereof, (iv) an optional second linker, (v) class II MHC beta chain domains 1 and 2 or fragments, mutants or derivatives thereof, (vi) an optional third linker, and (vii) an Ig Fc domain.

42. The multispecific molecule of claim 40, wherein the first molecule comprises, from the N-terminus to the C-terminus, (i) a peptide, (ii) an optional first linker, (iii) class II MHC beta chain domains 1 and 2 or fragments, mutants or derivatives thereof, (iv) an optional second linker, (v) class II MHC alpha chain domains 1 and 2 or fragments, mutants or derivatives thereof, (vi) an optional third linker, and (vii) an Ig Fc domain.

43. The multispecific molecule of any one of claims 7-42, wherein the T-cell immunomodulatory molecule is a T-cell co-stimulatory molecule.

44. The multispecific molecule of claim 43, wherein the T-cell co-stimulatory molecule is at least one of CD2, CD27, CD28, CD30, CD40L, CD226, DR3, galectin 9, GITR, HVEM, ICOS, LFA1, OX40, SLAM, TIM1, TIM2, 2B4, or 4-1BB.

45. The multispecific molecule of any one of claims 7-42, wherein the T-cell immunomodulatory molecule is a T-cell inhibitory molecule.

46. The multispecific molecule of claim 45, wherein the T-cell inhibitory molecule is at least one of BTLA, B7-1, B7-H1, CD160, CTLA4, LAG3, LAIR1, MHC-I, PD1, TIGIT, or TIM3.

47. The multispecific molecule of any one of claims 1-46, wherein the peptide consists of from about 5 to about 40 amino acid residues.

48. The multispecific molecule of claim 47, wherein the peptide consists of from about 6 to about 30 amino acid residues.

49. The multispecific molecule of claim 48, wherein the peptide consists of from about 8 to about 20 amino acid residues.

50. The multispecific molecule of claim 49, wherein the peptide consists of from about 9 to about 11 amino acid residues.

51. The multispecific molecule of any one of claims 1-50, wherein the peptide is derived from a viral antigen or a bacterial antigen.

52. The multispecific molecule of claim 51, wherein he viral antigen is derived from a virus, wherein the virus is adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, or zika.

53. The multispecific molecule of claim 51, wherein the bacterial antigen is derived from a bacterium, where in the bacterium is methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, or Clindamycin-resistant group B Streptococcus.

54. The multispecific molecule of any one of claims 1-50, wherein the peptide is derived from a tumor-associated antigen.

55. The multispecific molecule of claim 54, wherein the tumor-associated antigen is at least one of adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML 1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1,-A2,-A3,-A4,-A6,-A9,-A10,-A12,-C1, and C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, or WT-1.

56. The multispecific molecule of any one of claims 1-50, wherein the peptide is derived from an antigen associated with an autoimmune disorder.

57. The multispecific molecule of claim 56, wherein the antigen associated with an autoimmune disorder is gliadin, GAD 65, IA-2, insulin β chain, glatiramer acetate (GA), acetylcholine receptor (AChR), p205, insulin, thyroid-stimulating hormone, tyrosinase, TRP1, or myelin antigens.

58. The multispecific molecule of claim 56, wherein the antigen associated with an autoimmune disorder is IL-4R, IL-6R, or DLL4.

59. The multispecific molecule of any one of claims 8-58, wherein the first, the second, or both the first and second immunoglobulin Fc domains is capable of binding to another Fc domain or an Fcγ-receptor expressed on a T-cell, and clustering four or more T-cells bound to the multispecific molecule.

60. The multispecific molecule of any one of claims 8-59, wherein the first, the second, or both the first and second immunoglobulin Fc domains is capable of binding to an Fcγ-receptor expressed on a cell, and clustering about 1200 or more peptide-specific T-cells.

61. The multispecific molecule of any one of claims 1-60 that is a bispecific molecule.

62. A multimeric molecule comprising two or more of the multispecific molecules of any one of claims 1-61.

63. A liposome comprising the multispecific molecule of any one of claims 1-61.

64. The liposome of claim 63, wherein the multispecific molecule is multimerized.

65. A pharmaceutical composition comprising the multispecific molecule of any one of claims 1-61 or the multimeric molecule of any one of claim 62, the liposome of claim 63 or claim 64, and a pharmaceutically acceptable carrier or diluent.

66. A multispecific carrier that is capable of binding to an antigen-specific T cell receptor (TCR) expressed on a surface of a cell, said multispecific carrier comprising a plurality of first molecules and a plurality of second molecules, wherein:
each first molecule a peptide (p) presented in the context of a major histocompatibility complex (MHC) molecule (pMHC complex), and
each second molecule is a polypeptide comprising a domain that specifically binds a molecule expressed on the surface of the cell expressing the TCR.

67. The multispecific carrier of claim 66, wherein the cell expressing the TCR is a lymphocyte.

68. The multispecific carrier of claim 67, wherein the lymphocyte is a T-cell.

69. The multispecific carrier of claim 68, wherein the T-cell is a CD4+ T-cell.

70. The multispecific carrier of claim 68, wherein the T-cell is a CD8+ T-cell.

71. The multispecific carrier of any one of claims 66-70, wherein the molecule expressed on the surface of the cell is not the TCR.

72. The multispecific carrier of claim 71, wherein the molecule expressed on the surface of the cell is a T cell immunomodulatory molecule.

73. The multispecific carrier of any one of claims 66-72, wherein the domain that specifically binds a molecule expressed on the surface of the cell expressing the TCR is an antigen-binding domain.

74. The multispecific carrier of claim 73, wherein the antigen-binding domain is a monovalent antigen-binding domain.

75. The multispecific carrier of claim 73, wherein the antigen-binding domain is a bispecific antigen-binding domain.

76. The multispecific carrier of claim 73, wherein the antigen-binding domain is a Fab or single-chain Fv (scFv).

77. The multispecific carrier of any one of claims 66-72, wherein the domain that specifically binds a molecule expressed on the surface of the cell expressing the TCR is a small molecule, protein, fusion protein, peptide, aptamer, avimer, or derivatives or fragments thereof.

78. The multispecific carrier of any one of claims 72-77, wherein the T-cell immunomodulatory molecule is a T-cell co-stimulatory molecule.

79. The multispecific carrier of claim 78, wherein the T-cell co-stimulatory molecule is at least one of CD2, CD27, CD28, CD30, CD40L, CD226, DR3, galectin 9, GITR, HVEM, ICOS, LFA1, OX40, SLAM, TIM1, TIM2, 2B4, or 4-1BB.

80. The multispecific carrier of any one of claims 72-79, wherein the T-cell immunomodulatory molecule is a T-cell inhibitory molecule.

81. The multispecific carrier of claim 80, wherein the T-cell inhibitory molecule is at least one of BTLA, B7-1 and B7-H1, CD160, CTLA4, LAG3, LAIR1, MHC-I, PD1, TIGIT, or TIM3.

82. The multispecific carrier of any one of claims 66-72, wherein the domain that specifically binds a molecule expressed on the surface of the cell expressing the TCR is a T-cell co-stimulatory molecule, or a fragment or derivative thereof.

83. The multispecific carrier of claim 82, wherein the T-cell co-stimulatory molecule is at least one of CD80, CD86, CD40, ICOSL, CD70, OX40L, 4-1BBL, GITRL, LIGHT, TIM3, TIM4, ICAM1, or LFA3.

84. The multispecific carrier of any one of claims 66-72, wherein the domain that specifically binds a molecule expressed on the surface of the cell expressing the TCR is a T-cell inhibitory molecule, or a fragment or derivative thereof.

85. The multispecific carrier of claim 84, wherein the T-cell inhibitory molecule is at least one of CD80, CD86, PDL-1, PD-L2, B7-H3, B7-H4, HVEM, ILT3, or ILT4.

86. The multispecific carrier of any one of claims 66-72, wherein the domain that specifically binds a molecule expressed on the surface of the cell expressing the TCR is a T-cell adhesion molecule, or a fragment or derivative thereof.

87. The multispecific carrier of claim 86, wherein the T-cell adhesion molecule is ICAM.

88. The multispecific carrier of any one of claims 66-87, wherein the pMHC complex comprises (i) a class I MHC polypeptide or a fragment, mutant or derivative thereof, and optionally, (ii) a β2 microglobulin polypeptide or a fragment, mutant or derivative thereof.

89. The multispecific carrier of claim 88, wherein the pMHC complex comprises a class I MHC polypeptide linked to a β2 microglobulin polypeptide via a peptide linker.

90. The multispecific carrier of any one of claims 66-89, wherein the pMHC complex comprises a human class I MHC polypeptide HLA-A, HLA-B, HLA-C, HLA-E, HLA-F, or HLA-G.

91. The multispecific carrier of any one of claims 66-89, wherein the pMHC complex comprises a murine class I MHC polypeptide H-2K, H-2D, H-2L, H-2Q, H-2M, or H-2T.

92. The multispecific carrier of any one of claims 66-91, wherein the first molecule comprises (i) a peptide, (ii) a β₂-microglobulin polypeptide or a fragment, mutant or derivative thereof, (iii) a class I MHC alpha chain polypeptide or a fragment, mutant, or derivative thereof, and (iv) a transmembrane domain.

93. The multispecific carrier of claim 92, wherein the first molecule comprises, from the N-terminus to the C-terminus, (i) a peptide, (ii) a β2-microglobulin polypeptide or a fragment, mutant or derivative thereof, (iii) a class I MHC alpha chain or a fragment, mutant or derivative thereof, and (iv) a transmembrane domain.

94. The multispecific carrier of claim 93, wherein the first molecule comprises, from N-terminus to C-terminus, (i) a peptide, (ii) an optional first linker, (iii) a β₂-microglobulin polypeptide or a fragment, mutant or derivative thereof, (iv) an optional second linker, (v) class I MHC alpha chain domains 1, 2 and/or 3 or fragments, mutants or derivatives thereof, (vi) an optional third linker, and (vii) a transmembrane domain.

95. The multispecific carrier of any one of claims 92-94, wherein the transmembrane domain is a transmembrane of a class I MHC alpha chain, T cell receptor alpha chain, T cell receptor beta chain, T cell receptor zeta chain, CD28, CD3 epsilon, CD45, CD4, CD5, CD8, CD8α, CD9, CD16, CD22, CD33, CD37, CD40, CD64, CD80, CD86, CD134, CD137, or CD154.

96. The multispecific carrier of any one of claims 66-87, wherein the pMHC complex comprises a class II MHC polypeptide or a fragment, mutant or derivative thereof.

97. The multispecific carrier of claim 96, wherein the pMHC complex comprises class II MHC alpha and beta chain polypeptides or a fragment, mutant or derivative thereof.

98. The multispecific carrier of claim 97, wherein the alpha and beta chain polypeptides are linked by a peptide linker.

99. The multispecific carrier of claim 97 or claim 98, wherein the alpha and beta chain polypeptides are from a human class II MHC HLA DP, HLA-DR, or HLA-DQ.

100. The multispecific carrier of claim 97 or claim 98, wherein the alpha and beta chain polypeptides are from a murine H-2A or H-2E class II MHC complex.

101. The multispecific carrier of any one of claims 66-87 and 96-100, wherein the first molecule comprises (i) a peptide, (ii) a class II MHC alpha chain polypeptide or a fragment, mutant or derivative thereof, (iii) a class II MHC beta chain polypeptide or a fragment, mutant or derivative thereof, and (iv) at least one transmembrane domain.

102. The multispecific carrier of claim 101, wherein the class II MHC alpha chain and class II MHC beta chain share one transmembrane domain.

103. The multispecific carrier of claim 101, wherein the class II MHC alpha chain and class II MHC beta chain each have a separate transmembrane domain.

104. The multispecific carrier of claim 103, wherein the separate transmembrane domains are the same.

105. The multispecific carrier of claim 103, wherein the separate transmembrane domains are different.

106. The multispecific carrier of any one of claims 66-87, wherein the pMHC complex comprises MHC class II alpha chain extracellular and a transmembrane domain.

107. The multispecific carrier of any one of claims 66-87, wherein the pMHC complex comprises class II beta chain extracellular and a transmembrane domain.

108. The multispecific carrier of claim 106 or claim 107, wherein the first molecule comprises, from the N-terminus to the C-terminus, (i) a peptide, (ii) class II MHC alpha chain extracellular domains or fragments, mutants or derivatives thereof, (iii) class II MHC beta chain extracellular domains or fragments, mutants or derivatives thereof, and (iv) a transmembrane domain.

109. The multispecific carrier of claim 106 or claim 107, wherein the first molecule comprises, from the N-terminus to the C-terminus, (i) a peptide, (ii) class II MHC beta chain extracellular domains or fragments, mutants or derivatives thereof, (iii) class II MHC alpha chain extracellular domains or fragments, mutants or derivatives thereof, and (iv) a transmembrane domain.

110. The multispecific carrier of claim 108, wherein the first molecule comprises, from the N-terminus to the C-terminus, (i) a peptide, (ii) an optional first linker, (iii) class II MHC alpha chain domains 1 and 2 or fragments, mutants or derivatives thereof, (iv) an optional second linker, (v) class II MHC beta chain domains 1 and 2 or fragments, mutants or derivatives thereof, (vi) an optional third linker, and (vii) a transmembrane domain.

111. The multispecific carrier of claim 109, wherein the first molecule comprises, from the N-terminus to the C-terminus, (i) a peptide, (ii) an optional first linker, (iii) class II MHC beta chain domains 1 and 2 or fragments, mutants or derivatives thereof, (iv) an optional second linker, (v) class II MHC alpha chain domains 1 and 2 or fragments, mutants or derivatives thereof, (vi) an optional third linker, and (vii) a transmembrane domain

112. The multispecific carrier of any one of claims 101-111, wherein the transmembrane is a class II MHC alpha chain transmembrane domain, the class II MHC beta chain transmembrane domain, T cell receptor alpha chain, T cell receptor beta chain, T cell receptor zeta chain, CD28, CD3 epsilon, CD45, CD4, CD5, CD8, CD8α, CD9, CD16, CD22, CD33, CD37, CD40, CD64, CD80, CD86, CD134, CD137, or CD154.

113. The multispecific carrier of any one of claims 66-112, wherein the peptide consists of from about 5 to about 40 amino acid residues.

114. The multispecific carrier of claim 113, wherein the peptide consists of from about 6 to about 30 amino acid residues.

115. The multispecific carrier of claim 114, wherein the peptide consists of from about 8 to about 20 amino acid residues.

116. The multispecific carrier of claim 115, wherein the peptide consists of from about 9 to about 11 amino acid residues.

117. The multispecific carrier of any one of claims 66-116, wherein the peptide is derived from a viral antigen or a bacterial antigen.

118. The multispecific carrier of claim 117, wherein he viral antigen is derived from a virus, wherein the virus is adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, or zika.

119. The multispecific carrier of claim 117, wherein the bacterial antigen is derived from a bacterium, wherein the bacterium is methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum B-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, or Clindamycin-resistant group B Streptococcus.

120. The multispecific carrier of any one of claims 66-116, wherein the peptide is derived from a tumor-associated antigen.

121. The multispecific carrier of claim 120, wherein the tumor-associated antigen is adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRvIII, ErbB2/Her2, ErbB3, ErbB4, ETV6-AML 1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-A1,-A2,-A3,-A4,-A6,-A9,-A10,-A12,-C1, and C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, or WT-1.

122. The multispecific carrier of any one of claims 66-116, wherein the peptide is derived from an antigen associated with an autoimmune disorder.

123. The multispecific carrier of claim 122, wherein the antigen associated with an autoimmune disorder is gliadin, GAD 65, IA-2, insulin β chain, glatiramer acetate (GA), acetylcholine receptor (AChR), p205, insulin, thyroid-stimulating hormone, tyrosinase, TRP1, or myelin antigens.

124. The multispecific carrier of claim 122, wherein the antigen associated with an autoimmune disorder is IL-4R, IL-6R, or DLL4.

125. The multispecific carrier of any one of claims 66-124, wherein each first molecule of the plurality of first molecules is the same.

126. The multispecific carrier of any one of claims 66-125, wherein each first molecule of the plurality of first molecules comprises at least two, three, four, five, or six different types of first molecules.

127. The multispecific carrier of any one of claims 66-125, wherein each second molecule of the plurality of second molecules is the same.

128. The multispecific carrier of any one of claims 66-125, wherein each second molecule of the plurality of second molecules comprises at least two, three, four, five, or six different types of first molecules.

129. The multispecific carrier of any one of claims 66-128, wherein the carrier is a cell.

130. The multispecific carrier of claim 129, wherein the cell is CHO, HEK 293, HMEC, HIVE-55, HIVS-125, autologous cells, allogenic cells, or tumor cells.

131. The multispecific carrier of claim 129, wherein the cell is an antigen presenting cell.

132. The multispecific carrier of claim 131, wherein the cell is a B-cell.

133. The multispecific carrier of any one of claims 66-128, wherein the carrier is a virus-like particle.

134. The multispecific carrier of any one of claims 66-128, wherein the carrier is a recombinant virus.

135. The multispecific carrier of claim 134, wherein the virus is derived from a retrovirus, an adenovirus, an adeno-associated virus, or a herpesvirus.

136. The multispecific carrier of claim 135, wherein the retrovirus is a lentivirus.

137. A pharmaceutical composition comprising the multispecific carrier of any one of claims 66-136, and a pharmaceutically acceptable carrier or diluent.

138. A virus-like particle (VLP) comprising the multispecific molecule of any one of claims 1-61.

139. The virus-like particle (VLP) of claim 138, wherein the multispecific molecule is multimerized.

140. A pharmaceutical composition comprising the VLP of claim 138 or claim 139, and a pharmaceutically acceptable carrier or diluent.

141. A recombinant virus comprising the multispecific molecule of any one of claims 1-61.

142. The recombinant virus of claim 141, wherein the multispecific molecule is multimerized.

143. The recombinant virus of claim 141 or claim 142, wherein the virus is derived from a retrovirus, an adenovirus, an adeno-associated virus, or a herpesvirus.

144. The recombinant virus of claim 143, wherein the retrovirus is a lentivirus.

145. A pharmaceutical composition comprising the recombinant virus of any one of claims 141-144, and a pharmaceutically acceptable carrier or diluent.

146. A solid support comprising the multispecific molecule of any one of claims 1-61.

147. The solid support of claim 146, wherein the multispecific molecule is multimerized.

148. The solid support of claim 146 or claim 147 which is a bead.

149. An engineered cell comprising the multispecific molecule of any one of claims 1-61.

150. The engineered cell of claim 149, wherein the multispecific molecule is multimerized.

151. The engineered cell or claim 149 or claim 150 which is a B-cell.

152. A pharmaceutical composition comprising an engineered cell according to any one of claims 149-151 and a pharmaceutically acceptable carrier or diluent.

153. A single chain peptide-major histocompatibility complex (pMHC) fusion molecule that is capable of binding to an antigen-specific T cell receptor (TCR) expressed on a surface of a cell comprising (i) a peptide (p) presented in the context of an MHC molecule (pMHC complex) and (ii) a domain that specifically binds a molecule expressed on the surface of a carrier.

154. The single chain peptide-major histocompatibility complex (pMHC) fusion molecule of claim 153, wherein the cell expressing the TCR is a lymphocyte.

155. The single chain peptide-major histocompatibility complex (pMHC) fusion molecule of claim 154, wherein the lymphocyte is a T-cell.

156. The single chain peptide-major histocompatibility complex (pMHC) fusion molecule of claim 155, wherein the T-cell is a CD4+ T-cell.

157. The single chain peptide-major histocompatibility complex (pMHC) fusion molecule of claim 155, wherein the T-cell is a CD8+ T-cell.

158. The single chain peptide-major histocompatibility complex (pMHC) fusion molecule of any one of claims 153-157, wherein the domain that specifically binds a molecule expressed on the surface of the carrier comprises a monovalent antigen-binding domain, bispecific antigen-binding domain, Fab, Fab′, F(ab′) 2, Fv, single-chain Fv (scFv), disulfide-stabilized Fv (dsFv), ds-scFv, Fd, linear antibody, minibody, diabody, monovalent antibody fragment, bispecific antibody fragment, bibody, tribody, sc-diabody, kappa (lambda) body, BiTE, DVD-Ig, SIP, SMIP, or DART.

159. The single chain peptide-major histocompatibility complex (pMHC) fusion molecule of claim 158, wherein the domain that specifically binds a molecule expressed on the surface of the carrier comprises a Fab or scFv.

160. The single chain peptide-major histocompatibility complex (pMHC) fusion molecule of any one of claims 153-159, wherein the carrier is a cell, virus, virus-like particle, or solid support.

161. The single chain peptide-major histocompatibility complex (pMHC) fusion molecule of any one of claims 153-160, wherein the carrier is a cell.

162. The single chain peptide-major histocompatibility complex (pMHC) fusion molecule of claim 161, wherein the cell is CHO, HEK 293, HMEC, HIVE-55, HIVS-125, autologous cells, allogenic cells, or tumor cells.

163. The single chain peptide-major histocompatibility complex (pMHC) fusion molecule of claim 161 or claim 162, wherein the cell is an antigen presenting cell.

164. The single chain peptide-major histocompatibility complex (pMHC) fusion molecule of claim 163, wherein the cell is a B-cell.

165. The single chain peptide-major histocompatibility complex (pMHC) fusion molecule of claim 164, wherein the B-cell surface molecule is at least one of CD5, CD19, CD20, CD21, CD22, CD23, CD24, CD25, CD27, CD30, CD34, CD35, CD38, CD180, and CD40.

166. The single chain peptide-major histocompatibility complex (pMHC) fusion molecule of claim 165, wherein the B-cell surface molecule is CD20 or CD180.

167. The single chain peptide-major histocompatibility complex (pMHC) fusion molecule of any one of claims 153-160, wherein the carrier is a virus-like particle.

168. The single chain peptide-major histocompatibility complex (pMHC) fusion molecule of any one of claims 153-160, wherein the carrier is a recombinant virus.

169. The single chain peptide-major histocompatibility complex (pMHC) fusion molecule of claim 168, wherein the virus is derived from a retrovirus, an adenovirus, an adeno-associated virus, or a herpesvirus.

170. The single chain peptide-major histocompatibility complex (pMHC) fusion molecule of claim 168, wherein the retrovirus is a lentivirus.

171. The single chain peptide-major histocompatibility complex (pMHC) fusion molecule of any one of claims 153-170, wherein the pMHC complex comprises (i) a class I MHC polypeptide or a fragment, mutant or derivative thereof and (ii) a β2 microglobulin polypeptide or a fragment, mutant or derivative thereof.

172. The single chain peptide-major histocompatibility complex (pMHC) fusion molecule of claim 171, wherein the pMHC complex comprises a class I MHC polypeptide linked to a β2 microglobulin polypeptide via a peptide linker.

173. The single chain peptide-major histocompatibility complex (pMHC) fusion molecule of any one of claims 153-172, wherein the pMHC complex comprises a human class I MHC polypeptide is at least one of HLA-A, HLA-B, HLA-C, HLA-E, HLA-F, or HLA-G.

174. The single chain peptide-major histocompatibility complex (pMHC) fusion molecule of any one of claims 153-172, wherein the pMHC complex comprises a murine class I MHC polypeptide is at least one of H-2K, H-2D, H-2L, H-2Q, H-2M, or H-2T.

175. The single chain peptide-major histocompatibility complex (pMHC) fusion molecule of any one of claims 153-174, wherein the first molecule comprises (i) a peptide, (ii) a β₂-microglobulin polypeptide, (iii) a class I MHC alpha chain polypeptide or a fragment, mutant, or derivative thereof, and (iv) an immunoglobulin (Ig) Fc domain.

176. The single chain peptide-major histocompatibility complex (pMHC) fusion molecule of claim 175, wherein the first molecule comprises, from the N-terminus to the C-terminus, (i) a peptide, (ii) a β2-microglobulin polypeptide, (iii) a class I MHC alpha chain or a fragment, mutant or derivative thereof, and (iv) an Ig Fc domain.

177. The single chain peptide-major histocompatibility complex (pMHC) fusion molecule of claim 176, wherein the first molecule comprises, from N-terminus to C-terminus, (i) a peptide, (ii) an optional first linker, (iii) a β₂-microglobulin polypeptide or a fragment, mutant, or derivative thereof, (iv) an optional second linker, (v) class I MHC alpha chain domains 1, 2 and/or 3 or fragments, mutants, or derivatives thereof, (v) an optional third linker, and (vi) an Ig Fc domain.

178. The single chain peptide-major histocompatibility complex (pMHC) fusion molecule of any one of claims 153-170, wherein the pMHC complex comprises a class II MHC polypeptide or a fragment, mutant or derivative thereof.

179. The single chain peptide-major histocompatibility complex (pMHC) fusion molecule of claim 178, wherein the pMHC complex comprises class II MHC alpha and beta chain polypeptides or a fragment, mutant or derivative thereof.

180. The single chain peptide-major histocompatibility complex (pMHC) fusion molecule of claim 179, wherein the alpha and beta chain polypeptides are linked by a peptide linker.

181. The single chain peptide-major histocompatibility complex (pMHC) fusion molecule of claim 179 or claim 180, wherein the alpha and beta chain polypeptides are from a human class II MHC is at least one of HLA DP, HLA-DR, and HLA-DQ.

182. The single chain peptide-major histocompatibility complex (pMHC) fusion molecule of claim 179 or claim 180, wherein the alpha and beta chain polypeptides are from a murine H-2A or H-2E class II MHC complex.

183. The single chain peptide-major histocompatibility complex (pMHC) fusion molecule of any one of claims 153-170 and 178-182, wherein the first molecule comprises (i) a peptide, (ii) a class II MHC alpha chain polypeptide or a fragment, mutant or derivative thereof, (iii) a class II MHC beta chain polypeptide or a fragment, mutant or derivative thereof, and (iv) an Ig Fc domain.

184. The single chain peptide-major histocompatibility complex (pMHC) fusion molecule of any one of claims 153-170, wherein the pMHC complex comprises MHC class II alpha chain extracellular domains.

185. The single chain peptide-major histocompatibility complex (pMHC) fusion molecule of any one of claims 153-170, wherein the pMHC complex comprises class II beta chain extracellular domains.

186. The single chain peptide-major histocompatibility complex (pMHC) fusion molecule of claim 184 or claim 185, wherein the first molecule comprises, from the N-terminus to the C-terminus, (i) a peptide, (ii) class II MHC alpha chain extracellular domains or fragments, mutants or derivatives thereof, (iii) class II MHC beta chain extracellular domains or fragments, mutants or derivatives thereof, and (iv) an Ig Fc domain.

187. The single chain peptide-major histocompatibility complex (pMHC) fusion molecule of claim 184 or claim 185, wherein the first molecule comprises, from the N-terminus to the C-terminus, (i) a peptide, (ii) class II MHC beta chain extracellular domains or fragments, mutants or derivatives thereof, (iii) class II MHC alpha chain extracellular domains or fragments, mutants or derivatives thereof, and (iv) an Ig Fc domain.

188. The single chain peptide-major histocompatibility complex (pMHC) fusion molecule of claim 186, wherein the first molecule comprises, from the N-terminus to the C-terminus, (i) a peptide, (ii) an optional first linker, (iii) class II MHC alpha chain domains 1 and 2 or fragments, mutants or derivatives thereof, (iv) an optional second linker, (v) class II MHC beta chain domains 1 and 2 or fragments, mutants or derivatives thereof, (vi) an optional third linker, and (vii) an Ig Fc domain.

189. The single chain peptide-major histocompatibility complex (pMHC) fusion molecule of claim 187, wherein the first molecule comprises, from the N-terminus to the C-terminus, (i) a peptide, (ii) an optional first linker, (iii) class II MHC beta chain domains 1 and 2 or fragments, mutants or derivatives thereof, (iv) an optional second linker, (v) class II MHC alpha chain domains 1 and 2 or fragments, mutants or derivatives thereof, (vi) an optional third linker, and (vii) an Ig Fc domain.

190. The single chain peptide-major histocompatibility complex (pMHC) fusion molecule of any one of claims 153-189, wherein the peptide consists of from about 5 to about 40 amino acid residues.

191. The single chain peptide-major histocompatibility complex (pMHC) fusion molecule of claim 190, wherein the peptide consists of from about 6 to about 30 amino acid residues.

192. The single chain peptide-major histocompatibility complex (pMHC) fusion molecule of claim 191, wherein the peptide consists of from about 8 to about 20 amino acid residues.

193. The single chain peptide-major histocompatibility complex (pMHC) fusion molecule of claim 192, wherein the peptide consists of from about 9 to about 11 amino acid residues.

194. The single chain peptide-major histocompatibility complex (pMHC) fusion molecule of any one of claims 153-193, wherein the peptide is derived from a viral antigen or a bacterial antigen.

195. The single chain peptide-major histocompatibility complex (pMHC) fusion molecule of claim 194, wherein he viral antigen is derived from a virus, wherein the virus is adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, or zika.

196. The single chain peptide-major histocompatibility complex (pMHC) fusion molecule of claim 194, wherein the bacterial antigen is derived from a bacterium, where in the bacterium is methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum β-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, B Erythomycin-resistant group A Streptococcus, or Clindamycin-resistant group Streptococcus.

197. The single chain peptide-major histocompatibility complex (pMHC) fusion molecule of any one of claims 153-193, wherein the peptide is derived from a tumor-associated antigen.

198. The single chain peptide-major histocompatibility complex (pMHC) fusion molecule of claim 197, wherein the tumor-associated antigen is at least one of adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3α2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, LMP2, M-CSF, MAGE proteins (e.g., MAGE-Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, or WT-1.

199. The single chain peptide-major histocompatibility complex (pMHC) fusion molecule of any one of claims 153-193, wherein the peptide is derived from an antigen associated with an autoimmune disorder.

200. The single chain peptide-major histocompatibility complex (pMHC) fusion molecule of claim 199, wherein the antigen associated with an autoimmune disorder is gliadin, GAD 65, IA-2, insulin β chain, glatiramer acetate (GA), acetylcholine receptor (AChR), p205, insulin, thyroid-stimulating hormone, tyrosinase, TRP1, or myelin antigens.

201. The single chain peptide-major histocompatibility complex (pMHC) fusion molecule of claim 199, wherein the antigen associated with an autoimmune disorder is IL-4R, IL-6R, or DLL4.

202. The single chain peptide-major histocompatibility complex (pMHC) fusion molecule of any one of claims 153-201, wherein the single chain peptide-major histocompatibility complex (pMHC) fusion molecule is able to cluster four or more T-cells bound to the single chain major histocompatibility complex (MHC)-peptide.

203. A pharmaceutical composition comprising the single chain peptide-major histocompatibility complex (pMHC) fusion molecule of any one of claims 153-202, and a pharmaceutically acceptable carrier or diluent.

204. A single chain peptide-major histocompatibility complex (pMHC) fusion molecule carrier that is capable of binding to an antigen-specific T cell receptor (TCR) expressed on a surface of a cell, said multispecific carrier comprising a plurality of single chain major histocompatibility complex (MHC)-peptides, wherein each single chain major histocompatibility complex (MHC)-peptide comprises a peptide (p) presented in the context of a major histocompatibility complex (MHC) molecule (pMHC complex), and a domain that specifically binds a molecule expressed on the surface of the carrier.

205. The single chain peptide-major histocompatibility complex (pMHC) fusion molecule carrier of claim 204, wherein the cell expressing the TCR is a lymphocyte.

206. The single chain peptide-major histocompatibility complex (pMHC) fusion molecule carrier of claim 205, wherein the lymphocyte is a T-cell.

207. The single chain peptide-major histocompatibility complex (pMHC) fusion molecule carrier of claim 206, wherein the T-cell is a CD4+ T-cell.

208. The single chain peptide-major histocompatibility complex (pMHC) fusion molecule carrier of claim 206, wherein the T-cell is a CD8+ T-cell.

209. The single chain peptide-major histocompatibility complex (pMHC) fusion molecule carrier of any one of claims 204-208, wherein the carrier further comprises a T-cell immunomodulatory molecule.

210. The single chain peptide-major histocompatibility complex (pMHC) fusion molecule carrier of claim 209, wherein the is a T-cell co-stimulatory molecule.

211. The single chain peptide-major histocompatibility complex (pMHC) fusion molecule carrier of claim 210, wherein the T-cell co-stimulatory molecule is at least one ligand of CD2, CD27, CD28, CD30, CD40L, CD226, DR3, galectin 9, GITR, HVEM, ICOS, LFA1, OX40, SLAM, TIM1, TIM2, 2B4, or 4-1BB.

212. The single chain peptide-major histocompatibility complex (pMHC) fusion molecule carrier of claim 209, wherein the T-cell immunomodulatory molecule is a T-cell inhibitory molecule.

213. The single chain peptide-major histocompatibility complex (pMHC) fusion molecule carrier of claim 212, wherein the T-cell inhibitory molecule is at least one ligand of BTLA, B7-1, B7-H1, CD160, CTLA4, LAG3, LAIR1, MHC-I, PD1, TIGIT, or TIM3.

214. The single chain peptide-major histocompatibility complex (pMHC) fusion molecule carrier of claim 211 or claim 213, wherein the ligand is a small molecule, protein, fusion protein, peptide, aptamer, avimer or derivatives or fragments thereof.

215. The single chain peptide-major histocompatibility complex (pMHC) fusion molecule carrier of any one of claims 204-214, wherein the pMHC complex comprises (i) a class I MHC polypeptide or a fragment, mutant or derivative thereof, and optionally, (ii) a β2 microglobulin polypeptide or a fragment, mutant or derivative thereof.

216. The single chain peptide-major histocompatibility complex (pMHC) fusion molecule carrier of claim 215, wherein the pMHC complex comprises a class I MHC polypeptide linked to a β2 microglobulin polypeptide via a peptide linker.

217. The single chain peptide-major histocompatibility complex (pMHC) fusion molecule carrier of any one of claims 204-216, wherein the pMHC complex comprises a human class I MHC polypeptide HLA-A, HLA-B, HLA-C, HLA-E, HLA-F, or HLA-G.

218. The single chain peptide-major histocompatibility complex (pMHC) fusion molecule carrier of any one of claims 204-216, wherein the pMHC complex comprises a murine class I MHC polypeptide H-2K, H-2D, H-2L, H-2Q, H-2M, or H-2T.

219. The single chain peptide-major histocompatibility complex (pMHC) fusion molecule carrier of any one of claims 204-216, wherein the single chain major histocompatibility complex (MHC)-peptide comprises (i) a peptide, (ii) a β2-microglobulin polypeptide or a fragment, mutant or derivative thereof, and (iii) a class I MHC alpha chain polypeptide or a fragment, mutant, or derivative thereof.

220. The single chain peptide-major histocompatibility complex (pMHC) fusion molecule carrier of any one of claims 204-216, wherein the single chain major histocompatibility complex (MHC)-peptide comprises (i) a peptide, (ii) a β2-microglobulin polypeptide or a fragment, mutant or derivative thereof, and (iii) a class I MHC alpha chain polypeptide or a fragment, mutant, or derivative thereof.

221. The single chain peptide-major histocompatibility complex (pMHC) fusion molecule carrier of claim 219, wherein the single chain major histocompatibility complex (MHC)-peptide comprises, from the N-terminus to the C-terminus, (i) a peptide, (ii) a β₂-microglobulin polypeptide or a fragment, mutant or derivative thereof, (iii) a class I MHC alpha chain or a fragment, mutant or derivative thereof, and (iv) a domain that specifically binds a molecule expressed on the surface of a carrier.

222. The single chain peptide-major histocompatibility complex (pMHC) fusion molecule carrier of claim 220, wherein the first molecule comprises, from N-terminus to C-terminus, (i) a peptide, (ii) an optional first linker, (iii) a β₂-microglobulin polypeptide or a fragment, mutant or derivative thereof, (iv) an optional linker, (v) class I MHC alpha chain domains 1, 2 and/or 3 or fragments, mutants or derivatives thereof, (vi) an optional third linker, and (vii) a domain specifically binds a molecule expressed on the surface of a carrier.

223. The single chain peptide-major histocompatibility complex (pMHC) fusion molecule carrier of any one of claims 204-214, wherein the pMHC complex comprises a class II MHC polypeptide or a fragment, mutant or derivative thereof.

224. The single chain peptide-major histocompatibility complex (pMHC) fusion molecule carrier of claim 223, wherein the pMHC complex comprises class II MHC alpha and beta chain polypeptides or a fragment, mutant or derivative thereof.

225. The single chain peptide-major histocompatibility complex (pMHC) fusion molecule carrier of claim 224, wherein the alpha and beta chain polypeptides are linked by a peptide linker.

226. The single chain peptide-major histocompatibility complex (pMHC) fusion molecule carrier of claim 224 or claim 225, wherein the alpha and beta chain polypeptides are from a human class II MHC HLA DP, HLA-DR, or HLA-DQ.

227. The single chain peptide-major histocompatibility complex (pMHC) fusion molecule carrier of claim 224 or claim 225, wherein the alpha and beta chain polypeptides are from a murine H-2A or H-2E class II MHC complex.

228. The single chain peptide-major histocompatibility complex (pMHC) fusion molecule carrier of any one of claims 204-227, wherein the domain that specifically binds a molecule expressed on the surface of the carrier comprises a Fab or single-chain Fv (scFv).

229. The single chain peptide-major histocompatibility complex (pMHC) fusion molecule carrier of claim 228, wherein the Fab or scFv specifically binds a molecule on the surface of the cell, virus, virus-like particle, or solid support.

230. The single chain peptide-major histocompatibility complex (pMHC) fusion molecule carrier of any one of claims 204-229, wherein the carrier is a cell.

231. The single chain peptide-major histocompatibility complex (pMHC) fusion molecule carrier of claim 230, wherein the cell is CHO, HEK 293, HMEC, HIVE-55, HIVS-125, or tumor cells.

232. The single chain peptide-major histocompatibility complex (pMHC) fusion molecule carrier of claim 230, wherein the cell is an antigen presenting cell.

233. The single chain peptide-major histocompatibility complex (pMHC) fusion molecule carrier of claim 232, wherein the cell is a B-cell.

234. The single chain peptide-major histocompatibility complex (pMHC) fusion molecule carrier of claim 233, wherein the B-cell surface molecule is at least one of CD5, CD19, CD20, CD21, CD22, CD23, CD24, CD25, CD27, CD30, CD34, CD35, CD38, CD180, and CD40.

235. The single chain peptide-major histocompatibility complex (pMHC) fusion molecule carrier of claim 234, wherein the B-cell surface molecule is CD20.

236. The single chain peptide-major histocompatibility complex (pMHC) fusion molecule carrier of any one of claims 204-229, wherein the carrier is a virus-like particle.

237. The single chain peptide-major histocompatibility complex (pMHC) fusion molecule carrier of any one of claims 204-229, wherein the carrier is a recombinant virus.

238. The single chain peptide-major histocompatibility complex (pMHC) fusion molecule carrier of claim 237, wherein the virus is derived from a retrovirus, an adenovirus, an adeno-associated virus, or a herpesvirus.

239. The single chain peptide-major histocompatibility complex (pMHC) fusion molecule carrier of claim 238, wherein the retrovirus is a lentivirus.

240. The single chain peptide-major histocompatibility complex (pMHC) fusion molecule carrier of any one of claim 204-215 or 223-239, wherein the single chain major histocompatibility complex (MHC)-peptide comprises (i) a peptide, (ii) a class II MHC alpha chain polypeptide or a fragment, mutant or derivative thereof, and (iii) a class II MHC beta chain polypeptide or a fragment, mutant or derivative thereof, and (iv) at least one domain that specifically binds a molecule expressed on the surface of a carrier.

241. The single chain peptide-major histocompatibility complex (pMHC) fusion molecule carrier of claim 240, wherein the class II MHC alpha chain and class II MHC beta chain share one domain that specifically binds a molecule expressed on the surface of a carrier.

242. The single chain peptide-major histocompatibility complex (pMHC) fusion molecule carrier of claim 240, wherein the class II MHC alpha chain and class II MHC beta chain each have a separate domain that specifically binds a molecule expressed on the surface of a carrier.

243. The single chain peptide-major histocompatibility complex (pMHC) fusion molecule carrier of claim 242, wherein the separate domains that specifically binds a molecule expressed on the surface of a carrier are the same.

244. The single chain peptide-major histocompatibility complex (pMHC) fusion molecule carrier of claim 242, wherein the separate domains that specifically binds a molecule expressed on the surface of a carrier are different.

245. The single chain peptide-major histocompatibility complex (pMHC) fusion molecule carrier of any one of claim 204-215 or 223-239, wherein the pMHC complex comprises MHC class II alpha chain extracellular domain or a fragment, mutant or derivative thereof and a domain that specifically binds a molecule expressed on the surface of a carrier.

246. The single chain peptide-major histocompatibility complex (pMHC) fusion molecule carrier of any one of claim 204-215 or 223-239, wherein the pMHC complex comprises MHC class II beta chain extracellular domain or a fragment, mutant or derivative thereof and a domain that specifically binds a molecule expressed on the surface of a carrier.

247. The single chain peptide-major histocompatibility complex (pMHC) fusion molecule carrier of claim 245 or claim 246, wherein the first molecule comprises, from the N-terminus to the C-terminus, (i) a peptide, (ii) class II MHC alpha chain extracellular domains or fragments, mutants or derivatives thereof, (iii) class II MHC beta chain extracellular domains or fragments, mutants or derivatives thereof, and (iv) a domain that specifically binds a molecule expressed on the surface of a carrier.

248. The single chain peptide-major histocompatibility complex (pMHC) fusion molecule carrier of claim 245 or claim 246, wherein the first molecule comprises, from the N-terminus to the C-terminus, (i) a peptide, (ii) class II MHC beta chain extracellular domains or fragments, mutants or derivatives thereof, (iii) class II MHC alpha chain extracellular domains or fragments, mutants or derivatives thereof, and (iv) a domain that specifically binds a molecule expressed on the surface of a carrier

249. The single chain peptide-major histocompatibility complex (pMHC) fusion molecule carrier of claim 247, wherein the first molecule comprises, from the N-terminus to the C-terminus, (i) a peptide, (ii) an optional first linker, (iii) class II MHC alpha chain domains 1 and 2 or fragments, mutants or derivatives thereof, (iv) an optional second linker, (v) class II MHC beta chain domains 1 and 2 or fragments, mutants or derivatives thereof, (vi) an optional third linker, and (vii) a domain that specifically binds a molecule expressed on the surface of a carrier.

250. The single chain peptide-major histocompatibility complex (pMHC) fusion molecule carrier of claim 248, wherein the first molecule comprises, from the N-terminus to the C-terminus, (i) a peptide, (ii) an optional first linker, (iii) class II MHC beta chain domains 1 and 2 or fragments, mutants or derivatives thereof, (iv) an optional second linker, (v) class II MHC alpha chain domains 1 and 2 or fragments, mutants or derivatives thereof, (vi) an optional third linker, and (vii) a domain that specifically binds a molecule expressed on the surface of a carrier.

251. The single chain peptide-major histocompatibility complex (pMHC) fusion molecule carrier of any one of claims 204-250, wherein the peptide consists of from about 5 to about 40 amino acid residues.

252. The single chain peptide-major histocompatibility complex (pMHC) fusion molecule carrier of claim 251, wherein the peptide consists of from about 6 to about 30 amino acid residues.

253. The single chain peptide-major histocompatibility complex (pMHC) fusion molecule carrier of claim 252, wherein the peptide consists of from about 8 to about 20 amino acid residues.

254. The single chain peptide-major histocompatibility complex (pMHC) fusion molecule carrier of claim 253, wherein the peptide consists of from about 9 to about 11 amino acid residues.

255. The single chain peptide-major histocompatibility complex (pMHC) fusion molecule carrier of any one of claims 204-254, wherein the peptide is derived from a viral antigen or a bacterial antigen.

256. The single chain peptide-major histocompatibility complex (pMHC) fusion molecule carrier of claim 255, wherein he viral antigen is derived from a virus, wherein the virus is adenovirus, astrovirus, chikungunya, cytomegalo, dengue, ebola, EBV, hantavirus, HBsAg, hepatitis A, hepatitis B, hepatitis C, hepatitis D, hepatitis E, herpes, HIV, HPIV, HTLV, influenza, Japanese encephalitis virus, lassa, measles, metapneumovirus, mumps, norovirus, oropauche, HPV, parvovirus, rotavirus, RSV, rubella, SARS, TBEV, usutu, vaccina, varicella, West Nile, yellow fever, or zika.

257. The single chain peptide-major histocompatibility complex (pMHC) fusion molecule carrier of claim 255, wherein the bacterial antigen is derived from a bacterium, wherein the bacterium is methicillin-resistant Staphylococcus Aureus (MRSA), Clostridium Difficile, carbapenum-resistant Enterobacteriaceae, drug-resistant Neisseria Gonorrhoeae, multidrug-resistant Acinetobacter, drug-resistant Campylobacter, Fluconazole-resistant Candida, extended-spectrum B-lactamase producing bacteria, Vancomycin-resistant enterococcus, multidrug-resistant pseudomonas Aeruginosa, drug-resistant non-typhoidal Salmonella, drug-resistant Salmonella serotype typhi, drug-resistant Shigella, drug-resistant Streptococcus Pneumoniae, drug-resistant tuberculosis, Vancomycin-resistant Staphylococcus Aureus, Erythomycin-resistant group A Streptococcus, or Clindamycin-resistant group B Streptococcus.

258. The single chain peptide-major histocompatibility complex (pMHC) fusion molecule carrier of any one of claims 204-254, wherein the peptide is derived from a tumor-associated antigen.

259. The single chain peptide-major histocompatibility complex (pMHC) fusion molecule carrier of claim 258, wherein the tumor-associated antigen is adipophilin, AIM-2, ALDH1A1, alpha-actinin-4, alpha-fetoprotein (“AFP”), ARTC1, ALK, BAGE proteins (e.g., BAGE-1), BIRC5 (survivin), BIRC7, β-catenin, BRCA1, BORIS, B-RAF, BCLX (L), BCR-ABL fusion protein b3a2, beta-catenin, BING-4, CA-125, CALCA, carcinoembryonic antigen (“CEA”), CAGE-1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, CA9, carbonic anhydrase IX, caspase-8, CALR, CCR5, CD19, CD20 (MS4A1), CD22, CD40, CD70, CDK4, cyclin-B1, CYP1B1, dek-can fusion protein, DKK1, EFTUD2, Elongation factor 2, ENAH (hMena), EphA3, epithelial tumor antigen (“ETA”), EGFR, EGFRVIII, ErbB2/Her2, ErbB3, ErbB4, ETV6-AML1 fusion protein, EpCAM, EphA2, EZH2, FGF5, FLT3-ITD, FN1, Fra-1, FOLR1, G250/MN/CAIX, GAGE proteins (e.g., GAGE-1-8), GD2, GD3, GloboH, glypican-3, GM3, gp100, GAS7, GnTV, gp100/Pme117, GPNMB, GnTV, HAUS3, Hepsin, HERV-K-MEL, HLA-A11, HLA-A2, HLA-DOB, hsp70-2, HPV E2, HPV E6, HPV E7, HPV EG, Her2/neu, HLA/B-raf, HLA/k-ras, HLA/MAGE-A3, hTERT, IDO1, IGF2B3, IL13Ralpha2, Intestinal carboxyl esterase, K-ras, Kallikrein 4, KIF20A, KK-LC-1, KKLC1, KM-HN-1, KMHN1 also known CCDC110, LAGE-1, LDLR-fucosyltransferaseAS fusion protein, Lengsin, LMP2, M-CSF, MAGE proteins (e.g., MAGE-A1,-A2,-A3,-A4,-A6,-A9,-A10,-A12,-C1, and C2), malic enzyme, mammaglobin-A, MART-1, MART-2, MATN, MC1R, MCSP, mdm-2, ME1, Melan-A/MART-1, Meloe, Midkine, MMP-2, MMP-7, mesothelin, ML-IAP, Muc1, Muc2, Muc3, Muc4, Muc5, Muc16 (CA-125), MUC5AC, MUM-1, MUM-2, MUM-3, Myosin, Myosin class I, N-raw, NA88-A, neo-PAP, NFYC, NA17, NA-88, NY-BR1, NY-BR62, NY-BR85, NY-ESO1/LAGE-2, OA1, OGT, OS-9, P polypeptide, p15, p53, PAP, PAX3, PAX5, PCTA-1, PLAC1, PRLR, PRAME, PSMA (FOLH1), PBF, pml-RARalpha fusion protein, polymorphic epithelial mucin (“PEM”), PPP1R3B, PRDX5, PSA, PSMA, PTPRK, RAB38/NY-MEL-1, RBAF600, RGS5, RhoC, RNF43, RU2AS, RAGE proteins (e.g., RAGE-1), Ras, RGS5, Rho, SART-1, SART-3, STEAP1, STEAP2, SAGE, secernin 1, SIRT2, SNRPD1, SOX10, Sp17, SPA17, SSX-2, SSX-4, STEAP1, survivin, SYT-SSX1 or SSX2 fusion protein, TAG-1, TAG-2, TAG-72, TGF-β, TMPRSS2, Thompson-nouvelle antigen (Tn), TRP-1/gp75, TRP-2, TRP2-INT2, tyrosinase, Telomerase, TPBG, TRAG-3, Triosephosphate isomerase, uroplakin-3, VEGF, XAGE-1b/GAGED2a, or WT-1.

260. The single chain peptide-major histocompatibility complex (pMHC) fusion molecule carrier of any one of claims 204-254, wherein the peptide is derived from an antigen associated with an autoimmune disorder.

261. The single chain peptide-major histocompatibility complex (pMHC) fusion molecule carrier of claim 260, wherein the antigen associated with an autoimmune disorder is gliadin, GAD 65, IA-2, insulin β chain, glatiramer acetate (GA), acetylcholine receptor (AChR), p205, insulin, thyroid-stimulating hormone, tyrosinase, TRP1, or myelin antigens.

262. The single chain peptide-major histocompatibility complex (pMHC) fusion molecule carrier of claim 260, wherein the antigen associated with an autoimmune disorder is IL-4R, IL-6R, or DLL4.

263. A pharmaceutical composition comprising the single chain major histocompatibility complex (MHC)-peptide carrier of any one of claims 204-262, and a pharmaceutically acceptable carrier or diluent.

264. A method of engineering a population of cells to express transmembrane polypeptides to modulate an activity an activity or survival of a cell expressing a T cell receptor (TCR), comprising:
(a) providing a population of cells;
(b) introducing into the cells one or more nucleic acid molecules encoding the multispecific molecule of any one of claims 1-61 or the single chain major histocompatibility complex (MHC)-peptide of any one of claim 153-202 or 204-262;
(c) culturing the cells under conditions to express said one or more nucleic acid molecules; and
(d) isolating the cells expressing the multispecific molecule.

265. The method of claim 264, wherein the cells are B cells.

266. A nucleic acid molecule encoding the multispecific molecule of any one of claims 1-61 or the single chain major histocompatibility complex (MHC)-peptide of any one of claim 153-202 or 204-262.

267. A nucleic acid molecule encoding the first molecule of the multispecific molecule of any one of claims 1-61.

268. A nucleic acid molecule encoding the second molecule of the multispecific molecule of any one of claims 1-61.

269. A nucleic acid molecule encoding the single chain peptide-major histocompatibility complex (pMHC) fusion molecule of any one of claim 153-202 or 204-262.

270. A vector comprising the nucleic acid molecule according to any one of claims 266-269.

271. The vector of claim 270, wherein the vector is a DNA vector or an RNA vector.

272. The vector of claim 271, wherein the vector is a plasmid.

273. The vector of claim 271, wherein the vector is a retroviral vector.

274. The vector of claim 271, wherein the retroviral vector is a lentiviral vector.

275. A method of engineering a population of cells to express at least two separate transmembrane polypeptides to modulate an activity an activity or survival of a cell expressing a T cell receptor (TCR), comprising:
(a) providing a population of cells;
(b) introducing into the cells one or more nucleic acid molecules encoding a first molecule comprising a peptide (p) presented in the context of a major histocompatibility complex (MHC) molecule (pMHC complex);
(c) introducing into the cells one or more nucleic acid molecules encoding a second molecule comprising a domain that specifically binds a molecule expressed on the surface of the cell expressing the TCR;
(d) culturing the cells under conditions to express said one or more first set and one or more second set of nucleic acid molecules; and
(e) isolating the cells expressing the first and second molecules.

276. The method of claim 275, wherein the cells are B cells.

277. A nucleic acid molecule encoding the multispecific molecule encoding a first molecule comprising a peptide (p) presented in the context of a major histocompatibility complex (MHC) molecule (pMHC complex).

278. A nucleic acid molecule encoding the multispecific molecule encoding a second molecule comprising a domain that specifically binds a molecule expressed on the surface of the cell expressing the TCR.

279. A nucleic acid molecule encoding the first molecule expressed on the surface of the multispecific carrier of any one of claims 66-136.

280. A nucleic acid molecule encoding the second molecule expressed on the surface of the multispecific carrier of any one of claims 66-136.

281. A vector comprising the nucleic acid molecule according to any one of claims 277-280.

282. The vector of claim 281, wherein the vector is a DNA vector or an RNA vector.

283. The vector of claim 281, wherein the vector is a plasmid.

284. The vector of claim 281, wherein the vector is a retroviral vector.

285. The vector of claim 281, wherein the retroviral vector is a lentiviral vector.

286. The multimeric molecule of claim 61 comprising a clustering domain, wherein said clustering domain comprises a binding region or linker molecule to enable the multispecific molecules to be physically conjoined.

287. The multimeric molecule of claim 286, wherein the clustering domain comprises an IgG1 Fc domain or IgG4 Fc.

288. The multimeric molecule of claim 286, wherein the clustering domain comprises a scFv that binds to a cell surface molecule.

289. The multimeric molecule of claim 286, wherein the clustering domain comprises an amino acid linker.

290. The multimeric molecule of claim 289, wherein the amino acid linker binds the multispecific molecules to (i) an immunoglobulin multimer or (ii) a cell surface or (iii) a virus-like particle or (iv) a solid support.

291. A method of modulating an activity, proliferation or survival of a cell expressing a T cell receptor (TCR), comprising contacting the cell with an effective amount of one or more of (i) the multispecific molecule of any one of claims 1-60, (ii) the multimeric multispecific molecule of any one of claims 61 and 286-290, (iii) the single chain peptide-major histocompatibility complex (pMHC) fusion molecule of any one of claims 153-202, (iv) the liposome of claim 63 or claim 64, (v) the multispecific carrier of any one of claims 66-136, the single chain peptide-major histocompatibility complex (pMHC) fusion molecule carrier of any one of claims 204-262, (vi) the virus-like particle of claim 138 or claim 139, (vii) the recombinant virus of any one of claims 141-144, (viii) the solid support of any one of claims 146-148, (ix) the engineered cell of any one of claims 149-151, or (x) the pharmaceutical composition of any one of claim 65, 137, 140, 145, 152, 203, or 263.

292. The method of claim 291, wherein the TCR is antigen specific, and wherein the multispecific molecule or first molecule of the multispecific carrier binds the idiotype of the TCR antigen recognition site.

293. The method of claim 291 or claim 292, wherein the cell is a lymphocyte.

294. The method of claim 293, wherein the lymphocyte is a T-cell.

295. The method of claim 294, wherein the T-cell is a CD8⁺ T-cell.

296. The method of claim 295, wherein the contacting results in modulation of activation, proliferation and/or survival of the CD8⁺ T-cell.

297. The method of any one of claims 291-296, wherein the multispecific molecule or first molecule of the multispecific carrier binds to a T-cell co-stimulatory molecule.

298. The method of claim 297, wherein the T-cell co-stimulatory molecule is CD2, CD27, CD28, CD30, CD40L, CD226, DR3, galectin 9, GITR, HVEM, ICOS, LFA1, OX40, SLAM, TIM1, TIM2, 2B4, or 4-1BB.

299. The method of any one of claims 291-296, wherein the multispecific molecule binds to a T-cell inhibitory molecule, and whereby the contacting inhibits CD8⁺ T-cell activation, proliferation and/or survival.

300. The method of claim 299, wherein the inhibitory molecule is at least one of BTLA, B7-1 and B7-H1, CD160, CTLA4, LAG3, LAIR1, MHC-I, PD1, TIGIT, or TIM3.

301. The method of any one of claims 291-300, wherein the inhibition of CD8⁺ T-cell activation, proliferation and/or survival results in induction of T cell anergy or T cell death.

302. The method of claim 301, wherein the T-cell is a CD4+ T-cell.

303. The method of claim 302, wherein the contacting results in modulation of activation, proliferation and/or survival of the CD4⁺ T-cell.

304. The method of any one of claims 291-303, wherein said contacting is conducted ex vivo.

305. The method of claim 304, wherein the cell expressing a T cell receptor (TCR) has been isolated from a subject.

306. The method of claim 304, wherein the contacting occurs under conditions and for a period of time sufficient to modulate activity, proliferation or survival of the cell expressing a T cell receptor (TCR) and is followed by introducing the modulated cell back into the same subject or into a different subject.

307. The method of any one of claims 291-303, wherein said contacting is conducted in vivo in a subject.

308. The method of any one of claims 291-307, wherein the subject is human.

309. The method of any one of claims 291-307, wherein the cell is a mammalian cell.

310. The method of claim 309, wherein the mammalian cell is a human cell.

311. A method of treating a disorder in a subject in need thereof comprising administering to said subject an effective amount of one or more of (i) the multispecific molecule of any one of claims 1-60, (ii) the multimeric multispecific molecule of any one of claims 61 and 286-290, (iii) the single chain peptide-major histocompatibility complex (pMHC) fusion molecule of any one of claims 153-202, (iv) the liposome of claim 63 or claim 64, (v) the multispecific carrier of any one of claims 66-136, the single chain peptide-major histocompatibility complex (pMHC) fusion molecule carrier of any one of claims 204-262, (vi) the virus-like particle of claim 138 or claim 139, (vii) the recombinant virus of any one of claims 141-144, (viii) the solid support of any one of claims 146-148, (ix) the engineered cell of any one of claims 149-151, or (x) the pharmaceutical composition of any one of claim 65, 137, 140, 145, 152, 203, or 263.

312. The method of claim 311, wherein the disorder is an infection caused by an infectious agent and wherein the administration results in enhancement of an immune response against the infectious agent.

313. The method of claim 312, wherein the infectious agent is at least one of a virus, a bacterium, a fungus, a protozoan, a parasite, a helminth, and an ectoparasite.

314. The method of claim 312, wherein the agent causing the infection is a virus, and the peptide is a fragment of a viral protein.

315. The method of claim 312, wherein the infectious agent is lymphocytic choriomeningitis virus (LCMV) and the antigen is glycoprotein (gp33) or a fragment thereof.

316. The method of any one of claims 311-315, wherein the multispecific molecule or second molecule of the multispecific carrier specifically binds to a T-cell co-stimulatory molecule.

317. The method of any one of claims 311-316, wherein the multispecific molecule or second molecule of the multispecific carrier specifically binds to CD2, CD27, CD28, CD30, CD40L, CD226, DR3, galectin 9, GITR, HVEM, ICOS, LFA1, OX40, SLAM, TIM1, TIM2, 2B4, or 4-1BB.

318. The method of claim 311, wherein the disorder is cancer and wherein the administration results in enhancement of an anti-tumor immune response.

319. The method of claim 318, wherein the multispecific molecule or second molecule of the multispecific carrier specifically binds to a T-cell co-stimulatory molecule.

320. The method of claim 319, wherein the multispecific molecule or second molecule of the multispecific carrier specifically binds to CD2, CD27, CD28, CD30, CD40L, CD226, DR3, galectin 9, GITR, HVEM, ICOS, LFA1, OX40, SLAM, TIM1, TIM2, 2B4, or 4-1BB.

321. The method of claim 311, wherein the disorder is an inflammatory or an autoimmune disorder and wherein the administration results in inhibition of an inflammatory or an autoimmune response.

322. The method of claim 321, wherein the disorder is celiac disease or gluten sensitivity.

323. The method of claim 322, wherein the antigen comprises a gliadin or a fragment thereof.

324. The method of any one of claims 321-323, wherein the multispecific molecule or second molecule of the multispecific carrier specifically binds to a T-cell inhibitory molecule.

325. The method of any one of claims 321-324, wherein the multispecific molecule or second molecule of the multispecific carrier specifically binds to BTLA, B7-1 and B7-H1, CD160, CTLA4, LAG3, LAIR1, MHC-I, PD1, TIGIT, or TIM3.

326. The method of any one of claims 311-325, wherein the subject is a mammal.

327. The method of claim 326, wherein the mammal is human.

328. The method of any one of claims 291-327, wherein the plurality of multispecific molecules are bound to a scaffold in a clustered arrangement.

329. The method of any one of claims 291-327, wherein the plurality of multispecific molecules are clustered via one or more linkers.

330. The method of claim 329, wherein the one or more linkers is a multivalent antibody that binds the first and/or the second Fc domain of the multispecific molecules.

331. The method of claim 330, wherein the ratio of multivalent antibodies to multispecific molecules is about 1:1 in the culture.
